(19) United States
(12) Patent Application Publication
(10) Pub. No.: US 2022/0275341 A1
(43) Pub. Date: Sep. 1, 2022

(54) ORGANOID MESODERM LINEAGE DIVERSIFICATION

(71) Applicants: CHILDREN'S HOSPITAL MEDICAL CENTER, Cincinnati, OH (US); RIKEN, Wako, Saitama (JP)

(72) Inventors: Aaron M. Zorn, Cincinnati, OH (US); Lu Han, Charleston, SC (US); Keishi Kishimoto, Wako, Saitama (JP); Mitsuru Morimoto, Wako, Saitama (JP)

(21) Appl. No.: 17/638,753

(22) PCT Filed: Aug. 25, 2020
(86) PCT No.: PCT/US2020/047846

§ 371 Date: Feb. 25, 2022

Related U.S. Application Data
(60) Provisional application No. 62/892,781, filed on Aug. 28, 2019.

(51) Int. Cl. C12N 5/071; C12N 5/077

(52) U.S. Cl. CPC C12N 5/0679; C12N 5/0652; C12N 2501/15; C12N 2501/415; C12N 2501/155; C12N 2501/115; C12N 2501/999; C12N 2506/02

(57) ABSTRACT

Disclosed herein are in vitro methods of making splanchnic mesoderm cell types and subtypes thereof from pluripotent cells. These methods can be used to produced improved foregut- and hindgut-derived organoids containing enriched mesenchyme, which enhances organoid viability, growth, and maturation, both in in vitro culture and in vivo transplantation.

### CROSS-REFERENCE TO RELATED APPLICATIONS

This application claims the benefit of priority of U.S. Provisional Patent Application No. 62/892,781, filed Aug. 28, 2019, which is hereby expressly incorporated by reference in its entirety.

### STATEMENT REGARDING FEDERALLY SPONSORED R&D

This invention was made with government support under P01HD093363 and P30 DK078392 awarded by the National Institutes of Health. The government has certain rights to the invention.

### FIELD OF THE INVENTION

Aspects of the present disclosure relate generally to new and improved methods of differentiating splanchnic mesoderm and subtypes thereof from pluripotent stem cells.

### BACKGROUND

In early fetal development, between embryonic day (E) 8.5 and E9.5 in mouse, equivalent to 17-23 days of human gestation, a series of inductive tissue interactions between the definitive endoderm (DE) and the surrounding splanchnic mesoderm (SM) progressively patterns the naive foregut tube into different progenitor domains. These domains further develop into distinct visceral organs including the trachea., lung, esophagus, stomach, liver, pancreas and proximal small intestine. The DE gives rise to the epithelial lining and parenchyma of the respiratory and digestive organs, while the SM gives rise to the mesenchymal tissues such as smooth muscle, fibroblasts and mesentery surrounding the visceral organs. This foregut patterning defines the landscape of the thoracic and abdominal cavities, setting the relative position of different organs. Disruptions in this process can lead to life threatening congenital birth defects. There is a present need for a greater understanding of mesoderm differentiation during embryogenesis and improved methods of differentiating mesoderm in vitro using pluripotent stem cells (PSCs), such as patient-derived PSCs, with applications towards, for example, the production of improved organoids for genetics, drug screening, personalized medicine, and transplantation.

### SUMMARY

Disclosed herein are methods of producing splanchnic mesoderm cells. The methods comprise contacting lateral plate mesoderm cells with a TGF-beta signaling pathway inhibitor, a Wnt signaling pathway inhibitor, a BMP signaling pathway activator, an FGF signaling pathway activator, and a retinoic acid (RA) signaling pathway activator, thereby differentiating the lateral plate mesoderm cells to splanchnic mesoderm cells. In some embodiments, the splanchnic mesoderm cells are human splanchnic mesoderm cells. In some embodiments, the lateral plate mesoderm cells have been differentiated from middle primitive stream cells. In some embodiments, the lateral plate mesoderm cells have been differentiated from middle primitive streak cells by contacting the middle primitive streak cells with a TGF-beta signaling pathway inhibitor, a Wnt signaling pathway inhibitor, and a BMP signaling pathway activator. In some embodiments, the middle primitive streak cells have been differentiated from pluripotent stem cells. In some embodiments, the middle primitive streak cells have been differentiated from pluripotent stem cells by contacting the pluripotent stem cells with a TGF-beta signaling pathway activator, a Wnt signaling pathway activator, an FGF signaling pathway activator, a BMP signaling pathway activator, and a PI3K signaling pathway inhibitor. In some embodiments, the lateral plate mesoderm cells are contacted with A8301, BMP4, C59, FGF2, RA, or any combination thereof. In some embodiments, the lateral plate mesoderm cells are contacted for a time that is sufficient to differentiate lateral plate mesoderm cells to splanchnic mesoderm cells, and/or for a time that is or is about 36, 37, 38, 39, 40, 41, 42, 43, 44. 45, 46, 47. 48, 49, 50, 51, 52, 53, 54, 55, 56, 57, 58, 59, or 60 hours, or any time within a range defined by any two of the aforementioned times. In some embodiments, the lateral plate mesoderm cells are contacted for a time that is or is about 48 hours. In some embodiments, the splanchnic mesoderm cells exhibit increased expression of FOXF1, HOXA1, HOXA5, or WNT2, or any combination thereof, and decreased expression of NKX2-5, NH or TBX2, or any combination thereof, relative to cardiac mesoderm cells. In some embodiments, the splanchnic mesoderm cells exhibit decreased expression of PAX3 or PRRX1, or both, relative to middle primitive streak cells, and/or decreased expression of CD31 relative to cardiac mesoderm cells.

Also disclosed herein are methods of producing septum transversum cells. The methods comprise contacting splanchnic mesoderm cells with a retinoic acid signaling pathway activator and a BMP signaling pathway activator. In some embodiments, the splanchnic mesoderm cells are any of the splanchnic mesoderm cells disclosed herein. In some embodiments, the splanchnic mesoderm cells are contacted with RA, BMP4, or both. In some embodiments, the splanchnic mesoderm cells are contacted for a period of time that is or is about 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times. In some embodiments, the splanchnic mesoderm cells are contacted for a period of time that is or is about 72 hours. In some embodiments, the septum transversum cells exhibit increased expression of WT1, TBX18, LHX2, UPK3B, or UPK1B, or any combination thereof, relative to cardiac mesoderm cells, splanchnic mesoderm cells, or fibroblasts, or any combination thereof. In some embodiments, the septum transversum cells exhibit decreased expression of MSX1, MSX2, or HAND1, or any combination thereof, relative to cardiac mesoderm cells or fibroblasts, or both. In some embodiments, the septum transversum cells exhibit decreased expression of HOXA1 or TBX5, or both, relative to splanchnic mesoderm cells. In some embodiments, the septum transversum cells exhibit decreased expression of NKX6.1 or HOXA5, or both, relative to respiratory mesenchyme cells. In some embodiments, the septum transversum cells exhibit decreased expression of NKX3.2, MSC, BARX1, WNT4, or HOXA5, or any combination thereof, relative to esophageal/gastric mesenchyme cells. In some embodiments, the septum transversum cells account for about 60%, 65%, 70%, 75%, 80%, 85%, or 90% of the total cells differentiated from the splanchnic mesoderm cells.

Also disclosed herein are methods of producing fibroblasts. The methods comprise contacting splanchnic mesoderm cells with a retinoic acid signaling pathway activator, a BMP signaling pathway activator, and a Wnt signaling pathway activator. In some embodiments, the splanchnic mesoderm cells are any of the splanchnic mesoderm cells disclosed herein. In some embodiments, the splanchnic mesoderm cells are contacted with RA, BMP4, C1THR99021, or any combination thereof. In some embodiments, the fibroblasts are liver fibroblasts. In some embodiments, the splanchnic mesoderm cells are contacted for a period of time that is or is about 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times. In some embodiments, the splanchnic mesoderm cells are contacted for a period of time that is or is about 72 hours. In some embodiments, the fibroblasts exhibit increased expression of MSX1, MSX2, or HAND1, or any combination thereof, relative to splanchnic mesoderm cells or septum transversum cells, or both. In some embodiments, the fibroblasts exhibit decreased expression of WT1, TBX18, LHX2, or UPK1B, or any combination thereof, relative to septum transversum cells. In some embodiments, the fibroblasts exhibit decreased expression of NKX6.1, HOXA5, or LHX2, or any combination thereof, relative to respiratory mesenchyme cells. In some embodiments, the fibroblasts exhibit decreased expression of NKX3.2 MSC, BARX1, WNT4, or HOXA5, or any combination thereof, relative to esophageal/gastric mesenchyme cells.

Also disclosed herein are methods of producing respiratory mesenchyme cells. The methods comprise a) contacting splanchnic mesoderm cells with a retinoic acid signaling pathway activator, a BMP signaling pathway activator, a hedgehog (HH) signaling pathway activator, and a Wnt signaling pathway activator. In some embodiments, the splanchnic mesoderm cells are contacted for a period of time that is or is about 60, 61, 62, 63, 64, 65, 66, 67, 68, 69, 70, 71, 72. 73, 74, 75, 76, 77, 78, 79, 80, 81, 82, 83, or 84 hours, or any period of time within a range defined by any two of the aforementioned times. In some embodiments, the splanchnic mesoderm cells are contacted for a period of time that is or is about 72 hours. In some embodiments, step a) is a second step, and further comprising a first step of contacting the splanchnic mesoderm cells with a retinoic acid signaling pathway activator, a BMP signaling pathway activator, and a signaling pathway activator prior to the second step. In some embodiments, the splanchnic mesoderm cells are contacted for a period of time that is or is about 36, 37, 38, 39, 40, 41, 42, 43, 44, 45, 46, 47, 48, 49, 50, 51, 52, 53, 54, 55, 56, 57, 58, 59, or 60 hours, or any period of time within a range defined by any two of the aforementioned times for the first step. In some embodiments, the splanchnic mesoderm cells are contacted for a period of time that is or is about 48 hours for the first step. In some embodiments, the splanchnic mesoderm cells are contacted for a period of time that is or is about 12, 13, 14, 15, 16, 17, 18, 19, 20, 21, 22, 23, 24, 25, 26, 27, 28, 29, 30, 31, 32, 33, 34, 35, or 36 hours, or any period of time within a range defined by any two of the aforementioned times for the second step. In some embodiments, the splanchnic mesoderm cells are contacted for a period of time that is or is about 24 hours for the second step. In some embodiments, the splanchnic mesoderm cells are any of the splanchnic mesoderm cells disclosed herein. In some embodiments, the splanchnic mesoderm cells are contacted with RA, BMP4, PMA, CHIR99021, or any combination thereof. In some embodiments, the respiratory mesenchyme cells exhibit increased expression of NKX6-1, TBX5, HOXA1, HOXA5, FOXF1, LHX2, or WNT2, or any combination thereof, relative to cardiac endoderm cells, splanchnic mesoderm cells, or esophageal,/gastric mesenchyme cells, or any combination thereof in some embodiments, the respiratory mesenchyme cells exhibit decreased expression of WNT2, WT1, TBX18, LHX2, or UPK1B, or any combination thereof, relative to septum transversum cells. In some embodiments, the respiratory mesenchyme cells exhibit decreased expression of WNT2, MSX1, or MSX2, or any combination thereof, relative to fibroblast cells.

Also disclosed herein are methods of producing esophageal/gastric mesenchyme cells. The methods comprise a) contacting splanchnic mesoderm cells with a retinoic acid signaling pathway activator, a BMP signaling pathway inhibitor, and a HH signaling pathway activator. In some embodiments, the splanchnic mesoderm cells are contacted for a period of time that is or is about 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times. In some embodiments, the splanchnic mesoderm cells are contacted for a period of time that is or is about 72 hours. In sonic embodiments, step a) is a second step, and further comprising a first step of contacting the splanchnic mesoderm cells with a retinoic acid signaling pathway activator and a HH signaling pathway activator prior to the second step. In some embodiments, the splanchnic mesoderm cells are contacted for a period of time that is or is about 36, 37, 38, 39, 40, 41, 42, 43, 44, 45, 46, 47, 48, 49, 50, 51, 52, 53, 54, 55, 56, 57, 58, 59, or 60 hours, or any period of time within a range defined by any two of the aforementioned times for the first step. In sonic embodiments, the splanchnic mesoderm cells are contacted for a period of time that is or is about 48 hours for the first step. In some embodiments, the splanchnic mesoderm cells are contacted fora period of time that is or is about 12, 13, 14, 15, 16, 17, 18, 19, 20, 21, 22, 23, 24, 25, 26, 27, 28, 29, 30, 31, 32, 33, 34, 35, or 36 hours, or any period of time within a range defined by any two of the aforementioned times for the second step. In some embodiments, the splanchnic mesoderm cells are contacted for a period of time that is or is about 24 hours for the second step. In some embodiments, the splanchnic mesoderm cells are any of the splanchnic mesoderm cells disclosed herein. In some embodiments, the splanchnic mesoderm cells are contacted with RA, Noggin, PMA, or any combination thereof. In some embodiments, the esophageal/gastric mesenchyme cells exhibit increased expression of MSC, BARX1, WNT4, HOXA1, FOXF1, or NKX3-2, or any combination thereof, relative to cardiac endoderm cells, splanchnic mesoderm cells, or respiratory mesenchyme cells, or any combination thereof. In some embodiments, the esophageal,/gastric mesenchyme cells exhibit decreased expression of WNT2, TBX5, MSX1, MSX2, or LHX2, or any combination thereof, relative to septum transversum cells, fibroblasts, or respiratory mesenchyme cells, or any combination thereof.

In any of the embodiments, the TGF-beta signaling pathway inhibitor is selected from the group consisting of A8301, RepSox, LY365947, and SB431542. In any of the embodiments, the TGF-beta signaling pathway inhibitor is A8301. In any of the embodiments, the TGF-beta signaling pathway inhibitor is contacted at a concentration of 0.1, 0.2, 0.3, 0.4, 0,5, 0.6, 0,7, 0.8, 0.9, 1, 1.1, 1.2, 1.3, 1.4, 1.5, 1.6, 1.7, 1.8, 1.9, or 2 μM, or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations. In any of the embodiments, the TGF-beta signaling pathway inhibitor is contacted at concentration of 1 μM or about 1 μM.

In any of the embodiments, the Wnt signaling pathway inhibitor is selected from the group consisting of C59, PNU 74654, KY-02111, PRI-724, FH-535 DIF-1, and XAV939. In any of the embodiments, the Wnt signaling pathway inhibitor is C59. In any of the embodiments, the Wnt signaling pathway inhibitor is contacted at a concentration of 0.1, 0.2, 0.3, 0.4, 0.5, 0.6, 0.7. 0.8, 0.9, 1. 1.1, 1.2, 1.3, 1.4, 1.5, 1.6, 1.7, 1.8, 1.9, or 2 μM, or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations. In any of the embodiments, the Wnt signaling pathway inhibitor is contacted at a concentration of 1 μM or about 1 μM.

In any of the embodiments, the BMP signaling pathway activator is selected from the group consisting of BMP1, BMP2, BMP3, BMP4, BMP5, BMP6, BMP7, BMP8a, BMP8b, BMP10, BMP11, BMP15, IDE1, and IDE2. In any of the embodiments, the BMP signaling pathway activator is BMP4. In any of the embodiments, the BMP signaling pathway activator is contacted at a concentration of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/mL or about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/mL, or any concentration within a range defined by any two of the aforementioned concentrations. In any of the embodiments, the BMP signaling pathway activator is contacted at a concentration of 30 ng/mL or about 30 ng/mL.

In any of the embodiments, the FGF signaling pathway activator is selected from the group consisting of FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, and FGF23. In any of the embodiments, the FGF signaling pathway activator is FGF2. In any of the embodiments, the FGF signaling pathway activator is contacted at a concentration of 5, 6, 7, 8, 9, 10, 11, 12, 13, 14, 15, 16, 17, 18, 19, 20, 21, 22, 23, 24, 25, 26, 27, 28, 29. 30, 31, 32, 33, 34, or 35 ng/mL, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 ng/mL, or any concentration within a range defined by any two of the aforementioned concentrations. In any of the embodiments, the FGF signaling pathway activator is contacted at a concentration of 20 ng/mL or about 20 ng/mL.

In any of the embodiments, the RA signaling pathway activator is selected from the group consisting of retinoic acid, all-trans retinoic acid, 9-cis retinoic acid, CD437, EC23, BS 493, TTNPB, and AM580. In any of the embodiments, the RA signaling pathway activator is RA. In any of the embodiments, the RA signaling pathway activator is contacted at a concentration of 1, 1.1, 1.2, 1.3, 1.4, 1.5, 1.6, 1.7, 1.8, 1.9, 2, 2.1, 2.2, 2.3, 2.4, 2.5, 2.6, 2.7, 2.9, or 3 μM, or about 1, 1.1, 1.2, 1.3, 1.4, 1.5, 1.6, 1.7, 1.8, 1.9, 2, 2.1, 2.2, 2.3, 2.4, 2.6, 2.7, 2.9, or 3 μM, or any concentration within a range defined by any two of the aforementioned concentrations. In any of the embodiments, the RA signaling pathway activator is contacted at a concentration of 2 μM or about 2 μM.

In any of the embodiments, the Wnt signaling pathway activator is selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, alaisine, indirubin, alsterpaullone, kenpaullone, lithium chloride, TDZD 8, and TWS119. In any of the embodiments, the Wnt signaling pathway activator is CHIR99021. In any of the embodiments, the Wnt signaling pathway activator is contacted at a concentration of 0.01, 0.1, 1, 2, 3, 4, 5, 6, 7, 8, 9, 10, 11, 12, 13, 14, 15, 16, 17, 18, 19, or 20 μM, or about 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations. In any of the embodiments, the Wnt signaling pathway activator is contacted at a concentration of 1, 2, 3, 4, 5, 6, 7, 8, 9, or 10 μM, or about 1, 2, 3, 4, 5, 6, 7, 8, 9, or 10 μM, or any concentration within a range defined by any two of the aforementioned concentrations.

In any of the embodiments, the HH signaling pathway activator is selected from the group consisting of SHH, IHH, DHH, PMA, GSA 10, and SAG. In any of the embodiments, the HH signaling pathway activator is PMA. In any of the embodiments, the HH signaling pathway activator is contacted at a concentration of 1, 1.1, 1.2, 1,3, 1.4, 1,5, 1.6, 1.7, 1,8, 1.9, 2, 2.1, 2.2, 2.3, 2.4, 2,5, 2.6, 2.7, 2.8, 2.9, or 3 μM or about 1, 1.1, 1.2, 1,3, 1.4, 1.5, 1.6, 1.7, 1.8, 1,9, 2, 2.1, 2,2, 2.3, 2.4, 2.5, 2.6, 2,7, 2,8, 2.9, or 3 μM, or any concentration within a range defined by any two of the aforementioned concentrations. In any of the embodiments, the HH signaling pathway activator is contacted at a concentration of 2 μM or about 2 μM.

In any of the embodiments, the BMP signaling pathway inhibitor is selected from the group consisting of Noggin, RepSox, LY364947, LDN193189, and SB431542. In any of the embodiments, the BMP signaling pathway inhibitor is Noggin. In any of the embodiments, the BMP signaling pathway inhibitor is contacted at a concentration of 50, 60, 70, 80, 90, 100, 110, 120, 130, 140, or 150 ng/mL or about 50, 60, 70, 80, 90, 100, 110, 120, 130, 140, or 150 ng/mL, or any concentration within a range defined by any two of the aforementioned concentrations. In any of the embodiments, the BMP signaling pathway inhibitor is contacted at a concentration of 100 ng/mL or about 100 ng/mL.

Also disclosed herein are the splanchnic mesoderm cells, septum transversum cells, fibroblasts, respiratory mesenchyme cells, and the esophageal/gastric mesenchyme cells produced by any of the methods disclosed herein.

Embodiments of the present disclosure provided herein are described by way of the following numbered alternatives:

1. A method of producing splanchnic mesoderm cells, comprising:

contacting lateral plate mesoderm cells with a TGF-beta signaling pathway inhibitor, a Wnt signaling pathway inhibitor, a BMP signaling pathway activator, an FGF signaling pathway activator, and a retinoic acid (RA) signaling pathway activator.

2. The method of alternative 1, wherein the splanchnic mesoderm cells are human splanchnic mesoderm cells.

3. The method of alternative 1-2, wherein the lateral plate mesoderm cells have been differentiated from middle primitive stream cells.

4. The method of alternative 3, wherein the lateral plate mesoderm cells have been differentiated from middle primitive streak cells by contacting the middle primitive streak cells with a TGF-beta signaling pathway inhibitor, a Wnt signaling pathway inhibitor, and a BMP signaling pathway activator.

5. The method of alternative 3 or 4, wherein the middle primitive streak cells have been differentiated from pluripotent stem cells.

6. The method of alternative 5, wherein the middle primitive streak cells have been differentiated from pluripotent stem cells by contacting the pluripotent stem cells with a TGF-beta signaling pathway activator, a Wnt signaling pathway activator, an FGF signaling pathway activator, a BMP signaling pathway activator, and a PI3K signaling pathway inhibitor.

7. The method of any one of alternatives 1-6, wherein the lateral plate mesoderm cells are contacted with A8301, BMP4, C59, FGF2, RA, or any combination thereof.

8. The method of any one of alternatives 1-7, wherein the lateral plate mesoderm cells are contacted for a time that is sufficient to differentiate lateral plate mesoderm cells to splanchnic mesoderm cells, and/or for a time that is or is about 36, 37, 38, 39, 40, 41, 42, 43, 44, 45, 46, 47, 48, 49, 50, 51, 52, 53, 54, 55, 56, 57, 58, 59, or 60 hours, or any time within a range defined by any two of the aforementioned times.

9. The method of any one of alternatives 1-8, wherein the lateral plate mesoderm cells are contacted for a time that is or is about 48 hours.

10. The method of any one of alternatives 1-9, wherein the splanchnic mesoderm cells exhibit increased expression of FOXF1, HOXA1, HOXA5, or WNT2, or any combination thereof, and decreased expression of NKX2-5, ISL1, or TBX2, or any combination thereof, relative to cardiac mesoderm cells.

11. The method of any one of alternatives 1-10, wherein the splanchnic mesoderm cells exhibit decreased expression of PAX3 or PRRX1, or both, relative to middle primitive streak cells, and/or decreased expression of CD31 relative to cardiac mesoderm cells.

12. A method of producing septum transversum cells, comprising contacting splanchnic mesoderm cells with a retinoic acid signaling pathway activator and a BMP signaling pathway activator.

13. The method of alternative 12, wherein the splanchnic mesoderm cells are the splanchnic mesoderm cells of any one of alternatives 1-11.

14. The method of alternative 12 or 13, wherein the splanchnic mesoderm cells are contacted with RA, BMP4, or both.

15. The method of any one of alternatives 12-14, wherein the splanchnic mesoderm cells are contacted for a time that is sufficient to differentiate splanchnic mesoderm cells to septum transversum cells, and/or for a time that is or is about 60, 61, 62, 63, 64, 65, 66, 67, 68, 69, 70, 71. 72, 73, 74, 75, 76, 77, 78, 79, 80, 81, 82, 83, or 84 hours, or any period of time within a range defined by any two of the aforementioned times.

16. The method of any one of alternatives 12-15, wherein the splanchnic mesoderm cells are contacted for a period of time that is or is about 72 hours.

17. The method of any one of alternatives 12-16, wherein the septum transversum cells exhibit increased expression of WT1, TBX18, LHX2, UPK3B, or UPK1B, or any combination thereof, relative to cardiac mesoderm cells, splanchnic mesoderm cells, or fibroblasts, or any combination thereof.

18. The method of any one of alternatives 12-17, wherein the septum transversum cells exhibit decreased expression of MSX1, MSX2, or HAND1, or any combination thereof, relative to cardiac mesoderm cells or fibroblasts, or both.

19. The method of any one of alternatives 12-18, wherein the septum transversum cells exhibit decreased expression of HOXA1 or TBX5, or both, relative to splanchnic mesoderm cells.

20. The method of any one of alternatives 12-19, wherein the septum transversum cells exhibit decreased expression of NKX6.1 or HOXA5, or both, relative to respiratory mesenchyme cells.

21. The method of any one of alternatives 12-20, wherein the septum transversum cells exhibit decreased expression of NKX3.2, MSC, BARX1, WNT4, or HOXA5, or any combination thereof, relative to esophageal/gastric mesenchyme cells.

22. The method of any one of alternatives 12-21, wherein the septum transversum cells account for about 60%, 65%, 70%, 75%, 80%, 85%, or 90% of the total cells differentiated from the splanchnic mesoderm cells.

23. A method of producing fibroblasts, comprising contacting splanchnic mesoderm cells with a retinoic acid signaling pathway activator, a BMP signaling pathway activator, and a Wnt signaling pathway activator.

24. The method of alternative 23, wherein the splanchnic mesoderm cells are the splanchnic mesoderm cells of any one of alternatives 1-11.

25. The method of alternative 23 or 24, wherein the splanchnic mesoderm cells are contacted with RA, BMP4, CHIR99021, or any combination thereof.

26. The method of any one of alternatives 23-25, wherein the fibroblasts are liver fibroblasts.

27. The method of any one of alternatives 23-26, wherein the splanchnic mesoderm cells are contacted for a time that is sufficient to differentiate splanchnic mesoderm cells to fibroblasts, and/or for a time that is or is about 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times.

28. The method of any one of alternatives 23-27, wherein the splanchnic mesoderm cells are contacted for a period of time that is or is about 72 hours.

29. The method of any one of alternatives 23-28, wherein the fibroblasts exhibit increased expression of MSX1, MSX2, or HAND1, or any combination thereof, relative to splanchnic mesoderm cells or septum transversum cells, or both.

30. The method of any one of alternatives 23-29, wherein the fibroblasts exhibit decreased expression of WT1, TBX18, HX2, or UPK1B, or any combination thereof, relative to septum transversum cells.

31. The method of any one of alternatives 23-30, wherein the fibroblasts exhibit decreased expression of NKX6.1, HOXA5, or LHX2, or any combination thereof, relative to respiratory mesenchyme cells.

32. The method of any one of alternatives 23-31, wherein the fibroblasts exhibit decreased expression of NKX3.2, MSC, BARX1, WNT4, or HOXA5, or any combination thereof, relative to esophageal/gastric mesenchyme cells.

33. A method of producing respiratory mesenchyme cells, comprising a) contacting splanchnic mesoderm cells with a retinoic acid signaling pathway activator, a BMP signaling pathway activator, a hedgehog (HH) signaling pathway activator, and a Wnt signaling pathway activator.

34. The method of alternative 33, wherein the splanchnic mesoderm cells are contacted for a time that is sufficient to differentiate splanchnic mesoderm cells to respiratory mesenchyme cells, and/or for a time that is or is about 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times.

35. The method of alternative 33 or 34, wherein the splanchnic mesoderm cells are contacted for a period of time that is or is about 72 hours.

36. The method of alternative 33, wherein step a) is a second step, and further comprising a first step of contacting the splanchnic mesoderm cells with a retinoic acid signaling pathway activator, a BMP signaling pathway activator, and a HH signaling pathway activator prior to the second step.

37. The method of alternative 36, wherein the splanchnic mesoderm cells are contacted for a time that is sufficient to differentiate splanchnic mesoderm cells to respiratory mesenchyme cells, and/or for a time or is about 36, 37, 38, 39, 40, 41, 42, 43, 44, 45, 46, 47, 48, 49, 50, 51, 52, 53, 54, 55, 56, 57, 58, 59, or 60 hours, or any period of time within a range defined by any two of the aforementioned times for the first step.

38. The method of alternative 36 or 37, wherein the splanchnic mesoderm cells are contacted for a period of tune that is or is about 48 hours for the first step.

39. The method of any one of alternatives 36-38, wherein the splanchnic mesoderm cells are contacted for a time that is sufficient to differentiate splanchnic mesoderm cells to respiratory mesenchyme cells, and/or for a time or is about 12, 13, 14, 15, 16, 17, 18, 19, 20, 21, 22, 23, 24, 25, 26, 27, 28, 29, 30, 31, 32, 33, 34, 35, or 36 hours, or any period of time within a range defined by any two of the aforementioned times for the second step.

40. The method of any one of alternatives 36-39, wherein the splanchnic mesoderm cells are contacted for a period of time that is or is about 24 hours for the second step.

41. The method of any one of alternatives 33-40, wherein the splanchnic mesoderm cells are the splanchnic mesoderm cells of any one of alternatives 1-11.

42. The method of any one of alternatives 33-41, wherein the splanchnic mesoderm cells are contacted with RA, BMP4, PMA, CHIR99021, or any combination thereof.

43. The method of any one of alternatives 33-42, wherein the respiratory mesenchyme cells exhibit increased expression of NKX6-1, TBX5, HOXA1, HOXA5, FOXF1, LHX2, or WNT2, or any combination thereof, relative to cardiac endoderm cells, splanchnic mesoderm cells, or esophageal/gastric mesenchyme cells, or any combination thereof.

44. The method of any one of alternatives 33-43, wherein the respiratory mesenchyme cells exhibit decreased expression of WNT2, WT1, TBX18, LHX2, or UPK1B, or any combination thereof, relative to septum transversum cells.

45. The method of any one of alternatives 33-44, wherein the respiratory mesenchyme cells exhibit decreased expression of WNT2, MSX1, or MSX2, or any combination thereof, relative to fibroblast cells.

46. A method of producing esophageal/gastric mesenchyme cells, comprising a) contacting splanchnic mesoderm cells with a retinoic acid signaling pathway activator, a BMP signaling pathway inhibitor, and a HH signaling pathway activator.

47. The method of alternative 46, wherein the splanchnic mesoderm cells are contacted for a time that is sufficient to differentiate splanchnic mesoderm cells to esophageal/gastric mesenchyme cells, and/or for a time or is about 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times.

48. The method of alternative 46 or 47, wherein the splanchnic mesoderm cells are contacted for a period of time that is or is about 72 hours.

49. The method of alternative 46, wherein step a) is a second step, and further comprising a first step of contacting the splanchnic mesoderm cells with a retinoic acid signaling pathway activator and a HH signaling pathway activator prior to the second step.

50. The method of alternative 49, wherein the splanchnic mesoderm cells are contacted for a time that is sufficient to differentiate splanchnic mesoderm cells to esophageal/gastric mesenchyme cells, and/or for a time or is about 36, 37, 38, 39, 40, 41, 42, 43, 44, 45, 46, 47, 48, 49, 50, 51, 52, 53, 54, 55, 56, 57, 58, 59, or 60 hours, or any period of time within a range defined by any two of the aforementioned times for the first step.

51. The method of alternative 49 or 50, wherein the splanchnic mesoderm cells are contacted for a period of time that is or is about 48 hours for the first step.

52. The method of any one of alternatives 49-51, wherein the splanchnic mesoderm cells are contacted for a time that is sufficient to differentiate splanchnic mesoderm cells to esophageal/gastric mesenchyme cells, and/or for a time or is about 12, 13, 14, 15, 16, 17, 18, 19, 20, 21, 22, 23, 24, 25, 26, 27, 28, 29, 30, 31, 32, 33, 34, 35, or 36 hours, or any period of time within a range defined by any two of the aforementioned times for the second step.

53. The method of any one of alternatives 49-52, wherein the splanchnic mesoderm cells are contacted for a period of time that is or is about 24 hours for the second step.

54. The method of alternative 46-53, wherein the splanchnic mesoderm cells are the splanchnic mesoderm cells of any one of alternatives 1-11.

55. The method of any one of alternatives 46-54, wherein the splanchnic mesoderm cells are contacted with RA, Noggin, PMA, or any combination thereof.

56. The method of any one of alternatives 46-55, wherein the esophageal/gastric mesenchyme cells exhibit increased expression of MSC, BARX1, WNT4, HOXA1, FOXF1, or NKX3-2, or any combination thereof, relative to cardiac endoderm cells, splanchnic mesoderm cells, or respiratory mesenchyme cells, or any combination thereof.

57. The method of any one of alternatives 46-56, wherein the esophageal/gastric mesenchyme cells exhibit decreased expression of WNT2, TBX5, MSX1, MSX2, or LHX2, or any combination thereof, relative to septum transversum cells, fibroblasts, or respiratory mesenchyme cells, or any combination thereof.

58. The method of any one of alternatives 1-57, wherein the TGF-beta signaling pathway inhibitor is selected from the group consisting of A8301, RepSox, LY365947, and SB431542.

59. The method of any one of alternatives 1-58, wherein the TGF-beta signaling pathway inhibitor is A8301.

60. The method of any one of alternatives 1-59, wherein the TGF-beta signaling pathway inhibitor is contacted at a concentration of 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM or any concentration within a range defined by any two of the aforementioned concentrations.

61. The method of any one of alternatives 1-60, wherein the TGF-beta signaling pathway inhibitor is contacted at concentration of 1 μM or about 1 μM.

62. The method of any one of alternatives 1-61, wherein the Wnt signaling pathway inhibitor is selected from the group consisting of C59, PNU 74654, KY-02111, PRI-724, FH-535 DIF-1, and XAV939.

63. The method of any one of alternatives 1-62, wherein the Wnt signaling pathway inhibitor is C59.

64. The method of any one of alternatives 1-63, wherein the Wnt signaling pathway inhibitor is contacted at a concentration of 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM or about 0.1, 0.2, 0.3, 0.4, 0.5, 0.6, 0.7, 0.8, 0.9, 1, 1.1, 1.2, 1.3, 1.4, 1.5, 1.6. 1.7, 1.8, 1.9, or 2 μM, or any concentration within a range defined by any two of the aforementioned concentrations.

65. The method of any one of alternatives 1-64, wherein the Wnt signaling pathway inhibitor is contacted at a concentration of 1 μM or about 1 μM.

66. The method of any one of alternatives 1-65, wherein the BMP signaling pathway activator is selected from the group consisting of BMP1, BMP2, BMP3, BMP4, BMP5, BMP6, BMP7, BMP8a, BMP8b, BMP10, BMP11, BMP15, IDE1, and IDE2.

67. The method of any one of alternatives 1-66, wherein the BMP signaling pathway activator is BMP4.

68. The method of any one of alternatives 1-67, wherein the BMP signaling pathway activator is contacted at a concentration of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/mL or about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/mL, or any concentration within a range defined by any two of the aforementioned concentrations.

69. The method of any one of alternatives 1-68, wherein the BMP signaling pathway activator is contacted at a concentration of 30 ng/mL or about 30 ng/mL.

70. The method of any one of alternatives 1-69, wherein the FGF signaling pathway activator is selected from the group consisting of FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, and FGF23.

71. The method of any one of alternatives 1-70, wherein the FGF signaling pathway activator is FGF2.

72. The method of any one of alternatives 1-71, wherein the FGF signaling pathway activator is contacted at a concentration of 5, 6, 7, 8, 9,10, 11, 12, 13, 14, 15, 16, 17, 18, 19. 20, 21, 22, 23, 24, 25, 26, 27, 28, 29, 30, 31, 32, 33, 34, or 35 ng/mL, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 ng/mL, or any concentration within a range defined by any two of the aforementioned concentrations.

73. The method of any one of alternatives 1-72, wherein the FGF signaling pathway activator is contacted at a concentration of 20 ng/mL, or about 20 ng/mL.

74. The method of any one of alternatives 1-73, wherein the RA signaling pathway activator is selected from the group consisting of retinoic acid, all-trans retinoic acid, 9-cis retinoic acid, CD437, EC23, BS 493, TTNPB, and AM580.

75. The method of any one of alternatives 1-74, wherein the RA signaling pathway activator is RA.

76. The method of any one of alternatives 1-75, wherein the RA signaling pathway activator is contacted at a concentration of 1, 1.1, 1.2, 1.3, 1.4, 1.5, 1.6, 1.7, 1.8, 1.9, 2, 2.1, 2.2, 2.3, 2.4, 2.5, 2.6, 2.7, 2.9, or 3 μM, or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.9, or 3 μM, or any concentration within a range defined by any two of the aforementioned concentrations.

77. The method of any one of alternatives 1-76, wherein the RA signaling pathway activator is contacted at a concentration of 2 μM or about 2 μM.

78. The method of any one of alternatives 1-77, wherein the Wnt signaling pathway activator is selected from the group consisting of Wnt1, Wnt2 Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BMT 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, and TWS 119.

79. The method of any one of alternatives 1-78, wherein the Wnt signaling pathway activator is CHIR99021.

80. The method of any one of alternatives 1-79, wherein the Wnt signaling pathway activator is contacted at a concentration of 0.01, 0.1, 1, 2, 3, 4, 5, 6, 7, 8, 9, 10, 11, 12, 13, 14, 15, 16, 17, 18, 19, or 20 μM, or about 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations.

81. The method of any one of alternatives 1-80, wherein the Wnt signaling pathway activator is contacted at a concentration of 1, 2, 3, 4, 5, 6, 7, 8, 9, or 10 or about 1, 2, 3, 4, 5, 6, 7, 8, 9, or 10 ΞM, or any concentration within a range defined by any two of the aforementioned concentrations.

82. The method of any one of alternatives 1-81, wherein the signaling pathway activator is selected from the group consisting of SHH, IHH, DHH, PMA, GSA 10, and SAG.

83. The method of any one of alternatives 1-82, wherein the HH signaling pathway activator is PMA.

84. The method of any one of alternatives 1-83, wherein the HH signaling pathway activator is contacted at a concentration of 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 or any concentration within a range defined by any two of the aforementioned concentrations.

85. The method of any one of alternatives 1-84, wherein the HH signaling pathway activator is contacted at a concentration of 2 μM or about 2 μM.

86. The method of any one of alternatives 1-85, wherein the BMP signaling pathway inhibitor is selected from the group consisting of Noggin, RepSox, LY364947, LDN193189, and SB431542.

87. The method of any one of alternatives 1-86, wherein the BMP signaling pathway inhibitor is Noggin.

88. The method of any one of alternatives 1-87, wherein the BMP signaling pathway inhibitor is contacted at a concentration of 50, 60, 70, 80, 90, 100, 110, 120, 130, 140, or 150 ng/mL or about 50, 60, 70, 80, 90, 100, 110, 120, 130, 140, or 150 ng/mL, or any concentration within a range defined by any two of the aforementioned concentrations.

89. The method of any one of alternatives 1-88, wherein the BMP signaling pathway inhibitor is contacted at a concentration of 100 ng/mL or about 100 ng/mL.

90. The splanchnic mesoderm cells produced by the method of any one of alternatives 1-11.

91. The septum ansversum cells produced by the method of any one of alternatives 12-22.

92. The fibroblasts produced by the method of any one of alternatives 23-32.

93. The respiratory mesenchyme cells produced by the method of any one of alternatives 33-45.

94. The esophageal/gastric mesenchyme cells produced by the method of any one of alternatives 46-57.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIGS. 1A-J depicts an embodiment of single cell analysis of the mouse foregut endoderm and mesoderm lineages. FIG. 1A shows representative mouse embryo images at three developmental stages showing the foregut region (dashed) that was micro-dissected (insets) to generate single cells. At E9.5, anterior foregut (a.fg) and posterior foregut (p.fg) were isolated separately. E, embryonic day; s, somite number; n, number of cells. Scale bar 1 mm. FIG. 1B shows a schematic of the RNA-seq workflow. FIG. 1C shows UMAP visualization of 31,268 cells isolated from pooled samples of all three stages. Cells are shaded based on major cell lineages. FIG. 1D shows whole-mount immunostaining of an E9.5 mouse foregut, showing the Cdh1+ endoderm and the surrounding Foxf1+ splanchnic mesoderm. FIGS. 1E and 1F show t-SNE plots of in silico isolated E9.5 endodermal cells (1E) and splanchnic mesodermal (1F) cells. FIGS. 1G and 1H show pseudo-spatial ordering of E9.5 endodermal (1G) and mesodermal (1H) cells along the anterior-posterior (A-P) axis. FIGS. 1I and 1J show schematics of the predicted locations of E9.5 cell types mapped onto the embryonic mouse foregut endoderm (1I) and mesoderm (1J). def, definitive; meso, mesoderm; lg, lung; eso, esophagus; lv, liver; splanch, splanchnic; stm, septum transversum mesenchyme; sto, stomach; pha, pharynx.

FIG. 1K depicts an embodiment of the definition of major cell lineages. UMAP of single cells from all stages with major lineage annotated by known marker genes (panel A). UMAP of all cells from all stages with computationally assigned clusters based on transcriptome similarity (panel B). UMAP of all cells from all stages shaded by stages and regions (panel C). t-SNE maps of single cells from each stage annotated by major lineages at E8.5 (panel D), E9.0 (panel E), and E9.5 (panel F). Gene expression heatmap of selected markers in individual cells across different lineages and stages (panel G).

FIG. 1L depicts an embodiment of annotation of E8.5 and E9.0 DE and SM lineages. t-SNE plots of E8.5 DE (panel A), E8.5 SM (panel B), E9.0 DE (panel C), and E9.0 SM cells (panel D) annotations. E8.5 clusters are designated as ‘a’, E9.0 as ‘b’, and E9.5 as ‘c’. Pseudo-spatial ordering of E8.5 DE (panel E), E8.5 SM (panel F), E9.0 DE (panel G), and E9.0 SM cells (panel H) along the anterior-posterior (A-P) axis of the gut tube. Schematics of the mouse embryonic foregut showing the predicted location of E8.5 DE (panel I), E8.5 SM (panel J), E9.0 DE (panel K), and E9.0 SM (panel L) cell types mapped onto the endoderm and mesoderm. Heatmap of selected marker gene expression in individual cells across different clusters at E8.5 DE (panel M), E8.5 SM (panel N), E9.0 DE (panel O) and E9.0 SM (panel P).

FIG. 1M depicts an embodiment of integrated analysis of DE and SM cells. t-SNE and UMAP visualization of all SM cells from all stages annotated by major lineages (panels A, B) and stages (panels C, D). t-SNE and UMAP visualization of all DE cells from all stages annotated by major lineages (panels E, F) and stage (panels G, H). The stage-specific annotations making major contributions to each integrated cluster are indicated in brackets. E8.5 cells=a_clusters, E9.0 cells=b_clusters, and E9.5=c_clusters.

FIGS. 2A-Q depict an embodiment of lineage-restricted gene expression in different SM cell types. FIG. 2A shows a schematic of the E9.5 foregut indicating the level of sections. FIG. 2B shows a dot plot showing scRNA-seq expression of marker genes in different E9.5 SM cell clusters. FIG. 2C shows whole-mount immunostaining of dissected E9.5 foregut tissue. FIGS. 2D-G show in situ hybridization of dissected E9.5 foregut tissue. Scale bar is 100 μm. FIGS. 2H-2Q show RNA-scope in situ detection on transverse E9.5 mouse embryo sections (i-iv indicates the A-P level of the section in FIG. 2A). Scale bar is 50 μm. duo, duodenum; dp, dorsal pancreas; eso, esophagus; ht, heart; Ig, lung; liv, liver; oft, outflow tract; pha, pharynx; res, respiratory; stm, septum transversum mesenchyme; sto, stomach; sv, sinus venosus; vp, ventral pancreas.

FIG. 2R depicts an embodiment of validation of liver mesenchyme subtypes. Schematic of mouse embryonic foregut at E9.5 (panel A). RNA-scope in situ detection of mesoderm markers on fixed frozen sagittal sections from E9.5 mouse embryos (panels B-F). Scale bar is 50 μm. Insets show merge and separate channels.

FIG. 2S depicts an embodiment of co-linear Hox gene expression and transcription factor code. Heattnaps of average Hox gene expression across different DE and SM clusters arranged along the A-P axis. Annotations are: E8.5=a_clusters, E9.0=b_clusters, E9.5=c_clusters (panel A). Inferred location of cell clusters in the foregut endoderm and mesoderm (panel B). Transcription factor code heattnap showing the average expression of top five distinguishing transcription factor differential expressions across E9.5 DE and SM populations (panel C). a, anterior; fg, foregut; post, posterior; v, ventral; stm, septum transversum mesenchyme.

FIGS. 3A-F depict an embodiment of coordinated endoderm and mesoderm cell trajectories. FIGS. 3A and 3B show force directed SPRING visualization of the splanchnic mesoderm (3A; n=10.097) and definitive endoderm (3B; n=4,448) cell trajectories. Cells are shaded by developmental stage. White arrows indicate cell lineage progression. FIGS. 3C and 3D show a confusion matrix summarizing “parent-child” single cell voting for SM (3C) and DE (3D) cells used to construct the cell state tree. Each cell at a later time point (y-axis) voted for its most similar cell at the preceding time point (x-axis) based on transcriptome similarity (KNN). All the votes for a given cluster are tabulated, normalized for cluster size and represented as a % of votes in the heatmap. E8.5, E9.0 and E9.5 clusters are designated as ‘a’, ‘b’, and ‘c’, respectively. FIGS. 3E and 3F show cell state trees of SM (3E) and DE (3F) lineages predicted by single cell voting. The top choice linking cell states of sequential time points are solid lines, with prominent second choices are dashed lines. Nodes are shaded by stages and annotated with the cluster numbers.

FIG. 3G depicts an embodiment of SPRING plots of DE and SM cell trajectories. SPRING plots of all SM cells (n=10.097) shaded by stage specific lineage annotations (panel A) and expression of key marker genes (panel B). SPRING plots of all DE cells (n=4,448) shaded by stage specific lineage annotations (panel C) and expression of key marker genes (panel D).

FIG. 3H depicts an embodiment of hepatic endoderm development. Cell state tree of the hepatic endodermal lineage with key marker genes indicated in each cell state (panel A). Psuedotime analysis of the hepatic DE lineage using Monocle_v3 suggests that at E9.0, the e_b2 cluster (early hepatoblasts) is a common progenitor of e_b5 (later hepatoblasts) and e_b7 (hepatopancreatic duct progenitors (panel B). SPRING plots with hepatic endodermal clusters shaded by stage specific lineage annotations (panel C) and expression of key marker genes (panels D-I).

FIGS. 4A-I, 4K-L, depict an embodiment of coordinated development of multipotent progenitors. FIGS. 4A, 4B show graphical illustrations of the esophageal-respiratory-gastric cell state trajectories for SM (4A) and DE (4B) with key marker genes. This suggests the coordinated development of Osr1+multi-lineage progenitors. FIGS. 4C and 4D show SPRING plots of SM (4C) and DE (4E) projecting the expression of key genes. FIG. 4E show in situ hybridization of Osr1+ in dissected foregut, showing Osr1 is expressed in the respiratory, esophageal, and gastric regions. FIGS. 4F and 4G show in situ hybridization of Osr1 in sections across the respiratory and gastric regions within the foregut, showing that Osr1 is expressed in both the endodermal and mesenchymal cells. FIG. 41-1 shows a SPRING plot of the DE esophageal-respiratory lineages. FIG. 4I shows Nkx2-1 and Sox2 expression projected onto the SPRING plot, showing co-expression at the esophageal-tracheal boundary. FIG. 4K shows Sox2 and Nkx2-1 whole mount immunostaining of a E9.5 mouse foregut. FIG. 4L show Sox2, Nkx2-1 and Foxf1 immunostaining of a transverse E9.5 foregut section, confirming a rare population of Sox2/Nkx2-1 co-expressing cells. L′ depicts a higher magnification of the box in FIG. 4L.

FIGS. 5A-I depict an embodiment of computationally inferred receptor-ligand interactions predicting a signaling roadmap of foregut organogenesis. FIGS. 5A, 5B show E9.5 foregut immunostaining of Cdh1 (epithelium) and Foxf1 (mesenchyme) in whole mount (5A; same image as 1D) and section (5B), showing the epithelial mesenchyme tissue microenvironment (dashed circle). FIG. 5C shows predicted receptor-ligand interactions between adjacent foregut cell populations. The schematics show paracrine signaling between the DE and SM for six major pathways. E9.5 DE and SM cell clusters are ordered along the anterior to posterior axis based on their locations in vivo, with spatially adjacent DE and SM cell types across from one another. Shaded circles indicate the relative pathway response-metagene expression levels, predicting the likelihood that a given cell population is responding to the growth factor signal. Thin vertical lines next to the clusters indicate different cell populations in spatial proximity that are all responding to a particular signal pathway. Arrows represent the predicted paracrine and autocrine receptor-ligand interactions. FIG. 5D shows BMP response-metagene expression levels projected on the DE and SM SPRING plot. FIG. 5E shows in situ hybridization of Bmp4 in a foregut transverse section, showing the expression in the respiratory mesenchyme and the stm. FIGS. 5F and 5G show pSmad1 immunostaining in foregut transverse sections, indicating BMP signal response in the respiratory and liver DE and SM. FIGS. 5H. and SI show signaling roadmaps summarizing the inferred signaling state of all 6 pathways projected on the DE (5H) and SM (5I) cell state trees suggesting the combinatorial signals predicted to control lineage diversification. The letters indicate the putative signals at each step, with larger font indicating a stronger signaling response. a, anterior; p, posterior; hp, hepatopancreatic; sttn, septum transversum mesenchyme.

FIG. 5J depicts an embodiment of metagene expression for all ligands, receptors and context-independent response genes. Dot plot showing the average scaled expression (2 to −2) of metagenes (X-axis)in each DE and SM cluster (Y-axis). For each cell signaling pathway (BMP, FGF, FIR, Notch, RA, and canonical Wnt), a “ligand-metagene”, “receptor-metagene”, and “response-metagene” was calculated by averaging the normalized expression of each individual gene for each pathway (e.g. Wnt-ligand metagene=ΣWnt1+Wnt2+Wnt2b+Wnt3. . . Wnt10b expression/n) in each cell and cluster. Shading and size of each dot represents the metagene expression level in each cluster.

FIG. 5K depicts an embodiment of computationally predicted receptor-ligand interactions between different foregut cell populations. The schematics show paracrine signaling between the DE and SM for six major pathways. Below the schematics, DE and SM cell clusters of each stage are ordered along the A-P axis consistent with their location in vivo. Spatially adjacent DE and SM cell types are across from one another. Shaded circles for each cluster indicate the likelihood that the cell population is responding to the signal based on the pathway response metagene expression levels. Arrows represent the predicted source of the ligands showing paracrine and autocrine receptor-ligand pairs inferred from metagene expression profiles. Receptor-ligand pairing (arrows) were restricted to cell populations in close spatial proximity. Thin vertical lines next to a group of clusters indicate different cell populations in spatial proximity that are all responding similarly,

FIG. 5L depicts an embodiment of predicted temporal and spatial dynamics of signaling responses. Expression levels of the pathway response-metagene projected onto the DE and SM SPRING plots and cell state trees for the BMP (panels A-B), FGF (panels C-D), HH (panels E-F), Notch (panels G-H), RA (panels and canonical Wnt (panels K-L) pathways. This shows how coordinated spatial domains of signaling activity that correspond to cell lineages are predicted to change over 24 hours from E8.5-E9.5.

FIGS. 6A-H depict an embodiment of a genetic test of the signaling roadmap revealing that HH promotes gut tube versus liver mesenchyme, FIGS. 6A, 6B show SPRING visualization of the HH ligand-metagene expression in DE cells (6A) and HH response-metagene expression in SM cells (6B), FIG. 6C shows the HH response-metagene expression projected onto the SM cell state tree showing low HH activity in the liver and pharynx SM but high activity in the gut tube mesenchyme. FIG. 6D shows that Shh is expressed in the gut tube epithelium but not in the hepatic epithelium (outlined). Gli1-lacZ, a HH-response transgene, is active in the gut tube mesenchyme but not in the liver stm. FIG. 6E shows differentially expressed genes between and Gli2−/− Gli3−/−, and Gli2+/− Gli3+/− mouse E9.5 foreguts through bulk RNA sequencing (log2 FC >1, FDR <5%). FIG. 6F shows a heatmap showing average expression of HH/Gli-regulated genes (from FIG. 6E) in E9.5 DE and SM single cell clusters. FIG. 6G shows gene set enrichment analysis (GSEA) revealing specific cell type enrichment of HH/Gli-regulated genes. FIG. 6H shows a schematic of HH activity in the foregut.

FIG. 7A-D depict an embodiment of the generation of splanchnic mesoderm-like progenitors from human PSCs. FIG. 7A shows a schematic of the protocol to differentiate hPSCs into SM subtypes. Factors were predicted from the mouse single cell signaling roadmap. FIG. 7B shows RT-PCR of markers with enriched expression in specific SM subtypes based on the mouse single cell data.: cardiac (NKX2-5), early SM (FOXF1, HOXA1), liver-stm/mesothelium (WT1, UKP1B), liver-fibroblast (MSX1), respiratory SM (NKX6-1+, MSG−), esophageal/gastric (MSC, BARX1). Columns show the means±S.D. Tukey's test *p<0.05, ** p<0.005, ***p<0.0005. FIG. 7C shows immunostaining of Day 7 cell cultures. Scale bar is 50 μm (upper panels), 10 μm (lower panels). FIG. 7D shows quantification of % cells positive for the indicated immunostaining or RNA-scope in situ hybridization. Columns show the means±S.D. (n=3). Tukey's test, *p<0.05, **p<0.005*** p<0.0005.

FIG. 7E depicts an embodiment of data showing that RA suppresses cardiac mesoderm and promotes splanchnic mesoderm progenitors, Staining of RARE-lacZ transgenic mouse embryos confirms the single cell RNA-seq predictions that RA activity is higher in the splanchnic mesenchyme than the cardiac mesenchyme at E8.5 (panel A). Immunostaining of transversal section of RARE-lacZ transgenic mouse embryos (panel B). Day 4 PSC-derived SM cultures assayed by RT-PCR for paraxial mesoderm (PAX3), limb bud (PRIM), cardiac mesoderm (NKX2.5, ISL1), endothelium (CD31), and SM (HOXA1, HOXA5, WNT2) markers; scale bars are 50 μm (panel C), Quantification of NKX2-5+ cells (panel D). fg, foregut; hg, hindgut; ht, heart; SC, stem cell; MPS, middle primitive streak; CM, cardiac mesoderm; SM, splanchnic mesoderm. Columns show the means±S.D. (n=3). Tukey's test *p<0.05, **p<0.005, *** p<0.0005.

FIG. 7F depicts an embodiment of additional analysis of day 7 SM-like PSC cultures. RNA-scope in situ analysis of different d7 SM-like cultures; scale bars are 50 μm for upper panels, 10 μm for lower panels; quantification is in FIG. 7D (panels A-C). RT-PCR analysis of mesoderm subtype markers based on the mouse scRNA-seq data; cardiac (ACTC1, TBX20, TNNT2), early SM (PDE5A, HOXA5); liver-stm/mesothelium (TBX18, LHX2, UPK3B), liver-fibroblast (MSX2, HAND1), esophageal/gastric (WNT4, NKX3-2) (panel D). SC, stem cell; MPS, middle primitive streak; CM, cardiac mesoderm; SM, splanchnic mesoderm; STM, septum transversum mesenchyme; LF, liver fibroblast; RM, respiratory mesenchyme; EM/GM, esophageal/gastric mesenchyme. Columns show the mean±S.D. (n=3). Tukey's test, *p<0.05, **p<0.005, ***p<0.0005.

### DETAILED DESCRIPTION

Visceral organs, such as the lungs, stomach, liver and pancreas, are derived from the fetal foregut through a series of inductive interactions between the definitive endoderm (DE) and the surrounding splanchnic mesoderm (SM). While patterning of DE lineages has been fairly well studied paracrine signaling controlling SM regionalization and how this is coordinated with the epithelial identity during organogenesis is obscure. Disclosed herein are single cell transcriptomics to generate a high-resolution cell state map of the embryonic mouse foregut, This uncovered an unexpected diversity in the SM cells that developed in close register with the organ-specific epithelium. From these data, a spatiotemporal signaling roadmap of the combinatorial endoderm-mesoderm interactions that orchestrate foregut organogenesis was inferred. Key predictions were validated with mouse genetics, showing the importance of endoderm-derived signals in mesoderm patterning. Leveraging the signaling roadmap, different SM subtypes were generated from human pluripotent stem cells (hPSCs), which previously have been elusive.

A critical inductive role for the tnesenchytne in gut tube organogenesis was first established in the 1960s, when it was shown that SM transplanted from different anterior-posterior (A-P) regions of the embryo could instruct the adjacent epithelium to adopt the organ identity consistent with the original SM position. Since then, mesoderm derived paracrine signals in endoderm organogenesis have been examined, but most of these studies have focused on individual organ lineages or individual signaling pathways and therefore lack a comprehensive understanding of the temporally dynamic combinatorial signaling in the foregut microenvironment that orchestrates organogenesis. Moreover, several fundamental questions about the mesoderm remain unanswered over the decades. How many types of SM are there, and does each fetal organ primordia have its own specific mesenchyme? How are the SM and DE lineages coordinated during organogenesis? What role if any does endoderm have in regionalization of the mesoderm.

Initial specification and patterning of the embryonic mesoderm and endoderm occurs during gastrulation, from E6.25 to E8.0 in the mouse, as these germ layers progressively emerge from the primitive streak. The lateral plate mesoderm emerges from the streak after the extra-embryonic mesoderm, and is followed by the intermediate, paraxial and axial mesoderm. Concomitantly, DE cells also delaminate from the streak and migrate along the outer surface of the mesoderm, eventually intercalating into the overlaying visceral endoderm. By E8.0, morphogenetic processes begin to transform the bi-layered sheet of endoderm and mesoderm into a tube structure as the anterior DE folds over to form the foregut diverticulum and the adjacent lateral plate mesoderm containing cardiac progenitors migrates towards the ventral midline. The lateral plate mesoderm further splits into an outer somatic mesoderm layer next to the ectoderm which gives rise to the limbs and body wall, and an inner splanchnic mesoderm layer, which surrounds the epithelial gut tube. The first molecular indication of regional identity in the SM is the differential expression of flax genes along the A-P axis of the embryo. However, in contrast to heart development, where cell diversification has been well studied, the molecular mechanism governing the foregut SM regionalization are obscure particularly during the critical 24 hours when the foregut DE subdivide into distinct organ primordia.

Recently, single cell transcriptomics have begun to examine organogenesis at an unprecedented resolution. However, studies in the developing gut have either primarily examined the epithelial component or later fetal organs after they have been specified. As described herein, single cell transcriptornics of the mouse embryonic foregut was used to infer a comprehensive “cell state” ontogeny of DE and SM lineages, discovering an unexpected diversity in SM progenitor subtypes that develop in close register with the organ-specific epithelium. Projecting the transcriptional profile of paracrine signaling pathways onto these lineages, a roadmap of the reciprocal endoderm-mesoderm inductive interactions that coordinate organogenesis is inferred. Key predictions were validated with mouse genetics showing that differential hedgehog signaling from the epithelium patterns the SM into gut tube mesenchyme versus mesenchyme of the liver. Leveraging the signaling road map, different subtypes of human SM were generated from hPSCs, which have previously been elusive.

As disclosed herein, single cell transcriptomics were used to define the complexity of DE and SM cell types in the embryonic mouse foregut over the first 24 hours of organogenesis as the primitive gut tube is subdivided into distinct organ domains. Herein, an unexpected diversity of distinct cell types in the foregut mesenchyme, defined by new marker genes and a combinatorial code of transcription factors is revealed. Cell trajectories indicate that the development of organ-specific DE and SM is closely coordinated, suggesting to us a tightly regulated signaling network. A putative ligand-receptor signaling roadmap of the reciprocal epithelial-mesenchymal interactions that are likely to coordinate lineage specification of the two tissue compartments were computationally predicted. The disclosure herein represents a valuable resource for further experimentational examination of foregut organogenesis and the data can be explored on the World. Wide Web at research.cchmc.org/ZornLab-singlecell.

Prior studies of SM regional identity in the early embryo have been limited. Besides well-known regionalization of Hox gene expression, most studies have largely focused on individual organs such as the gastric or pulmonary mesenchyme. By comparing single cell transcriptomes across the entire foregut, an extensive regionalization of the early SM into distinct organ-specific mesenchyme subtypes was revealed. It is possible that the divergent transcription signatures of early SM cell types are only transiently utilized to define position and molecular programs during fetal organogenesis. After organ fate is determined, different SM cell types may converge on similar differentiation programs such as smooth muscle or fibroblasts, which are common in all visceral organs. However, the results herein of fetal SM diversification are interesting in light of the emerging idea of organ-specific stromal cells in adults, such as hepatic versus pancreatic stellate cells and pulmonary specific fibroblasts. For example, Tbx4 is expressed in embryonic respiratory SM and later is specifically maintained in adult pulmonary fibroblasts but not in fibroblasts of other organs. Future integrated analyses of the data herein with other single cell RNA sequencing (scRNA-seq) datasets from later fetal and adult organs should resolve how transcriptional programs evolve during cellular differentiation, homeostasis and pathogenesis.

One unexpected observation was that the liver bud contained more distinct SM cell states than any other organ primordia with the septum transversum mesenchyme sinus venosus, two mesothelium and a fibroblast population. This may be due to the fact that unlike other GI organs that form by epithelium evagination, the hepatic endoderm delaminates and invades the adjacent stm, a process that may require more complex epithelial-mesenchymal interactions with the extracellular matrix. Our transcriptome analysis is consistent with lineage tracing experiments showing that the early sun gives rise to the mesothelium, hepatic stellate cells, stromal fibroblasts, and perivascular smooth muscle. It will be important to determine if other organ buds have a similar elaboration of cell types as they differentiate. Alternatively, mesothelium and fibroblasts that originate in the liver may migrate to other organ buds. Indeed, mesenchymal cell movement is one confounding limitation of our study and there is good evidence that mesothelium of the liver bud, also known as the proepicardium, migrates to surround the heart and lungs.

The foregut SM and the cardiac mesoderm are closely related, both arising from the anterior lateral plate mesoderm. A preliminary cross-comparison of the data provided herein with recent single cell RNA-seq studies of the early heart suggests to us that this common origin is reflected in the transcriptomes. The developing heart tube is contiguous with the ventral foregut SM (also known as the second heart field [SHF]), with the arterial pole attached to the pharyngeal SM and the venous pole attached to the lung/liver SM. Fate mapping studies indicate that the second heart field gives rise to heart tissue as well as pharyngeal SM, respiratory SM, and pulmonary vasculature. Indeed, the single cell transcriptomics and genetic analysis of Gli mutants provided herein indicate that the epithelium derived HH signals are critical for the development of these cardio-pulmonary progenitors.

The signaling roadmap developed here was used to direct the development of hPSCs into different SM-like cell types. The system described herein provides a unique opportunity to model human fetal mesenchyme development and to interrogate how combinatorial signaling pathways direct parallel mesenchymal fate choices. The hPSC-derived SM-like tissue produced herein may be used for tissue engineering, drug screening, and personalized medicine. To date, most IiPSC-derived foregut organoids (e.g. gastric, esophageal, pulmonary) tend to lack mesenchyme, unlike hindgut derived intestinal organoids. This is because the conventional differentiation protocols needed to make foregut epithelium is not compatible with mesenchymal development. Therefore, the protocols disclosed herein enable the recombination of DE and SM organoids, an important step towards engineering complex foregut tissue for regenerative medicine.

Disclosed herein are methods of producing splanchnic mesoderm cells in vitro. In some embodiments, the splanchnic mesoderm cells are differentiated from pluripotent stem cells, such as embryonic stem cells or induced pluripotent stem cells. These pluripotent stern cells may be derived from a subject or patient, such that the splanchnic mesoderm cells and any downstream cell types that are produced can be used for various aspects of personalized medicine. These splanchnic mesoderm cells are early progenitor cells during embryogenesis and can be further differentiated into downstream cell types, such as liver, respiratory, esophageal, and/or gastric lineages. The splanchnic mesoderm cells and any downstream cell types also have implications in the production of PSC-derived organoids, which, as stated herein, may lack enough mesenchymal cells such that growth and maturation of the organoids is hindered. The splanchnic mesoderm cells and methods of making the same may be applied to any organoids and/or enteroids (organoid-like structures derived from epithelial tissue and lacking any mesenchyme) described herein or otherwise known in the art. For example, methods of producing organoids or enteroids can be found in U.S. Pat. Nos. 9,719,068 and 10,174,289, and PCT Publications WO 2011/140411, WO 2015/183920, WO 2016/061464, WO 2017/192997, WO 2018/106628, WO 2018/200481, WO 2018/085615, WO 2018/085622, WO 2018/085623, WO 2018/226267, WO 2020/023245, each of which is hereby expressly incorporated by reference in its entirety.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the FIGS., can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g, linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35. 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA). spectroscopy, UV-visible spectrometry, infrared spectrometry, mass spectrometry, nuclear magnetic resonance, gravitnetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “%” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs).” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In sonic embodiments, the precursor cells are subjected to the treatment of external factors (e.g., growth factors) to acquire pluripotency. In some embodiments, a precursor cell can be a totipotent (or omnipotent) stern cell; a pluripotent stem cell (induced or non-induced); a multipotent stein cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stern cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. It would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton Univers and the University of Pennsylvania. Exemplary embryonic stem cells that can be used in embodiments in accordance with the present disclosure include but are not limited to SA01 (SA001); SA02 (SA002); ES01 (TIES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCO1 (HFS11), UC06 (HSF6), WA01 (H1); WA07 (H7); WA09 (H9); WA13 (H13), WA14 (H14). Exemplary human pluripotent cell lines include but are not limited to 72_3, TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), tnitotnycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, Which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

Some embodiments described herein relate to pharmaceutical compositions that comprise, consist essentially of, or consist of an effective amount of a cell composition described herein and a pharmaceutically acceptable carrier, excipient, or combination thereof. A pharmaceutical composition described herein is suitable for human and/or veterinary applications.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a slate government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation typically suits the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylarnines, including methylarnine, ditnethyla.mine, and triethyla.mine; mono-, di-, or trihydroxyalkylatnines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The disclosure herein generally uses affirmative language to describe the numerous embodiments. The disclosure also includes embodiments in which subject matter is excluded, in full or in part, such as substances or materials, method steps and conditions, protocols, or procedures.

### Differentiation of PSCs to Mesoderm

During embryogenesis, the mesoderm is one of the three primary germ layers and gives rise to a wide range of tissues including muscle, connective tissue, bone, cartilage, skin, endothelium, mesenchyme, and blood cells. The mesenchyme that derived from mesoderm have important roles in supporting associated tissue including epithelial tissue for proper growth and development. The mesoderm comprises the paraxial mesoderm, intermediate mesoderm, and the lateral plate mesoderm. The lateral plate mesoderm is further subdivided into the somatic mesoderm and splanchnic mesoderm layers. The splanchnic mesoderm develops intimately with the endoderm and gives rise to many downstream tissue types such as blood vessels, cardiac muscle, and the connective tissue and muscle of the gastrointestinal system. As disclosed herein, the retinoic acid signaling pathway is important for differentiating the lateral plate mesoderm to splanchnic mesoderm.

Any methods for producing any embryonic cell type (e.g. mesoderm endoderm, or ectoderm) from pluripotent stem cells are applicable to the methods described herein. In some embodiments, the pluripotent stem cells are derived from a morula. In some embodiments the pluripotent stem cells are embryonic stem cells or induced pluripotent stem cells. Embryonic stem cells can be derived from the embryonic inner cell mass or from the embryonic gonadal ridges. Embryonic stem cells or induced pluripotent stem cells can originate from a variety of animal species including but not limited to mouse, rat, monkey, cat, dog, hamster, or human. In some embodiments, the embryonic stem cells or the induced pluripotent stem cells are human. In some embodiments, the PSCs are genetically modified such as to express an exogenous nucleic acid or protein, before differentiating to downstream cell types,

In some embodiments, PSCs, such as ESCs and iPSCs, undergo directed differentiation into embryonic germ layer cells, organ tissue progenitor cells, and then into tissue such as gastrointestinal tissue or any other biological tissue. In some embodiments, the directed differentiation is done in a stepwise manner to obtain each of the differentiated cell types where molecules (e.g. growth factors, ligands, agonists, antagonists) are added sequentially as differentiation progresses. In some embodiments, the directed differentiation is done in a non-stepwise manner where molecules (e.g. growth factors, ligands, agonists, antagonists) are added at the same time. In some embodiments, directed differentiation is achieved by selectively activating certain signaling pathways in the PSCs or any downstream cells.

In some embodiments, the signaling pathways include but are not limited to the Wnt signaling pathway; Wnt/APC signaling pathway; FGF signaling pathway; TGF-beta signaling pathway; BMP signaling pathway; Notch signaling pathway; Hedgehog signaling pathway; LKB signaling pathway; PI3K signaling pathway; retinoic acid signaling pathway, ascorbic acid signaling pathway; or Par polarity signaling pathway, or any combination thereof. It will be understood by one of skill in the art that altering the concentration, expression or function of any one of the signaling pathways disclosed herein can drive differentiation in accordance of the present disclosure. In some embodiments, cellular constituents associated with the signaling pathways, for example, natural inhibitors, antagonists, activators, or agonists of the pathways can be used to result in inhibition or activation of the signaling pathways. In some embodiments, siRNA. and/or shRNA targeting cellular constituents associated with the signaling pathways are used to inhibit or activate these pathways.

In some embodiments, pluripotent stem cells, lateral plate mesoderm cells, splanchnic mesoderm cells, or any differentiated cells thereof, are contacted with a Wnt signaling pathway activator or Wnt signaling pathway inhibitor. In some embodiments, the Wnt signaling pathway activator comprises a Wnt protein. In some embodiments, the Wnt protein comprises a recombinant Wnt protein. In some embodiments, the Wnt signaling pathway activator comprises Wnt1, Wnt2 Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, or any combination thereof. In some embodiments, the Wnt signaling pathway activator comprises a GSK3 signaling pathway inhibitor. In some embodiments, the Wnt signaling pathway activator comprises CHIR99021, CHIR 98014, AZD2858, BIO, AR-A014418, SB 216763, SB 415286, aloisine, indirubin, aisterpaulione, kenpaullone, lithium chloride, TDZD 8, or TWS119, or any combination thereof. In some embodiments, the Wnt signaling pathway inhibitor comprises C59, PNU 74654, KY-02111, PRI-724, FH-535, DIF-1, or XAN¹939, or any combination thereof. In some embodiments, the cells are not treated with a Wnt signaling pathway activator or Wnt signaling pathway inhibitor. The Wnt signaling pathway activator or Wnt signaling pathway inhibitor provided herein may be used in combination with any of the other growth factors, signaling pathway activators, or signaling pathway inhibitors provided herein.

Fibroblast growth factors (FGFs) are a family of growth factors involved in angiogenesis, wound healing, and embryonic development. The FGFs are heparin-binding proteins and interactions with cell-surface associated heparan sulfate proteoglycans have been shown to be essential for FGF signal transduction. FGFs are key players in the processes of proliferation and differentiation of wide variety of cells and tissues. In humans, 22 members of the FGF family have been identified, all of which are structurally related signaling molecules. Members FGF1 through FGF10 all bind fibroblast growth factor receptors (FGFRs). FGF1 is also known as acidic fibroblast growth factor, and FGF2 is also known as basic fibroblast growth factor (bFGF). Members FGF11, FGF12, FGF13, and FGF14, also known as FGF homologous factors 1-4 (FHF1-FHF4), have been shown to have distinct functional differences compared to the FGFs. Although these factors possess remarkably similar sequence homology, they do not bind FGFRs and are involved in intracellular processes unrelated to the FGFs. This group is also known as “iFGF.” Members FGF15 through FGF23 are newer and not as well characterized. FGF15 is the mouse ortholog of human FGF19 (hence there is no human FGF15). Human FGF20 was identified based on its homology to Xenopus FGF-20 (XFGF-20). In contrast to the local activity of the other FGFs, FGF15/FGF19, FGF21 and FGF23 have more systemic effects.

In some embodiments, pluripotent stem cells, lateral plate mesoderm cells, splanchnic mesoderm cells, or any differentiated cells thereof, are contacted with an FGF signaling pathway activator. In some embodiments, the FGF signaling pathway activator comprises an FGF protein. In some embodiments, the FGF protein comprises a recombinant FGF protein. In some embodiments, the FGF signaling pathway activator comprises one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, or FGF23. In some embodiments, the cells are not treated with an FGF signaling pathway activator. The FGF signaling pathway activator provided herein may be used in combination with any of the other growth factors, signaling pathway activators, or signaling pathway inhibitors provided herein,

In some embodiments, pluripotent stem cells, lateral plate mesoderm cells, splanchnic mesoderm cells, or any differentiated cells thereof, are contacted with a TGF-beta signaling pathway activator or TGF-beta signaling pathway inhibitor. In some embodiments, the TGF-beta, family comprises bone morphogenetic protein (BMP), growth and differentiation factor (GDF), anti-Millierian hormone, Activin, and Nodal pathways. In some embodiments, the TGF-beta signaling pathway activator comprises TGF-beta 1, TGF-beta 2, TGF-beta 3, Activin A, Activin B, Nodal, a BMP, IDE1A, IDE2, or any combination thereof. In some embodiments, the TGF-beta signaling pathway inhibitor comprises A8301, RepSox, LY365947, SB431542, or any combination thereof. In some embodiments, the cells are not treated with a TGF-beta signaling pathway activator or TGF-beta signaling pathway inhibitor. The TGF-beta signaling pathway activator or TGF-beta signaling pathway inhibitor provided herein may be used in combination with any of the other growth factors, signaling pathway activators, or signaling pathway inhibitors provided herein.

In some embodiments, pluripotent stem cells, lateral plate mesoderm cells, splanchnic mesoderm cells, or any differentiated cells thereof, are contacted with a BMP signaling pathway activator or BMP signaling pathway inhibitor. In some embodiments, the BMP signaling pathway activator comprises a BMP protein. In some embodiments, the BMP protein is a recombinant BMP protein. In some embodiments, the BMP signaling pathway activator comprises BMP1, BMP2, BMP3, BMP4, BMP5, BMP6, BMP7, BMP8a, BMP8b, BMP10, BMP11, BMP15, IDE1 or IDE2, or any combination thereof. In some embodiments, the BMP signaling pathway inhibitor comprises Noggin, RepSox, LY364947, LDN193189, SB431542, or any combination thereof. In some embodiments, the cells are not treated with a BMP signaling pathway activator or BMP signaling pathway inhibitor. The BMP signaling pathway activator or BMP signaling pathway inhibitor provided herein may be used in combination with any of the other growth factors, signaling pathway activators, or signaling pathway inhibitors provided herein.

In some embodiments, pluripotent stem cells, lateral plate mesoderm cells, splanchnic mesoderm cells, or any differentiated cells thereof, are contacted with a Notch signaling pathway activator or Notch signaling pathway inhibitor. In some embodiments, the Notch signaling pathway activator comprises a Notch protein. In some embodiments, the Notch protein comprises a recombinant Notch protein. In some embodiments, the Notch pathway activator comprises JAG1, JAG2, Notch 1, Notch 2, Notch 3, or Notch 4, or any combination thereof. In some embodiments, the Notch pathway inhibitor comprises Compound E, LY411575, DBZ, or DAPT, or any combination thereof. In some embodiments, the cells are not treated with a Notch signaling pathway activator or Notch signaling pathway inhibitor. The Notch signaling pathway activator or Notch signaling pathway inhibitor provided herein may be used in combination with any of the other growth factors, signaling pathway activators, or signaling pathway inhibitors provided herein.

In some embodiments, pluripotent stem cells, lateral plate mesoderm cells, splanchnic mesoderm cells, or any differentiated cells thereof, are contacted with a hedgehog (HH) signaling pathway activator or HH signaling pathway inhibitor. In some embodiments, the HH signaling pathway activator comprises a HH protein. In some embodiments, the HH protein is a recombinant HH protein. In some embodiments, the HH signaling pathway activator comprises SHH, IHH, DHH, purmorphamine (PMA), GSA 10, SAG, or any combination thereof. In some embodiments, the HH signaling pathway inhibitor comprises HPI-1, cyclopamine, GANT 58, or GANT61, or any combination thereof. In some embodiments, the cells are not treated with a HH signaling pathway activator or HH signaling pathway inhibitor. The HH signaling pathway activator or HH signaling pathway inhibitor provided herein may be used in combination with any of the other growth factors, signaling pathway activators, or signaling pathway inhibitors provided herein.

In some embodiments, pluripotent stem cells, lateral plate mesoderm cells, splanchnic mesoderm cells, or any differentiated cells thereof, are contacted with a PI3K signaling pathway activator or PI3K signaling pathway inhibitor. In some embodiments, the PI3K signaling pathway activator comprises 740 Y-P, or erucic acid, or both. In some embodiments, the PI3K signaling pathway inhibitor comprises wortmannin, LY294002, hibiscone C, PI-103, IC-87114, ZSTK474, AS-605240, PIK-75, PIK-90, PIK-294, PIK-293, AZD6482, PF-04691502, GSK1059615, quercetin, pluripotin, flurbiprofen, GDC-0941, dactolisib, pictilisib, idelalisib, buparlisib, rigosertib, copanlisib, duvelisib, alpelisib, or any combination thereof. In some embodiments, the cells are not treated with a PI3K signaling pathway activator or PI3K signaling pathway inhibitor. The PI3K signaling pathway activator or PI3K signaling pathway inhibitor provided herein may be used in combination with any of the other growth factors, signaling pathway activators, or signaling pathway inhibitors provided herein.

In some embodiments, pluripotent stem cells, lateral plate mesoderm cells, splanchnic mesoderm cells, or any differentiated cells thereof, are contacted with a retinoic acid signaling pathway activator or retinoic acid signaling pathway inhibitor. In some embodiments, the retinoic acid signaling pathway activator comprises retinoic acid, all-trans retinoic acid, 9-cis retinoic acid, CD437, EC23, BS 493, TTNPB, or AM580, or any combination thereof. In some embodiments, the retinoic acid signaling pathway inhibitor comprises guggulsterone. In some embodiments, the cells are not treated with a retinoic acid signaling pathway activator or retinoic acid signaling pathway inhibitor. The retinoic acid signaling pathway activator or retinoic acid signaling pathway inhibitor provided herein may be used in combination with any of the other growth factors, signaling pathway activators, or signaling pathway inhibitors provided herein.

In some embodiments, pluripotent stem cells, lateral plate mesoderm cells, splanchnic tnesoderm cells, or any differentiated cells thereof, are contacted with an ascorbic acid signaling pathway activator. In some embodiments, the ascorbic acid signaling pathway activator comprises ascorbic acid or 2-phospho-ascorbic acid, or both. In some embodiments, the cells are not treated with an ascorbic acid signaling pathway activator. The ascorbic acid signaling pathway activator provided herein may be used in combination with any of the other growth factors, signaling pathway activators, or signaling pathway inhibitors provided herein.

In some embodiments, for any of the small molecule compounds, signaling pathway activators, signaling pathway inhibitors, or growth factors, the cells are contacted for a time that is, is about, is at least, is at least about, is not more than, or is not more than about, 1 hour, 2 hours, 3 hours, 4 hours, 5 hours, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 1 hour to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, for any of the small molecule compounds, signaling pathway activators, signaling pathway inhibitors, or growth factors, the cells (e.g. phiripotent stem cells, lateral plate mesoderm cells, splanchnic mesoderm cells, or any differentiated cells thereof) are contacted in culture such that the concentration of any of the small molecule compounds, signaling pathway activators, signaling pathway inhibitors, or growth factors is at a concentration that is, is about, is at least, is at least about, is not more than, or is not more than about, 10 ng/mL, 20 ng/mL, 50 ng/mL, 75 ng/mL, 100 ng/mL, 120 ng/mL, 150 ng/mL, 200 ng/mL, 500 ng/mL, 1000 ng/mL, 1200 ng/mL, 100 ng/mL, 2000 ng/mL, 5000 ng/mL, 7000 ng/mL, 10000 ng/mL, or 15000 ng/mL, or any concentration that is within a range defined by any two of the aforementioned concentrations, for example, 10 ng/mL to 15000 ng/mL, 100 ng/mL to 5000 ng/mL, 500 ng/mL to 2000 ng/mL, 10 ng/mL to 2000 ng/mL, or 1000 ng/mL to 15000 ng/mL. In some embodiments, for any of the small molecule compounds, signaling pathway activators, signaling pathway inhibitors, or growth factors, the cells (e.g. pluripotent stem cells, lateral plate mesoderm cells, splanchnic mesoderm cells, or any differentiated cells thereof) are contacted in culture such that the concentration of any of the small molecule compounds, signaling pathway activators, signaling pathway inhibitors, or growth factors is at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM. In some embodiments, concentration of small molecule compounds, activators, inhibitors, or growth factors is maintained at a constant level throughout the treatment. In some embodiments, concentration of the small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, the cells (e.g. pluripotent stem cells, lateral plate mesoderm cells, splanchnic mesoderm cells, or any differentiated cells thereof) are cultured in growth media that supports the growth of stem cells and differentiated cells thereof. In some embodiments, the growth media is RPMI 1640, DMEM, DMEM/F12, mTeSR1, or mTeSR Plus media. In some embodiments, the growth media comprises fetal bovine serum (FBS), In some embodiments, the growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the growth media does not contain xenogeneic components. In some embodiments, the growth media comprises one or more small molecule compounds, activators, inhibitors, or growth factors.

In some embodiments, pluripotent stem cells are prepared from somatic cells. In some embodiments, pluripotent stem cells are prepared from biological tissue obtained from a biopsy. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, pluripotent stem cells are prepared from PBMCs by viral transduction. In some embodiments, PBMCs are transduced with Sendai virus, lentivirus, adenovirus, or adeno-associated virus, or any combination thereof. In some embodiments, PBMCs are transduced with Sendai virus comprising expression vectors for Oct3/4, Sox2, Klf4, ort,-Myc, or any combination thereof. In some embodiments, PBMCs are transduced with one or more viruses at an MOI that is, is about, is at least, is at least about, is not more than, or is not more than about, 0, 0.1, 0.5, 1.0, 1.5, 2.0, 2.5, 3.0, 3.5, 4.0, 4.5, or 5.0 MOI, or any MOI within a range defined by any two of the aforementioned MOIs, for example, 0 to 5.0, 1.0 to 4.0, 2.0 to 3.0, 0 to 3.0, or 1.0 to 5.0. In some embodiments, after transduction, PBMCs express stem cell reprogramming factors. In some embodiments, after transduction, PBMCs are reprogrammed to iPSCs. In some embodiments, iPSCs are grown on a feeder cell substrate. In some embodiments, iPSCs are grown on a MEF feeder cell substrate. In some embodiments, iPSCs are grown on an irradiated MEF feeder cell substrate. In some embodiments, iPSCs are grown in RPMI 1640, DMEM, DMEM/F12, mTeSR 1, or mTeSR. Plus media,

In some embodiments, PSCs are expanded in cell culture. In some embodiments, iPSCs are expanded in an extracellular matrix, or mimetic or derivative thereof. In some embodiments, the extracellular matrix, or mimetic or derivative thereof, comprises polymers, proteins, polypeptides, nucleic acids, sugars, lipids, poly-lysine, poly-ornithine, collagen, gelatin, fibronectin, vitronectin, laminin, elastin, tenascin, heparan sulfate, entactin, nidogen, osteopontin, basement membrane, Matrigel, Geltrex, hydrogel, PEI, WGA, or hyaluronic acid, or any combination thereof. In some embodiments, PSCs are expanded in Matrigel, Geltrex, or 1% gelatin, or any combination thereof. In some embodiments, PSCs are expanded in cell culture media comprising a ROCK inhibitor (e.g. Y-27632).

### Differentiation to Lateral Plate Mesoderm

Any methods for producing lateral plate mesoderm cells from pluripotent stem cells disclosed herein or otherwise known in the art are applicable to the methods described herein.

In some embodiments, the pluripotent stem cells are first differentiated to middle primitive streak cells. In some embodiments, the pluripotent stem cells are contacted with a TGF-beta signaling pathway activator, a Wnt signaling pathway activator, an FGF signaling pathway activator, a BMP signaling pathway activator, or a PI3K signaling pathway inhibitor, or any combination thereof, to differentiate the PSCs to middle primitive streak cells. In some embodiments, the TGF-beta signaling pathway activator is selected from the group consisting of TGF-beta 1, TGF-beta 2, TGF-beta 3, Activin A, Activin B, Nodal, a BMP, IDE1, and IDE2. In some embodiments, the Wnt signaling pathway activator is selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt16, BML, 284, IQ-1, WAY 262611, CHIR99021, CHIR 98014, AZD2858, BIO, AR-A014418, SB 216763, SB 415286, aloisine, indirubin, aisterpaullone, kenpaullone, lithium chloride, TDZD 8, and TWS119. In some embodiments, the FGF signaling pathway activator is selected from the group consisting of FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, and FGF23. In some embodiments, the BMP signaling pathway activator is selected from the group consisting of BMP1, BMP2, BMP3, BMP4, BMP5, BMP6, BMP7, BMP8a, BMP8b, BMP10, BMP11, BMP15, IDE1, and IDE2. In some embodiments, the PI3K signaling pathway inhibitor is selected from the group consisting of wortmannin. LY294002, hibiscone C, PI-103, IC-87114, ZSTK474, AS-605240, PIK-75, PIK-90, PIK-294, PIK-293, AZD6482, PF-04691502, GSK1059615, quercetin, pluripotin, flurbiprofen, GDC-0941, dactolisib, pictilisib, idelalisib, buparlisib, rigosertib, copanlisib, duvelisib, and alpelisib. In some embodiments, the PSCs are contacted with Activin A, CHIR99021, FGF2, BMP4, or PIK90, or any combination thereof, including all five, to differentiate the PSCs to middle primitive streak cells.

In some embodiments, the PSCs are contacted with a TGF-beta signaling pathway activator. In some embodiments, the TGF-beta signaling pathway activator is or comprises Activin A. In some embodiments, the PSCs are contacted with the TGF-beta signaling pathway activator (e.g. Activin A) at a concentration that is, is about, is at least, is at least about, is not more than, or is not more than about, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/mL, or any concentration within a range defined by any two of the aforementioned concentrations, for example, 15 to 45 ng/mL, 20 to 40 ng/mL, 15 to 30 ng/mL, or 30 to 45 ng/mL. In some embodiments, the PSCs are contacted with the TGF-beta signaling pathway activator (e.g. Activin A) at a concentration that is, is about, is at least, is at least about, is not more than, or is not more than about, 30 ng/mL.

In some embodiments, the PSCs are contacted with a Wnt signaling pathway activator. In some embodiments, the Wnt signaling pathway activator is or comprises CHIR99021. In some embodiments, the PSCs are contacted with the Wnt signaling pathway activator (e.g. CHIR99021) at a concentration that is, is about, is at least, is at least about, is not more than, or is not more than about, 1, 2, 3, 4, 5, 5.1, 5.6, 5.7, 5.8, 5.9, 6, 6.1, 6.2, 6.3, 6.4, 6.5, 6.6, 6.7, 6.8, 6.9, 7, 8, 9, or 10 μM, or any concentration within a range defined by any two of the aforementioned concentrations. In some embodiments, the PSCs are contacted with the Wnt signaling pathway activator (e.g. CHIR99021) at a concentration that is, is about, is at least, is at least about, is not more than, or is not more than about, 6 μM.

In some embodiments, the PSCs are contacted with an FGF signaling pathway activator. In some embodiments, the FGF signaling pathway activator is or comprises FGF2. In some embodiments, the PSCs are contacted with the FGF signaling pathway activator (e.g. FGF2) at a concentration that is, is about, is at least, is at least about, is not more than, or is not more than about, 1, 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations. In sonic embodiments, the PSCs are contacted with the FGF signaling pathway activator (e.g. FGF2) at a concentration that is, is about, is at least, is at least about, is not more than, or is not more than about, 20 ng/mL.

In some embodiments, the PSCs are contacted with a BMP signaling pathway activator. In some embodiments, the BMP signaling pathway activator is or comprises BMP4. In some embodiments, the PSCs are contacted with the BMP signaling pathway activator (e.g. BMP4) at a concentration that is, is about, is at least, is at least about, is not more than, or is not more than about, 20, 21, 22, 23, 24, 25, 26, 27, 28, 29, 30, 31, 32, 33, 34, 35, 36, 37, 38, 39, 40, 41, 42, 43, 44, 45.46, 47. 48, 49, 50, 51, 52, 53, 54, 55, 56, 57, 58, 59, or 60 ng/mL, or any concentration within a range defined by any two of the aforementioned concentrations. In some embodiments, the PSCs are contacted with the BMP signaling pathway activator (e.g. BMP4) at a concentration that is, is about, is at least, is at least about, is not more than, or is not more than about, 40 ng/mL.

In some embodiments, the PSCs are contacted with a PI3K signaling pathway inhibitor. In some embodiments, the PI3K signaling pathway inhibitor is or comprises PIK90. In some embodiments, the PSCs are contacted with the PI3K signaling pathway inhibitor (e.g. PIK90) at a concentration that is, is about, is at least, is at least about, is not more than, or is not more than about, 50, 60, 70, 80, 90, 100, 110, 120, 130, 140, or 150 nM, or any concentration within a range defined by any two of the aforementioned concentrations. In some embodiments, the PSCs are contacted with the PI3K signaling pathway inhibitor (e.g. PIK90) at a concentration that is, is about, is at least, is at least about, is not more than, or is not more than about, 100 nM.

In some embodiments, the PSCs are contacted with the TGF-beta signaling pathway activator, the Wnt signaling pathway activator, the FGF signaling pathway activator, the BMP signaling pathway activator, and the PI3K signaling pathway inhibitor for a time sufficient to differentiate the PSCs to middle primitive streak cells. In some embodiments, the PSCs are contacted for an amount of time that is, is about, is at least, is at least about, is not more than, or is not more than about, 12, 13, 14, 15, 16, 17, 18, 19, 20, 21, 22, 23, 24, 25, 26, 27, 28, 29, 30, 31, 32, 33, 34, 35, or 36 hours, or any amount of time within a range defined by any two of the aforementioned times. In some embodiments, the PSCs are contacted for an amount of time that is, is about, is at least, is at least about, is not more than, or is not more than about. 24 hours.

In some embodiments, any methods disclosed here or otherwise known in the art to differentiate middle primitive streak cells to lateral plate mesoderm cells are applicable. In some embodiments, the middle primitive streak cells have been differentiated from pluripotent stem cells. In some embodiments, the middle primitive streak cells are contacted with a TGF-beta signaling pathway inhibitor, a Wnt signaling pathway inhibitor, or a BMP signaling pathway activator, or any combination thereof, to differentiate the middle primitive streak cells to lateral plate mesoderm cells. In some embodiments, the TGF-beta signaling pathway inhibitor is selected from the group consisting of A8301, RepSox, LY365947, and SB431542. In some embodiments, the Wnt signaling pathway inhibitor is selected from the group consisting of C59, PNU 74654, KY-02111, PRI-724, FH-535, DIF-1, and XAV939. In some embodiments, the BMP signaling pathway activator is selected from the group consisting of BMP1, BMP2, BMP3, BMP4, BMP5, BMP6, BMP7, BMP8a, BMP8b, BMP10, BMP11, BMP15, IDE1, and IDE2. In some embodiments, the middle primitive streak cells are contacted with A8301, C59, BMP4, or any combination thereof, including all three, to differentiate the middle primitive streak cells to lateral plate mesoderm cells.

In some embodiments, the middle primitive streak cells are contacted with a TGF-beta signaling pathway inhibitor. In some embodiments, the TGF-beta signaling pathway inhibitor is or comprises A8301. In some embodiments, the middle primitive streak cells are contacted with the TGF-beta signaling pathway inhibitor (e.g. A8301) at a concentration that is, is about, is at least, is at least about, is not more than, or is not more than about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned times. In some embodiments, the middle primitive streak cells are contacted with the TGF-beta signaling pathway inhibitor (e.g. A8301) at a concentration that is, is about, is at least, is at least about, is not more than, or is not more than about, 1 μM.

In some embodiments, the middle primitive streak cells are contacted with a Wnt signaling pathway inhibitor. In some embodiments, the Wnt signaling pathway inhibitor is or comprises C59. In some embodiments, the middle primitive streak cells are contacted with the Wnt signaling pathway inhibitor (e.g. C59) at a concentration that is, is about, is at least, is at least about, is not more than, or is not more than about, 0.1, 0.2, 0.3, 0.4, 0.5, 0.6, 0.7, 0.8, 0.9, 1, 1.1, 1.2, 1.3, 1.4, 1.5, 1,6, 1.7, 1.8, 1.9, or 2 μM or any concentration within a range defined by any two of the aforementioned times. In some embodiments, the middle primitive streak cells are contacted with the Wnt signaling pathway inhibitor (e.g. C59) at a concentration that is, is about, is at least, is at leak about, is not more than, or is not more than about, 1 μM.

In some embodiments, the middle primitive streak cells are contacted with a BMP signaling pathway activator. In some embodiments, the BMP signaling pathway activator is or comprises BMP4. In some embodiments, the middle primitive streak cells are contacted with the BMP signaling pathway activator (e.g. BMP4) at a concentration that is, is about, is at least, is at least about, is not more than, or is not more than about, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/mL, or any concentration within a range defined by any two of the aforementioned concentrations. In some embodiments, the middle primitive streak cells are contacted with the BMP signaling pathway activator (e.g. BMP4) at a concentration that is, is about, is at least, is at least about, is not more than, or is not more than about, 30 ng/mL.

In some embodiments, the middle primitive streak cells are contacted with the TGF-beta signaling pathway inhibitor, the Wnt signaling pathway inhibitor, and the BMP signaling pathway activator for a time sufficient to differentiate the middle primitive streak cells to lateral plate mesoderm cells. In some embodiments, the middle primitive streak cells are contacted for an amount of time that is, is about, is at least, is at least about, is not more than, or is not more than about, 12, 13, 14, 15, 16, 17, 18, 19, 20, 21, 22, 23, 24, 25, 26, 27, 28, 29, 30, 31, 32, 33, 34, 35, or 36 hours, or any amount of time within a range defined by any two of the aforementioned times. In some embodiments, the middle primitive streak cells are contacted for an amount of time that is, is about, is at least, is at least about, is not more than, or is not more than about, 24 hours.

In some embodiments, lateral plate mesoderm cells are produced from pluripotent stem cells according to methods found in Loh et al. “Mapping the Pairwise Choices Leading from Pluripotency to Human Bone, Heart, and Other Mesoderm Cell Types” Cell. (2016) 166(2):451467, hereby expressly incorporated by reference for the purpose of differentiating lateral plate mesoderm cells and in its entirety.

### Differentiation to Splanchnic Mesoderm

Disclosed herein are methods of producing splanchnic mesoderm cells from lateral plate mesoderm cells. In some embodiments, the lateral plate mesoderm cells are produced according to any one of the methods disclosed herein or otherwise known in the art. The methods of producing splanchnic mesoderm cells comprise contacting lateral plate mesoderm cells with a TGF-beta signaling pathway inhibitor, a Wnt signaling pathway inhibitor, a BMP signaling pathway activator, an FGF signaling pathway activator, or a retinoic acid (RA) signaling pathway activator, or any combination thereof, including at least one of each. In some embodiments, the lateral plate mesoderm cells are contacted with a TGF-beta signaling pathway inhibitor, a Wnt signaling pathway inhibitor, a BMP signaling pathway activator, an FGF signaling pathway activator, and a RA signaling pathway activator. In some embodiments, the TGF-beta signaling pathway inhibitor is selected from the group consisting of A8301, RepSox, LY365947, and SB431542. In some embodiments, the Wnt signaling pathway inhibitor is selected from the group consisting of C59, PNU 74654, KY-02111, PRI-724, FH-535 DIF-1, and XAV939. In some embodiments, the BMP signaling pathway activator is selected from the group consisting of BMP1, BMP2, BMP3, BMP4, BMP5, BMP6, BMP7, BMP8a, BMP8b, BMP10, BMP11, BMP15, IDE1, and IDE2. In some embodiments, the FGF signaling pathway activator is selected from the group consisting of FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, and FGF23. In some embodiments, the RA signaling pathway activator is selected from the group consisting of retinoic acid, all-trans retinoic acid, 9-cis retinoic acid, CD437, EC23, BS 493, TTNPB, and AM580. In some embodiments, the TGF-beta signaling pathway inhibitor is A8301. In some embodiments, the Wnt signaling pathway inhibitor is C59. In some embodiments, the BMP signaling pathway activator is BMP4. In some embodiments, the FGF signaling pathway activator is FGF2. In some embodiments, the RA signaling pathway activator is RA. In some embodiments, the lateral plate mesoderm cells are contacted with A8301, BMP4, C59, FGF2, and RA. In some embodiments, the lateral plate mesoderm cells are contacted with the factors described herein, e.g. A8301, BMP4, C59, FGF2, and RA, for a period of time sufficient to differentiate the lateral plate mesoderm cells to splanchnic mesoderm. In some embodiments, the lateral plate mesoderm cells are contacted for a time that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours, or any time within a range defined by any two of the aforementioned times, for example, 1 to 72 hours, 12 to 36 hours, 1 to 48 hours, or 24 to 72 hours. In some embodiments, the lateral plate mesoderm cells are contacted for a time that is, is about, is at least, is at least about, is not more than, or is not more than about, 36, 37, 38, 39, 40, 41, 42, 43, 44, 45, 46, 47, 48, 49, 50, 51, 52, 53, 54, 55, 56, 57, 58, 59, or 60 hours, or any time within a range defined by any two of the aforementioned times, for example, 36 to 60 hours, 40 to 54 hours, 36 to 48 hours, or 48 to 60 hours. In some embodiments, the lateral plate mesoderm cells are contacted for a time that is, is about, is at least, is at least about, is not more than, or is not more than about, 48 hours.

In some embodiments, the lateral plate mesoderm cells are contacted with a TGF-beta signaling pathway inhibitor. In some embodiments, the TGF-beta signaling pathway inhibitor is or comprises A8301. In some embodiments, the lateral plate mesoderm cells are contacted with the TGF-beta signaling pathway inhibitor (e.g. A8301) at a concentration that is, is about, is at least, is at least about, is not more than, or is not more than about, 0.01, 0.1, 1, 2, 3, 4, 5, 6, 7, 8. 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20. In some embodiments, the lateral plate mesoderm cells are contacted with the TGF-beta signaling pathway inhibitor (e.g. A8301) at a concentration that is, is about, is at least, is at least about, is not more than, or is not more than about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, for example, 0.1 to 2 μM 0.5 to 1.5 μM, 0.1 to 1 μM, or 1 to 2 μM. In some embodiments, the lateral plate mesoderm cells are contacted with the TGF-beta signaling pathway inhibitor (e.g. A8301) at a concentration that is, is about, is at least, is at least about, is not more than, or is not more than about, 1 μM.

In some embodiments, the lateral plate mesoderm cells are contacted with a Wnt signaling pathway inhibitor. In some embodiments, the Wnt signaling pathway inhibitor is or comprises C59. In some embodiments, the lateral plate mesoderm cells are contacted with the Wnt signaling pathway inhibitor (e.g. C59) at a concentration that is, is about, is at least, is at least about, is not more than, or is not more than about, 0.01, 0,1, 1, 2, 3, 4, 5, 6, 7, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM. In some embodiments, the lateral plate mesoderm cells are contacted with the Wnt signaling pathway inhibitor e.g. C59) at a concentration that is, is about, is at least, is at least about, is not more than, or is not more than about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, for example, 0.1 to 2 μM, 0.5 to 1.5 μM, 0.1 to 1 μM, or 1 to 2 μM. In some embodiments, the lateral plate mesoderm cells are contacted with the Wnt signaling pathway inhibitor (e.g. C59 at a concentration that is, is about, is at least, is at least about, is not more than, or is not more than about, 1 μM.

In some embodiments, the lateral plate mesoderm cells are contacted with a BMP signaling pathway activator. in some embodiments, the BMP signaling pathway activator is or comprises BMP4. In some embodiments, the lateral plate mesoderm cells are contacted with the BMP signaling pathway activator (e.g. BMP4) at a concentration that is, is about, is at least, is at least about, is not more than, or is not more than about, 1, 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, for example, 1 to 100 ng/mL, 5 to 40 ng/mL, 10 to 80 ng/mL, 1 to 50 ng/mL, or 50 to 100 ng/mL. In some embodiments, the lateral plate mesoderm cells are contacted with the BMP signaling pathway activator (e.g. BMP4) at a concentration that is, is about, is at least, is at least about, is not more than, or is not more than about, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/mL, or any concentration within a range defined by any two of the aforementioned concentrations, for example, 15 to 45 ng/mL, 20 to 40 ng/mL, 15 to 30 ng/mL, or 30 to 45 ng/mL. In some embodiments, the lateral plate mesoderm cells are contacted with the BMP signaling pathway activator (e.g. BMP4) at a concentration that is, is about, is at least, is at least about, is not more than, or is not more than about, 30 ng/mL.

In some embodiments, the lateral plate mesoderm cells are contacted with an FGF signaling pathway activator. In some embodiments, the FGF signaling pathway activator is or comprises FGF2. In some embodiments, the lateral plate mesoderm cells are contacted with the FGF signaling pathway activator (e.g. FGF2) at a concentration that is, is about, is at leak, is at least about, is not more than, or is not more than about, 1, 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, for example, 1 to 100 ng/mL, 5 to 40 ng/mL 10 to 80 ng/mL, 1 to 50 ng/mL, or 50 to 100 ng/mL. In some embodiments, the lateral plate mesoderm cells are contacted with the FGF signaling pathway activator (e.g. FGF2) at a concentration that is, is about, is at least, is at least about, is not more than, or is not more than about, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 ng/mL, or any concentration within a range defined by any two of the aforementioned concentrations, for example, 5 to 35 ng/mL, 10 to 30 ng/mL, 5 to 20 ng/mL, or 20 to 35 ng/mL. In some embodiments, the lateral plate mesoderm cells are contacted with the FGF signaling pathway activator (e.g. FGF2) at a concentration that is, is about, is at least, is at least about, is not more than, or is not more than about, 20 ng/mL.

In some embodiments, the lateral plate mesoderm cells are contacted with a retinoic acid signaling pathway activator. In some embodiments, the retinoic acid signaling pathway activator is or comprises RA. In some embodiments, the lateral plate mesoderm cells are contacted with the retinoic acid signaling pathway activator (e.g. RA) at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM In some embodiments, the lateral plate mesoderm cells are contacted with the RA signaling pathway activator (e.g. RA) at a concentration that is, is about, is at least, is at least about, is not more than, or is not more than about, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.9, or 3 μM, or any concentration within a range defined by any two of the aforementioned concentrations, for example, 1 to 3 μM, 1.5 to 2.5 μM, 1 to 2 μM, or 2 to 3 μM. In some embodiments, the lateral plate mesoderm cells are contacted with the RA signaling pathway activator (e,g. RA) at a concentration that is, is about, is at least, is at least about, is not more than, or is not more than about, 2 μM.

In some embodiments, lateral plate mesoderm cells are contacted with a TGF-beta signaling pathway inhibitor at a concentration of 0.01-20 μM, a Wnt signaling pathway inhibitor at a concentration of 0.01-20, a BMP signaling pathway activator at a concentration of 1-100 ng/mL, an FGF signaling pathway activator at a concentration of 1-100 ng/mL, and a RA signaling pathway activator at a concentration of 0.01-20 μM. In some embodiments, lateral plate tnesoderm cells are contacted with a TGF-beta signaling pathway inhibitor at a concentration of 0.1-2 μM, a Wnt signaling pathway inhibitor at a concentration of 0.1-2 μM, a BMP signaling pathway activator at a concentration of 15-45 ng/mL, an FGF signaling pathway activator at a concentration of 5-35 ng/mL, and a RA signaling pathway activator at a concentration of 1-3 μM. In some embodiments, lateral plate mesoderm cells are contacted with A8301 at a concentration of 0.01-20 μM, C59 at a concentration of 0.01-20, BMP4 at a concentration of 1-100 ng/mL, FGF2 at a concentration of 1-100 ng/mL, and RA at a concentration of 0.01-20 μM. In some embodiments, lateral plate mesoderm cells are contacted with A8301 at a concentration of 0.1-2 μM, C59 at a concentration of 0.1-2 μM, BMP4 at a concentration of 15-45 ng/mL, FGF2 at a concentration of 5-35 ng/mL, and RA at a concentration of 1-3 μM. In some embodiments, lateral plate mesoderm cells are contacted with A8301 at a concentration of 1 μM, C59 at a concentration of 1 μM, BMP4 at a concentration of 30 ng/mL, FG-F2 at a concentration of 20 ng/mL, and RA at a concentration of 2 μM.

In some embodiments, the splanchnic mesoderm cells produced according to any of the methods herein exhibit increased expression of FOXF1, HOXA1, HOXA5, or WNT2, or any combination thereof, relative to cardiac mesoderm cells. In some embodiments, the splanchnic mesoderm cells exhibit decreased expression of NKX2-5, ISL1, or TBX2, or any combination thereof, relative to cardiac mesoderm cells. In some embodiments, the splanchnic mesoderm cells exhibit decreased expression of PAX3, or PRRX1, or both, relative to middle primitive streak cells. In some embodiments, the splanchnic mesoderm cells exhibit decreased expression of CD31 relative to cardiac mesoderm cells.

In any of the embodiments provided herein, the splanchnic mesoderm cells are mammalian cells. In some embodiments, the splanchnic mesoderm cells are human splanchnic mesoderm cells. In some embodiments, the splanchnic mesoderm cells are derived from a subject. In some embodiments, the subject is a human. In some embodiments, the subject has a disease or is at risk of contracting a disease. In some embodiments, the splanchnic mesoderm cells are derived from PSCs derived from the subject.

### Differentiation to Splanchnic Mesoderm Cell Types

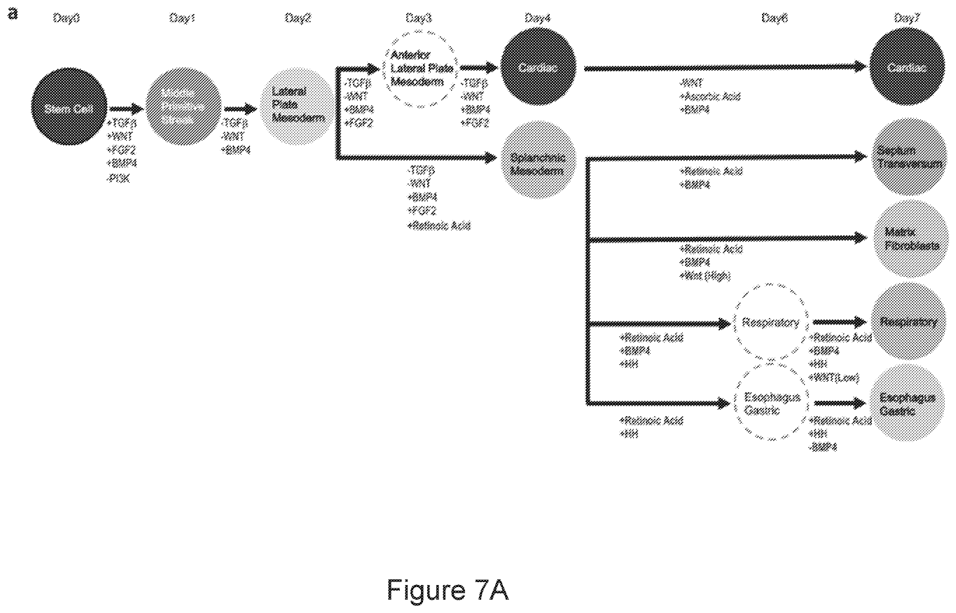

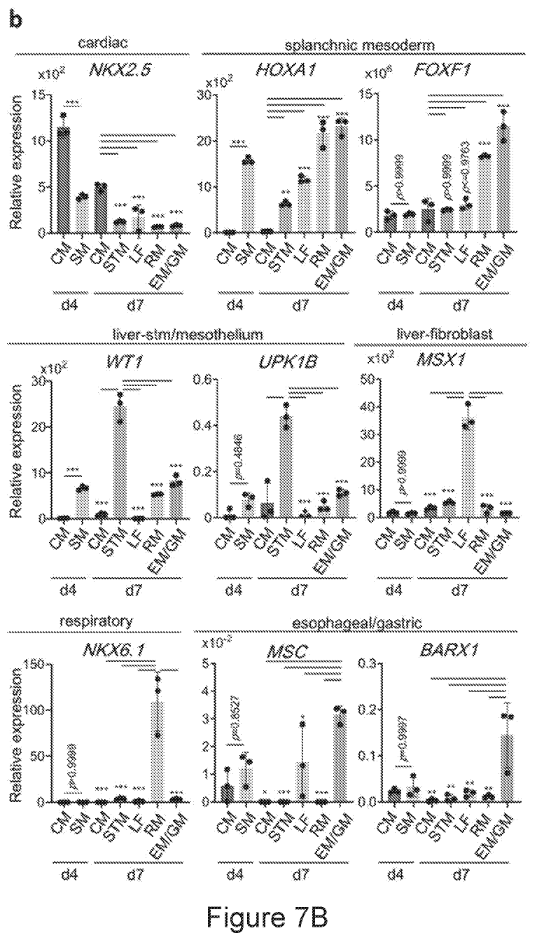

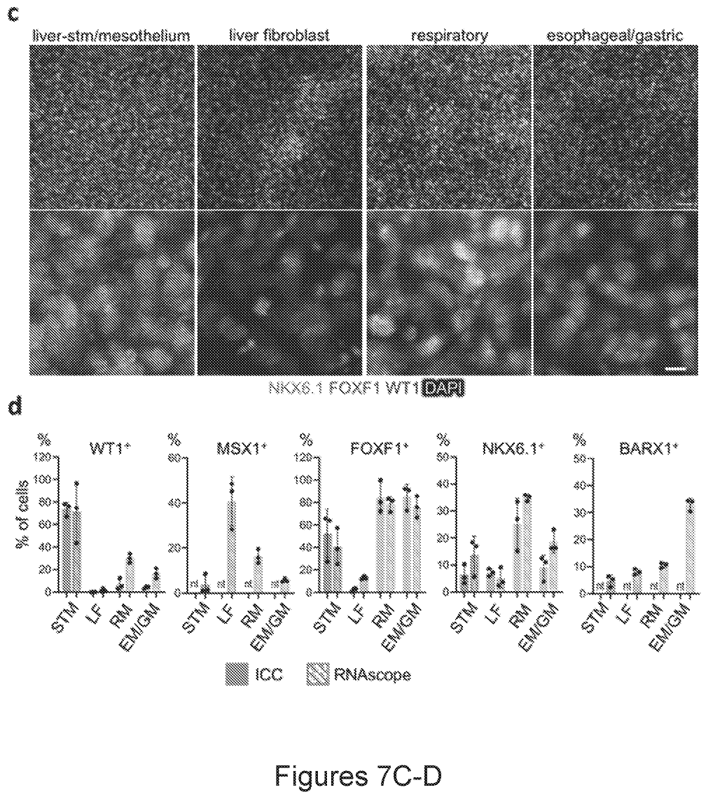

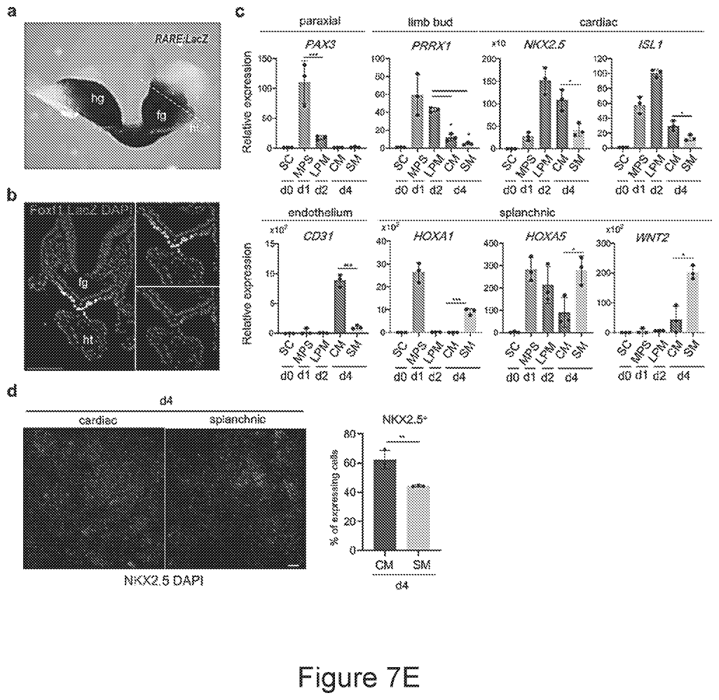

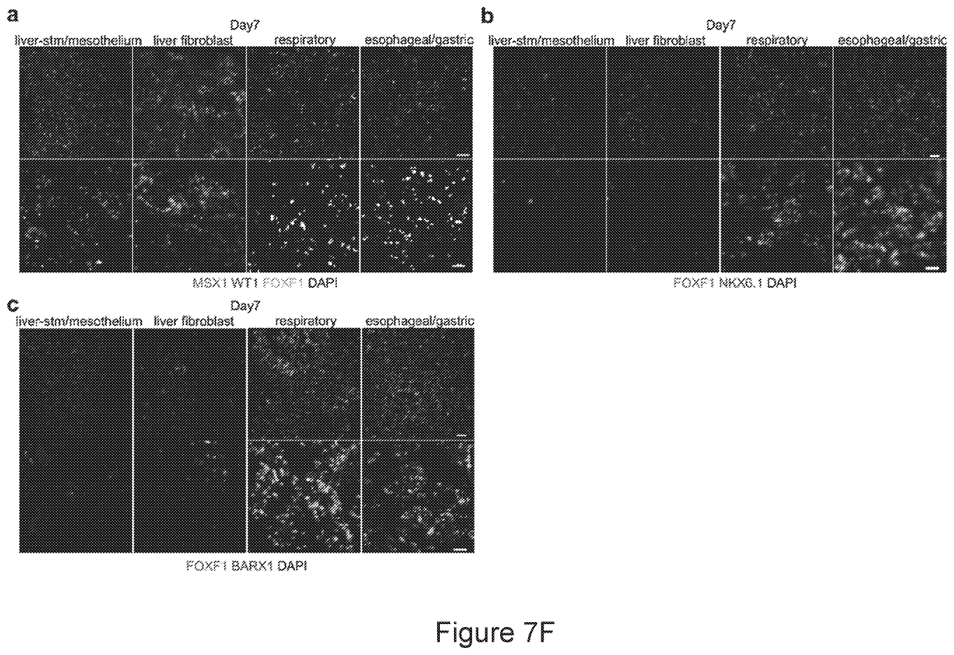

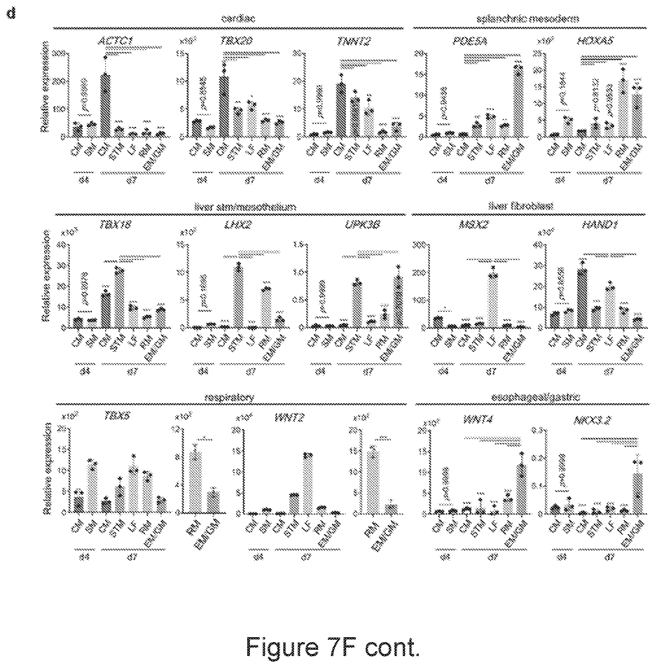

As disclosed herein, the splanchnic mesoderm cells produced by any of the methods herein can be further differentiated into splanchnic mesoderm subtypes. In some embodiments, the splanchnic mesoderm subtypes comprise septum transversum cells, fibroblasts, respiratory rnesenchyrne cells, or esophageal/gastric rnesenchyrne cells, or any combination thereof. In some embodiments, the septum transversum cells comprise liver septum transversum cells. In some embodiments, the fibroblasts comprise liver fibroblasts. Embodiments of the differentiation of splanchnic mesoderm cells into splanchnic mesoderm subtypes are disclosed in FIG. 7A.

Production of Septum Transversum Cells

In some embodiments are methods comprising contacting splanchnic mesoderm cells with a retinoic acid signaling pathway activator, or a BMP signaling pathway activator, or both. In some embodiments, the splanchnic mesoderm cells are the splanchnic mesoderm cells produced by any of the methods described herein. In some embodiments, this contacting differentiates the splanchnic mesoderm cells to septum transversum cells. In some embodiments, the splanchnic mesoderm cells are contacted with a retinoic acid signaling pathway activator and a BMP signaling pathway activator. In some embodiments, the retinoic acid signaling activator is selected from the group consisting of retinoic acid, all-trans retinoic acid, 9-cis retinoic acid, CD437, EC23, BS 493, TTNPB, and AM580. In some embodiments, the BMP signaling pathway activator is selected from the group consisting of BMP1, BMP2, BMP3, BMP4, BMP5, BMP6, BMP7, BMP8a, BMP8b, BMP10, BMP11, BMP15, IDE1, and IDE2. In some embodiments, the retinoic acid signaling pathway activator is RA. In some embodiments, the BMP signaling pathway activator is BMP4. In some embodiments, the splanchnic mesoderm cells are contacted with RA, BMP4, or both.

In some embodiments, the splanchnic mesoderm cells are contacted with the retinoic acid signaling pathway activator (e.g. RA) at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 1 to 15 μM, or 10 to 20 μM, and the BMP signaling pathway activator (e.g. BMP4) at a concentration that is, is about, is at least, is at least about, is not more than, or is not more than about, 1, 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, for example, 1 to 100 ng/mL, 5 to 40 ng/mL, 10 to 80 ng/mL 1 to 50 ng/mL, or 50 to 100 ng/mL. In sonic embodiments, the splanchnic mesoderm cells are contacted with the retinoic acid signaling pathway activator (e.g. RA) at a concentration that is, is about, is at least, is at least about, is not more than, or is not more than about, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM, or any concentration within a range defined by any two of the aforementioned concentrations, and the BMP signaling pathway activator (e.g. BMP4) at a concentration that is, is about, is at least, is at least about, is not more than, or is not more than about, 10, 20, 30, 40, 50, 60, 70, or 80 ng/mL, or any concentration within a range defined by any two of the aforementioned concentrations. In some embodiments, the splanchnic mesoderm cells are contacted with the retinoic acid signaling pathway activator (e.g. RA) at a concentration that is, is about, is at least, is at least about, is not more than, or is not more than about, 1.8, 1.9, 2, 2.1, or 2.2 μM, or any concentration within a range defined by any two of the aforementioned concentrations, and the BMP signaling pathway activator (e.g. BMP4) at a concentration that is, is about, is at least, is at least about, is not more than, or is not more than about, 20, 30, 40, 50, or 60 ng/mL, or any concentration within a range defined by any two of the aforementioned concentrations. In some embodiments, the splanchnic mesoderm cells are contacted with the retinoic acid signaling pathway activator (e.g. RA) at a concentration that is, is about, is at least, is at least about, is not more than, or is not more than about, 2 μM, and the BMP signaling pathway activator (e.g. BMP4) at a concentration that is, is about, is at least, is at least about, is not more than, or is not more than about, 40 ng/mL.

In some embodiments, the splanchnic mesoderm cells are contacted with an RA signaling pathway activator at a concentration of 0.01-20 μM, and a BMP signaling pathway activator at a concentration of 1-100 ng/mt. In some embodiments, the splanchnic mesoderm cells are contacted with an RA signaling pathway activator at a concentration of 1-3 μM, and a BMP signaling pathway activator at a concentration of 10-80 ng/mL. In some embodiments, the splanchnic mesoderm cells are contacted with RA at a concentration of 0.01-20 μM, and BMP4 at a concentration of 1-100 ng/mL. In some embodiments, the splanchnic mesoderm cells are contacted with RA at a concentration of 1-3 μM, and BMP4 at a concentration of 10-80 ng/mL. In some embodiments, the splanchnic mesoderm cells are contacted with RA at a concentration of 2 μM, and BMP4 at a concentration of 40 ng/mL.

In some embodiments, the retinoic acid signaling pathway activator (e.g. RA), or BMP signaling pathway activator (e.g. BMP4), or both, are contacted in the concentrations described herein for a period of time sufficient to differentiate the splanchnic mesoderm cells to septum transversum cells. In some embodiments, the splanchnic mesoderm cells are contacted with the factors described herein, e.g. RA and BMP4, for a period of time sufficient to differentiate the splanchnic mesoderm cells to septum transversum cells. In some embodiments, the contacting is for a period of time that is, is about, is at least, is at least about, is not more than, or is not more than about, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, or 108 hours, or any period of time within a range defined by any two of the aforementioned times. In some embodiments, the contacting is for a period of time that is, is about, is at least, is at least about, is not more than, or is not more than about, 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times. In some embodiments, the contacting is for a period of time that is, is about, is at least, is at least about, is not more than, or is not more than about, 72 hours.

In some embodiments, the resulting septum transversum cells exhibit increased expression of WT1, TBX18, LHX2, UPK3B, or UPK1B, or any combination thereof, relative to cardiac mesoderm cells, splanchnic mesoderm cells, or fibroblasts, or any combination thereof. In some embodiments, the septum transversum cells exhibit decreased expression of MSX1, MSX2, or HAND1 or any combination thereof, relative to cardiac mesoderm cells or fibroblasts, or both. In some embodiments, the septum transversum cells exhibit decreased expression of HOXA1, or TBX5, or both, relative to splanchnic mesoderm cells. In some embodiments, the septum transversum cells exhibit decreased expression of NKX6.1 or HOXA5, or both, relative to respiratory mesenchyme cells. In some embodiments, the septum transversum cells exhibit decreased expression of NKX3.2, MSC, BARX1, WNT4, or HOXA5, or any combination thereof, relative to esophageal/gastric mesenchyme cells. In sonic embodiments, the septum transversum cells account for a percentage of total cells differentiated from the splanchnic mesoderm cells that is, is about, is at leak, is at least about, is not more than, or is not more than about, 60%, 65%, 70%, 75%, 80%, 85%, 90%, or 100% of the total cells differentiated from the splanchnic mesoderm cells, or any percentage within a range defined by any two of the aforementioned percentages, for example, 60% to 100%, 70% to 90%, or 75% to 85%.

Production of Fibroblast Cells

In some embodiments are methods comprising contacting splanchnic mesoderm cells with a retinoic acid signaling pathway activator, a BMP signaling pathway activator, or a Wnt signaling pathway activator, or any combination thereof. In some embodiments, the splanchnic mesoderm cells are the splanchnic mesoderm cells produced by any of the methods described herein. In some embodiments, this contacting differentiates the splanchnic mesoderm cells to fibroblasts. In some embodiments, the splanchnic mesoderm cells are contacted with a retinoic acid signaling pathway activator, a BMP signaling pathway activator and a Wnt signaling pathway activator. In some embodiments, the retinoic acid signaling pathway activator is selected from the group consisting of retinoic acid, all-trans retinoic acid, 9-cis retinoic acid, CD437 EC23, BS 493, TTNPB, and AM580. In some embodiments the BMP signaling pathway activator is selected from the group consisting of BMP1, BMP2, BMP3, BMP4, BMP5, BMP6, BMP7, BMP8a, BMP8b, BMP10, BMP11, BMP15, IDE1, and IDE2. In some embodiments, the Wnt signaling pathway activator is selected from the group consisting of Wnt1, Wnt2 Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, and TWS119. In some embodiments, the retinoic acid signaling pathway activator is RA. In some embodiments, the BMP signaling pathway activator is BMP4. In some embodiments, the Wnt signaling pathway activator is CHIR99021. In some embodiments, the splanchnic mesoderm cells are contacted with RA, BMP4, CHIR99021, or any combination thereof, including all three.

In some embodiments, the splanchnic mesoderm cells are contacted with the RA signaling pathway activator (e.g. RA) at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 1.5 μM, or 10 to 20 μM, the BMP signaling pathway activator (e.g. BMP4) at a concentration that is, is about, is at least, is at leak about, is not more than, or is not more than about, 1, 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, for example, 1 to 100 ng/mL, 5 to 40 ng/mL, 10 to 80 ng/mL, 1 to 50 ng/mL, or 50 to 100 ng/mL, and the Wnt signaling pathway activator (e.g. CHIR99021) at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM., or any concentration within a range defined by any two of the aforementioned concentrations, for example, 0.01 to 20 μM, 0.01 to 10 μM, 1 to 15 μM, or 10 to 20 μM. In some embodiments, the splanchnic mesoderm cells are contacted with the RA signaling pathway activator (e.g. RA) at a concentration that is, is about, is at least, is at least about, is not more than, or is not more than about, 1, 1.1, 1.2. 1.3, 1.4, 1.5, 1.6, 1.7, 1.8, 1.9, 2, 2.1, 2.2, 2.3, 2.4, 2.5, 2.6, 2.7, 2.8, 2.9, or 3 μM, or any concentration within a range defined by any two of the aforementioned concentrations, the BMP signaling pathway activator (e.g. BMP4) at a concentration that is, is about, is at least, is at least about, is not more than, or is not more than about, 10, 20, 30, 40, 50, 60, 70, or 80 ng/mL, or any concentration within a range defined by any two of the aforementioned concentrations, and the Wnt signaling pathway activator (e.g. CHIR99021) at a concentration that is, is about, is at least, is at least about, is not more than, or is not more than about, 1, 2, 3, 4, 5, 5.1, 5.6, 5.7, 5.8, 5.9, 6, 6.1, 6.2, 6.3, 6.4, 6.5, 6.6, 6.7, 6.8, 6.9, 7, 8, 9, or 10 μM, or any concentration within a range defined by any two of the aforementioned concentrations. In some embodiments, the splanchnic mesoderm cells are contacted with the RA signaling pathway activator (e.g. RA) at a concentration that is, is about, is at least, is at least about, is not more than, or is not more than about, 2 μM, the BMP signaling pathway activator (e.g. BMP4) at a concentration that is, is about, is at least, is at least about, is not more than, or is not more than about, 40 ng/mL, and the Wnt signaling pathway activator (e.g. CHIR99021) at a concentration that is, is about, is at least, is at least about, is not more than, or is not more than about, 6 μM.

In some embodiments, the splanchnic mesoderm cells are contacted with an RA signaling pathway activator at a concentration of 001-20 μM, a BMP signaling pathway activator at a concentration of 1-100 ng/mL, and a Wnt signaling pathway activator at a concentration of 0.01-20 μM. In some embodiments, the splanchnic mesoderm cells are contacted with an RA signaling pathway activator at a concentration of 1-3 μM, a BMP signaling pathway activator at a concentration of 10-80 ng/mL, and a Wnt signaling pathway activator at a concentration of 5-7 μM In some embodiments, the splanchnic mesoderm cells are contacted with RA at a concentration of 0.01-20 μM, BMP4 at a concentration of 1-100 ng/mL, and CHIR99021 at a concentration of 0.01-20 μM. In some embodiments, the splanchnic mesoderm cells are contacted with RA at a concentration of 1-3 μM, BMP4 at a concentration of 10-80 ng/mL, and CHIR99021 at a concentration of 5-7 μM. In some embodiments, the splanchnic mesoderm cells are contacted with RA at a concentration of 2 μM, BMP4 at a concentration of 40 ng/mL, and CHIR99021 at a concentration of 6 μM.

In some embodiments, the RA signaling pathway activator (e.g. RA), the BMP signaling pathway activator (e.g. BMP4), and the Wnt signaling pathway activator (e.g. CHIR99021) are contacted in the concentrations described herein for a period of time sufficient to differentiate the splanchnic mesoderm cells to fibroblasts. In some embodiments, the splanchnic mesoderm cells are contacted with the factors described herein, e.g. RA, BMP4, and CHIR99021, for a period of time sufficient to differentiate the splanchnic mesoderm cells to fibroblasts. In some embodiments, the contacting is for a period of time that is, is about, is at least, is at least about, is not more than, or is not more than about, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, or 108 hours, or any period of time within a range defined by any two of the aforementioned times. In some embodiments, the contacting is for a period of time that is, is about, is at least, is at least about, is not more than, or is not more than about, 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times. In some embodiments, the contacting is for a period of time that is, is about, is at least, is at least about, is not more than, or is not more than about, 72 hours.

In some embodiments, the fibroblasts exhibit increased expression of MSX1, MSX2, or HAND1, or any combination thereof, relative to splanchnic mesoderm cells, or septum transversum cells, or both. In some embodiments, the fibroblasts exhibit decreased expression of WT1, TBX18, LHX2, or UPK1B, or any combination thereof, relative to septum transversum cells. In sonic embodiments, the fibroblasts exhibit decreased expression of NKX6.1, HOXA5, or LHX2, or any combination thereof, relative to respiratory mesenchyme cells. In some embodiments, the fibroblasts exhibit decreased expression of NKX3.2, MSC, BARX1, WNT4, or HOXA5, or any combination thereof, relative to esophageal/gastric mesenchyme cells.

Production of Respiratory Mesenchyme Cells

In some embodiments are methods comprising contacting splanchnic mesoderm cells with a RA signaling pathway activator, a BMP signaling pathway activator, a HFI signaling pathway activator, or a Wnt signaling pathway activator, or any combination thereof. In sonic embodiments, the splanchnic mesoderm cells are the splanchnic mesoderm cells produced by any of the methods described herein. In some embodiments, this contacting differentiates the splanchnic mesoderm cells to respiratory mesenchyme cells. In some embodiments, the splanchnic mesoderm cells are contacted with a RA signaling pathway activator, a BMP signaling pathway activator, a HH signaling pathway activator, and a Wnt signaling pathway activator. In some embodiments, the methods may further comprise contacting the splanchnic mesoderm cells with a retinoic acid signaling pathway activator, a BMP signaling pathway activator, and a HH signaling pathway activator prior to contacting the splanchnic mesoderm cells with the RA signaling pathway activator, the BMP signaling pathway activator, the HH signaling pathway activator, and the Wnt signaling pathway activator. In some embodiments, this two-step process enhances the differentiation of the splanchnic mesoderm cells to respiratory mesenchyme cells.

In some embodiments, the RA signaling pathway activator is selected from the group consisting of retinoic acid, all-trans retinoic acid, 9-cis retinoic acid, CD437, EC23, BS 493, TTNPB, and AM580. In some embodiments, the BMP signaling pathway activator is selected from the group consisting of BMP1, BMP2, BMP3, BMP4, BMP5, BMP6, BMP7, BMP8a, BMP8b, BMP10, BMP11, BMP15, IDE1 and IDE2. In some embodiments, the HH signaling pathway activator is selected from the group consisting of SHH, IHH, DHH, PMA, GSA 10, and SAG. In some embodiments, the Wnt signaling pathway activator is selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014 AZD2858, BIO, AR-A014418, SB 216763, SB 415286, aloisine, indirubin, alsterpaullone, kenpaullone, lithium chloride, TDZD 8, and TWS119. In some embodiments, the RA signaling pathway activator is RA. In some embodiments, the BMP signaling pathway activator is BMP4. In some embodiments, the signaling pathway activator is PMA. In sonic embodiments, the Wnt signaling pathway activator is CHIR99021. In some embodiments, the splanchnic mesoderm cells are contacted with RA, BMP4, PMA, CHIR99021, or any combination thereof, including all four.

In some embodiments, the splanchnic mesoderm cells are contacted with the RA signaling pathway activator (e.g. RA) at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM, the BMP signaling pathway activator (e.g. BMP4) at a concentration that that is, is about, is at least, is at least about, is not more than, or is not more than about, 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, for example, 1 to 100 ng/mL, 5 to 40 ng/mL, 10 to 80 ng/mL, 1 to 50 ng/mL, or 50 to 100 ng/mL, the HH signaling pathway activator (e.g. PMA) at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM, and optionally, the Wnt signaling pathway activator (e.g. CHIR99021) at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM.

In some embodiments, the splanchnic mesoderm cells are contacted with the RA signaling pathway activator (e.g. RA) at a concentration that is, is about, is at least, is at least about, is not more than, or is not more than about, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 or any concentration within a range defined by any two of the aforementioned concentrations, the BMP signaling pathway activator (e.g. BMP4) at a concentration that is, is about, is at least, is at least about, is not more than, or is not more than about, 10, 20, 30, 40, 50, 60, 70, or 80 ng/mL, or any concentration within a range defined by any two of the aforementioned concentrations, the HH signaling pathway activator (e.g. PMA) at a concentration that is, is about, is at least, is at least about, is not more than, or is not more than about, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM, or any concentration within a range defined by any two of the aforementioned concentrations, and optionally the Wnt signaling pathway activator (e.g. CHIR99021) at a concentration that is, is about, is at least, is at least about, is not more than, or is not more than about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, for example, 0.1 to 2 μM, 0.5 to 1.5 μM, 0.1 to 1 μM, or 1 to 2 μM.

In some embodiments, the splanchnic mesoderm cells are contacted with the RA signaling pathway activator (e.g. RA), at a concentration that is is about, is at least, is at least about, is not more than, or is not more than about, 2 μM, the BMP signaling pathway activator (e.g. BMP4) at a concentration that is, is about is at least, is at least about, is not more than, or is not more than about, 40 ng/mL, the HH signaling pathway activator (e.g. PMA) at a concentration that is, is about, is at least, is at least about, is not more than, or is not more than about, 2 μM, and optionally the Wnt signaling pathway activator (e.g. CHIR99021), at a concentration that is, is about, is at least, is at least about, is not more than, or is not more than about, 1 μM.

In some embodiments, the splanchnic mesoderm cells are contacted with an RA signaling pathway activator at a concentration of 0.01-20 μM, a BMP signaling pathway activator at a concentration of 1-100 ng/mL, a HH signaling pathway activator at a concentration of 0.01-20 μM, and optionally a Wnt signaling pathway activator at a concentration of 0.01-20 μM. In some embodiments, the splanchnic mesoderm cells are contacted with an RA signaling pathway activator at a concentration of 1-3 μM, a BMP signaling pathway activator at a concentration of 10-80 ng/mL, a HH signaling pathway activator at a concentration of 1-3 μM, and optionally a Wnt signaling pathway activator at a concentration of 0.1-2 μM. In some embodiments, the splanchnic mesoderm cells are contacted with RA at a concentration of 0.01-20 μM, BMP4 at a concentration of 1-100 ng/mL, PMA at a concentration of 0.01-20 μM, and optionally CHIR99021 at a concentration of 0.01-20 μM. In some embodiments, the splanchnic mesoderm cells are contacted with RA at a concentration of 1-3 μM, BMP4 at a concentration of 10-80 ng/mL, PMA at a concentration of 1-3 μM, and optionally CHIR99021 at a concentration of 0.1-2 μM. In some embodiments, the splanchnic mesoderm cells are contacted with RA at a concentration of 2 μM, BMP4 at a concentration of 40 ng/mL, PMA at a concentration of 2 μM, and optionally CHIR99021 at a concentration of 1 μM.

In some embodiments, the splanchnic mesoderm cells are differentiated to respiratory mesenchyme cells in a one-step process. In these embodiments, the methods comprise contacting splanchnic mesoderm cells with a RA signaling pathway activator (e.g. RA), a BMP signaling pathway activator (e.g. BMP4), a HH signaling pathway activator (e.g. PMA), and a Wnt signaling pathway activator (e.g. CHIR99021). In some embodiments, the RA signaling pathway activator the BMP signaling pathway activator, and the Wnt signaling pathway activator of the one-step process are contacted in the concentrations described herein for a period of time sufficient to differentiate the splanchnic mesoderm cells to respiratory mesenchyme cells. In some embodiments, the splanchnic mesoderm cells are contacted with the factors described herein, e.g. RA, BMP4, PMA, and CHIR99021, for a period of time sufficient to differentiate the splanchnic mesoderm cells to respiratory mesenchyme cells. In some embodiments, the contacting is for a period of time that is, is about, is at least, is at least about, is not more than, or is not more than about, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, or 108 hours, or any period of time within a range defined by any two of the aforementioned times. In some embodiments, the contacting is for a period of time that is, is about, is at least, is at least about, is not more than, or is not more than about, 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times. In some embodiments, the contacting is for a period of time that is, is about, is at least, is at least about, is not more than, or is not more than about, 72 hours.

In some embodiments, the splanchnic mesoderm cells are differentiated to respiratory mesenchyme cells in a two-step process. In these embodiments, the methods comprise a first step of contacting the splanchnic mesoderm cells with a :RA signaling pathway activator, a BMP signaling pathway activator, and a HH signaling pathway activator prior to a second step of contacting the splanchnic mesoderm cells with a RA signaling pathway activator, a BMP signaling pathway activator, a HH signaling pathway activator, and a Wnt signaling pathway activator (e.g. CHIR99021). In some embodiments the RA signaling pathway activator (e.g. RA), the BMP signaling pathway activator (e.g. BMP4), and the MI signaling pathway activator (e.g. PMA) of the first step and the second step are the same. In some embodiments, the RA signaling pathway activator, the BMP signaling pathway activator, and the HH signaling pathway activator of the first step and the second step are different. In some embodiments, the RA signaling pathway activator, the BMP signaling pathway activator, and the HH signaling pathway activator of the first step of the two-step process and the RA signaling pathway activator, the BMP signaling pathway activator, the HH signaling pathway activator, and the Wnt signaling pathway activator of the second step of the two-step process are contacted in the concentrations described herein for a period of time sufficient to differentiate the splanchnic mesoderm cells to respiratory mesenchyme cells. In some embodiments, the splanchnic mesoderm cells are contacted with the factors described herein, e.g. RA, BMP4, PMA, and CHIR99021, for a period of time sufficient to differentiate the splanchnic mesoderm cells to respiratory mesenchyme cells. In some embodiments, the RA signaling pathway activator (e.g. RA), the BMP signaling pathway activator (e.g. BMP4), and the HH signaling pathway activator (e.g. PMA) of the first step are contacted for a period of time that is, is about, is at least, is at least about, is not more than, or is not more than about, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 ,43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times. In some embodiments, the RA signaling pathway activator (e.g, RA), the BMP signaling pathway activator (e.g. BMP4), and the HH signaling pathway activator (e.g. PMA) of the first step are contacted for a period of time that is, is about, is at least, is at least about, is not more than, or is not more than about, 36, 37, 38, 39, 40, 41, 42, 43, 44, 45, 46, 47, 48, 49, 50, 51, 52, 53, 54, 55, 56, 57, 58, 59, or 60 hours, or any period of time within a range defined by any two of the aforementioned times. In some embodiments, the RA signaling pathway activator (e.g. RA), the BMP signaling pathway activator (e.g. BMP4), and the HH signaling pathway activator (e.g. PMA) of the first step are contacted for a period of time that is, is about, is at least, is at least about, is not more than, or is not more than about, 48 hours. In some embodiments, the RA signaling pathway activator (e.g. RA), the BMP signaling pathway activator (e.g. BMP4), the HH signaling pathway activator (e.g. PMA), and the Wnt signaling pathway activator (e.g. CHIR99021) of the second step are contacted for a period of time that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32,33 34, 35, 36, 37, 38, 39, 40, 41, 42, 43, 44, 45, 46, 47, or 48 hours, or any period of time within a range defined by any two of the aforementioned times. In some embodiments, the RA signaling pathway activator (e.g. RA), the BMP signaling pathway activator (e.g. BMP4), the HH signaling pathway activator (e.g. PMA), and the Wnt signaling pathway activator (e.g. CHIR99021) of the second step are contacted for a period of tune that is, is about, is at least, is at least about, is not more than, or is not more than about, 12, 13, 14, 15, 16, 17, 18, 19, 20, 21, 22, 23, 24, 25, 26, 27, 28, 29, 30, 31, 32, 33, 34, 35, or 36 hours, or any period of time within a range defined by any two of the aforementioned times. In some embodiments, the RA signaling pathway activator (e.g. RA), the BMP signaling pathway activator (e.g. BMP4), the HH signaling pathway activator (e.g. PMA), and the Wnt signaling pathway activator (e.g. CHIR99021) of the second step are contacted for a period of time that is, is about, is at least, is at least about, is not more than, or is not more than about, 24 hours.

In some embodiments, the respiratory mesenchyme cells exhibit increased expression of NKX6-1, TBX5, HOXA1, HOXA5, FOXE1, LHX2, or WNT2, or any combination thereof, relative to cardiac endoderm cells, splanchnic mesoderm cells, or esophageal/gastric mesenchyme cells, or any combination thereof. In some embodiments, the respiratory mesenchyme cells exhibit decreased expression of WNT2, WT1, TBX18, LHX2, or UPK1B, or any combination thereof, relative to septum transversum cells. In some embodiments, the respiratory mesenchyme cells exhibit decreased expression of WNT2, MSX1, or MSX2, or any combination thereof, relative to fibroblast cells.

Production of Esophageal/Gastric Mesenchyme Cells

In some embodiments are methods comprising contacting splanchnic mesoderm cells with a RA signaling pathway activator, a HH signaling pathway activator, or a BMP signaling pathway inhibitor, or any combination thereof. In some embodiments, the splanchnic mesoderm cells are the splanchnic mesoderm cells produced by any of the methods described herein. In some embodiments, this contacting differentiates the splanchnic mesoderm cells to esophageal/ga.stric mesenchyme cells. In some embodiments, the splanchnic mesoderm cells are contacted with a RA signaling pathway activator, a HH signaling pathway activator, and a BMP signaling pathway inhibitor. In some embodiments, the methods may further comprise contacting the splanchnic mesoderm cells with a retinoic acid signaling pathway activator and a HH signaling pathway activator prior to contacting the splanchnic mesoderm cells with the retinoic acid signaling pathway activator, the HH signaling pathway activator, and the BMP signaling pathway activator. In some embodiments, this two-step process enhances the differentiation of the splanchnic mesoderm cells to esophageal/gastric mesenchyme cells.

In some embodiments, the RA signaling pathway activator is selected from the group consisting of retinoic acid, all-trans retinoic acid, 9-cis retinoic acid, CD437, EC23, BS 493, TTNPB, and AM580. In some embodiments, the HH signaling pathway activator is selected from the group consisting of SHH, IHH, DHH, PMA, GSA 10, and SAG. In some embodiments, the BMP signaling pathway inhibitor is selected from the group consisting of Noggin, RepSox, LY364947, LDN193189, and SB431542. In some embodiments, the RA signaling pathway activator is RA. In some embodiments, the HH signaling pathway activator is PMA. In some embodiments, the BMP signaling pathway inhibitor is Noggin. In some embodiments, the splanchnic mesoderm cells are contacted with RA, RMA, Noggin or any combination thereof, including all three.

In some embodiments, the splanchnic mesoderm cells are contacted with the RA signaling pathway activator (e.g. RA) at a concentration that is, is about, is at least, is at least about, is not more than, or is not more than about, 0.01, 0.1, 1, 2, 3, 4. 5, 6, 7, 8, 9, 10, 11, 12, 13, 14, 15. 16, 17, 18, 19, or 20 or any concentration within a range defined by any two of the aforementioned concentrations, for example, 0.01 to 20 μM, 0.01 to 10 μM, 1 to 15 μM, or 10 to 20 μM, the HH signaling pathway activator (e.g. PMA) at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM, and optionally the BMP signaling pathway inhibitor (e.g. Noggin) at a concentration that is, is about, is at least, is at least about, is not more than, or is not more than about, 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, or 250 ng/mL, or any concentration within a range defined by any two of the aforementioned concentrations, for example, 1 to 250 ng/mL, 5 to 150 ng/mL, 10 to 100 ng/mL, 1 to 150 ng/mL, or 50 to 250 ng/mL.

In some embodiments, the splanchnic mesoderm cells are contacted with the RA signaling pathway activator (e.g. RA) at a concentration that is, is about, is at least, is at least about, is not more than, or is not more than about, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM, or any concentration within a range defined by any two of the aforementioned concentrations, the HH signaling pathway activator (e.g. PMA) at a concentration that is, is about, is at least, is at least about, is not more than, or is not more than about, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM, or any concentration within a range defined by any two of the aforementioned concentrations, and optionally the BMP signaling pathway inhibitor (e.g. Noggin) at a concentration that is, is about, is at least, is at least about, is not more than, or is not more than about, 50, 60, 70, 80, 90, 100, 110, 120, 130, 140, or 150 ng/mL, or any concentration within a range defined by any two of the aforementioned concentrations.

In some embodiments, the splanchnic mesoderm cells are contacted with the RA signaling pathway activator (e.g. RA) at a concentration that is, is about, is at least, is at least about, is not more than, or is not more than about, 2 μM, the HH signaling pathway activator (e.g. PMA) at a concentration that is, is about, is at least, is at least about, is not more than, or is not more than about, 2 μM, and optionally the BMP signaling pathway inhibitor (e.g. Noggin) at a concentration that is, is about, is at least, is at least about, is not more than, or is not more than about, 100 ng/mL.

In some embodiments, the splanchnic mesoderm cells are contacted with a RA signaling pathway activator at a concentration of 0.01-20 μM, a HH signaling pathway activator at a concentration of 0.01-20 μM, and optionally a BMP signaling pathway inhibitor at a concentration of 1-250 ng/mL. In some embodiments, the splanchnic mesoderm cells are contacted with a RA signaling pathway activator at a concentration of 1-3 μM, a HH signaling pathway activator at a concentration of 1-3 μM, and optionally a BMP signaling pathway inhibitor at a concentration of 50-150 ng/mL. In some embodiments, the splanchnic mesoderm cells are contacted with RA at a concentration of 0.01-20 μM, PMA at a concentration of 0.01-20 μM, and optionally Noggin at a concentration of 1-250 ng/mL. In some embodiments, the splanchnic mesoderm cells are contacted with RA at a concentration of 1-3 μM, PMA at a concentration of 1-3 μM, and optionally Noggin at a concentration of 50-150 ng/mL. In some embodiments, the splanchnic mesoderm cells are contacted with RA at a concentration of 2 μM, PMA at a concentration of 2 μM, and optionally Noggin at a concentration of 100 ng/mL.

In some embodiments, the splanchnic mesoderm cells are differentiated to esophageal/gastric mesenchyme cells in a one-step process. In these embodiments, the methods comprise contacting splanchnic mesoderm cells with a RA signaling pathway activator (e.g. RA), a HH signaling pathway activator (e.g. PMA), and a BMP signaling pathway inhibitor (e.g. Noggin). In some embodiments, the RA signaling pathway activator, the HH signaling pathway activator, and the BMP signaling pathway inhibitor of the one-step process are contacted in the concentrations described herein for a period of time sufficient to differentiate the splanchnic mesoderm cells to esophageal/gastric mesenchyme cells. In some embodiments, the splanchnic mesoderm cells are contacted with the factors described herein, e.g. RA, PMA and Noggin, for a period of time sufficient to differentiate the splanchnic mesoderm cells to esophageal/gastric mesenchyme cells. In some embodiments, the contacting is for a period of time that is, is about, is at least, is at least about, is not more than, or is not more than about, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 79, 80, 81, 82, 83, 84, 85, 86, 87, 88, 89, 90, 91, 92, 93, 94, 95, 96, 97, 98, 99, 100, 101, 102, 103, 104, 105, 106, 107, or 108 hours, or any period of time within a range defined by any two of the aforementioned times. In some embodiments, the contacting is for a period of time that is, is about, is at least, is at least about, is not more than, or is not more than about, 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times. In some embodiments, the contacting is for a period of time that is, is about, is at least, is at least about, is not more than, or is not more than about, 72 hours.

In some embodiments, the splanchnic mesoderm cells are differentiated into esophageal/gastric mesenchyme cells in a two-step process. In these embodiments, the methods comprise a first step of contacting the splanchnic mesoderm cells with a RA signaling pathway activator and a HH signaling pathway activator prior to a second step prior to a second step of contacting the splanchnic mesoderm cells with a RA signaling pathway activator, a HH signaling pathway activator, and a BMP signaling pathway inhibitor (e.g. Noggin). In some embodiments, the RA signaling pathway activator (e.g. RA) and the HH signaling pathway activator (e.g. PMA) of the first step and the second step are the same. In some embodiments, the RA signaling pathway activator and the signaling pathway activator of the first step and the second step are different. In some embodiments, the RA signaling pathway activator (e.g. RA) and the HH signaling pathway activator (e.g. PMA) of the first step, and the RA signaling pathway activator (e.g. RA), the HH signaling pathway activator (e.g. PMA), and the BMP signaling pathway inhibitor (e.g. Noggin) of the second step are contacted in the concentrations described herein for a period of time sufficient to differentiate the splanchnic mesoderm cells to respiratory mesenchyme cells. In some embodiments, the splandinic mesoderm cells are contacted with the factors described herein, e.g. RA, PMA and Noggin, for a period of time sufficient to differentiate the splanchnic mesoderm cells to esophageal/gastric mesenchyme cells. In some embodiments, the RA signaling pathway activator (e.g. RA) and the HH signaling pathway activator (e.g. PMA) of the first step are contacted for a period of time that is, is about, is at least, is at least about, is not more than, or is not more than about, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 ,43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times. In some embodiments, the RA signaling pathway activator (e.g. RA) and the HH signaling pathway activator (e.g. PMA) of the first step are contacted for a period of time that is, is about, is at least, is at least about, is not more than, or is not more than about, 36, 37, 38, 39, 40, 41, 42, 43, 44, 45, 46, 47, 48, 49, 50, 51, 52, 53, 54, 55, 56, 57, 58, 59, or 60 hours, or any period of time within a range defined by any two of the aforementioned times. In some embodiments, the RA signaling pathway activator (e.g. RA) and the HH signaling pathway activator (e.g. PMA) of the first step are contacted for a period of time that is, is about, is at least, is at least about, is not more than, or is not more than about, 48 hours. In some embodiments, the RA signaling pathway activator (e.g. RA), the HH signaling pathway activator (e.g. PMA), and the BMP signaling pathway inhibitor (e.g. Noggin) of the second step are contacted for a period of time that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45. 46, 47, or 48 hours, or any period of time within a range defined by any two of the aforementioned times. In some embodiments, the RA signaling pathway activator (e.g. RA), the HH signaling pathway activator (e.g. PMA), and the BMP signaling pathway inhibitor (e.g. Noggin) of the second step are contacted for a period of time that is, is about, is at least, is at least about, is not more than, or is not more than about, 12, 13, 14, 15, 16, 17, 18, 19, 20, 21, 22, 23, 24, 25, 26, 27, 28, 29, 30, 31, 32, 33, 34, 35, or 36 hours, or any period of time within a range defined by any two of the aforementioned times. In some embodiments, the RA signaling pathway activator (e.g. RA), the HH signaling pathway activator (e.g. PMA), and the BMP signaling pathway inhibitor (e.g. Noggin) of the second step are contacted for a period of time that is, is about, is at least, is at least about, is not more than, or is not more than about, 24 hours.

In some embodiments, the esophageal/gastric mesenchyme cells exhibit increased expression of MSC, BARX1, WNT4, HOXA1, FOXF1, or NKX3-2, or any combination thereof, relative to cardiac endoderm cells, splanchnic mesoderm cells, or respiratory mesenchyme cells, or any combination thereof. In some embodiments, the esophageal/gastric mesenchyme cells exhibit decreased expression of WNT2, TBX5, MSX1, MSX2, or LHX2, or any combination thereof, relative to splanchnic mesoderm cells, septum transversum cells, fibroblasts, or respiratory mesenchyme cells, or any combination thereof.

Factors for Differentiating Splanchnic Mesoderm

In any of the embodiments provided herein, the splanchnic mesoderm cells are contacted with a RA signaling pathway activator. In some embodiments, the RA signaling pathway activator is selected from the group consisting of retinoic acid, all-trans retinoic acid, 9-cis retinoic acid, CD437, EC23, BS 493, TTNPB, or AM580. In some embodiments, the RA signaling pathway activator is or comprises RA. In some embodiments, the splanchnic mesoderm cells are contacted with the RA signaling pathway activator at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM. In some embodiments, the splanchnic mesoderm cells are not contacted with a RA signaling pathway activator.

In any of the embodiments provided herein, the splanchnic mesoderm cells are contacted with a BMP signaling pathway activator. In some embodiments, the BMP signaling pathway activator is selected from the group consisting of BMP1, BMP2, BMP3, BMP4, BMP5, BMP6, BMP7, BMP8a, BMP8b, BMP10, BMP11, BMP15, IDE1 and IDE2. In some embodiments, the BMP signaling pathway activator is or comprises BMP4. In some embodiments, the splanchnic mesoderm cells are contacted with the BMP signaling pathway activator at a concentration that is, is about, is at least, is at least about, is not more than, or is not more than about, 1, 5, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, for example, 1 to 100 ng/mL, 5 to 40 ng/mL, 10 to 80 ng/mL, 1 to 50 ng/mL, or 50 to 100 ng/mL. In some embodiments, the splanchnic mesoderm cells are not contacted with a BMP signaling pathway activator.

In any of the embodiments provided herein, the splanchnic mesoderm cells are contacted with a Wnt signaling pathway activator. In some embodiments, the Wnt signaling pathway activator is selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHM 98014, AZD2858, BIO, AR-A014418, SB 216763, SB 415286, aloisine, indirubin, alsterpaullone, kenpaullone, lithium chloride, TDZD 8, and TWS119. In some embodiments, the Wnt signaling pathway activator is or comprises CHIR99021. In some embodiments, the splanchnic mesoderm cells are contacted with the Lunt signaling pathway activator at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 71 to 15 μM, or 10 to 20 μM. In some embodiments, the splanchnic mesoderm cells are not contacted with a Wnt signaling pathway activator.

In any of the embodiments provided herein, the splanchnic mesoderm cells are contacted with a HH signaling pathway activator. In sonic embodiments, the HH signaling pathway activator is selected from the group consisting of SHH, IHH, DHH, PMA, GSA 10, and SAG. In sonic embodiments, the HH signaling pathway activator is or comprises PMA. In sonic embodiments, the splanchnic mesoderm cells are contacted with the HH signaling pathway activator at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM. In some embodiments, the splanchnic mesoderm cells are not contacted with a HH signaling pathway activator.

In any of the embodiments provided herein, the splanchnic mesoderm cells are contacted with a BMP signaling pathway inhibitor. In some embodiments, the BMP signaling pathway inhibitor is selected from the group consisting of Noggin, RepSox, LY364947, LDN193189, and SB431542. In some embodiments, the BMP signaling pathway inhibitor is or comprises Noggin. In some embodiments, the splanchnic mesoderm cells are contacted with the BMP signaling pathway inhibitor at a concentration that is, is about, is at least, is at least about, is not more than, or is not more than about, 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, or 250 ng/mL, or any concentration within a range defined by any two of the aforementioned concentrations, for example, 1 to 250 ng/mL, 5 to 150 ng/mL, 10 to 100 ng/mL, 1 to 150 ng/mL, or 50 to 250 ng/mL. In some embodiments, the splanchnic mesoderm cells are not contacted with a BMP signaling pathway activator.

In any of the embodiments provided herein, the splanchnic mesoderm cells are contacted with one or more signaling pathway activators or signaling pathway inhibitors to differentiate the splanchnic mesoderm cells to splanchnic mesoderm subtypes for a period of time that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 26, 27, 28, 29, 30, 31, 32, 33, 34, 35, 36, 37, 38, 39, 40, 41, 42, 43, 44, 45, 46, 47, or 48 hours, or 1, 2, 3, 4, 5, 6, 7, 8, 9, 10, 11, 12, 13, 14, 15, 16, 17, 18, 19, or 20 days.

Also disclosed herein are any one of the splanchnic mesoderm cells produced by any one of the methods provided herein. Also disclosed herein are any of the septum transversum cells produced by any one of the methods provided herein. Also disclosed herein are any of the fibroblasts produced by any one of the methods provided herein. Also disclosed herein are any of the respiratory mesenchyme cells produced by any one of the methods provided herein. Also disclosed herein are any of the esophageal/gastric mesenchyme cells produced by any of the methods provided herein.

### EXAMPLES

Some aspects of the embodiments discussed herein are disclosed in further detail in the following examples, which are not in any way intended to limit the scope of the present disclosure. Those in the art will appreciate that many other embodiments also fall within the scope of the disclosure, as it is described herein and in the claims.

### Example 1. Single Cell Transcriptomes Define Progenitor Diversity in the Developing Foregut

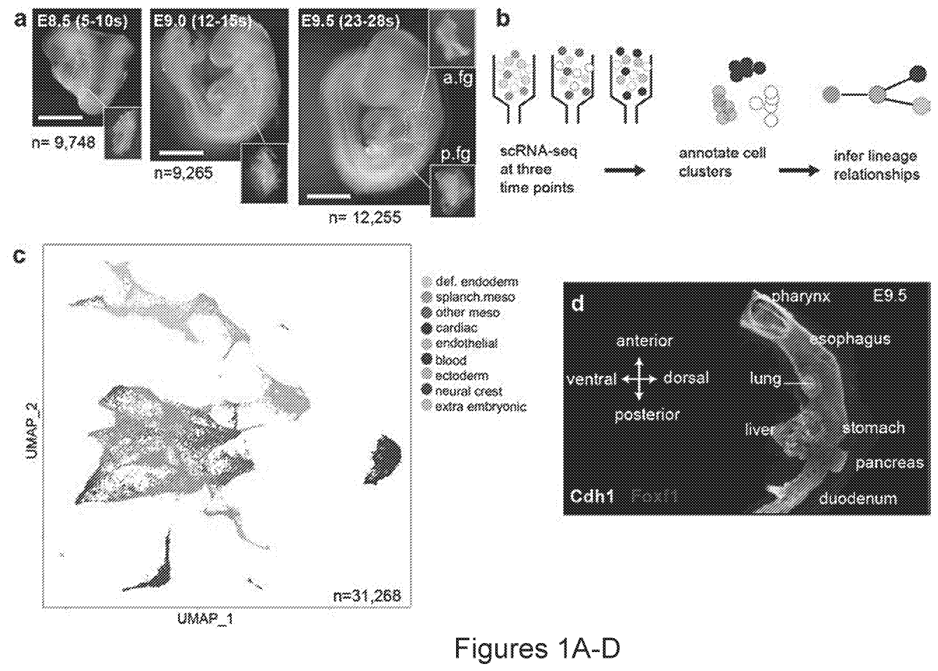

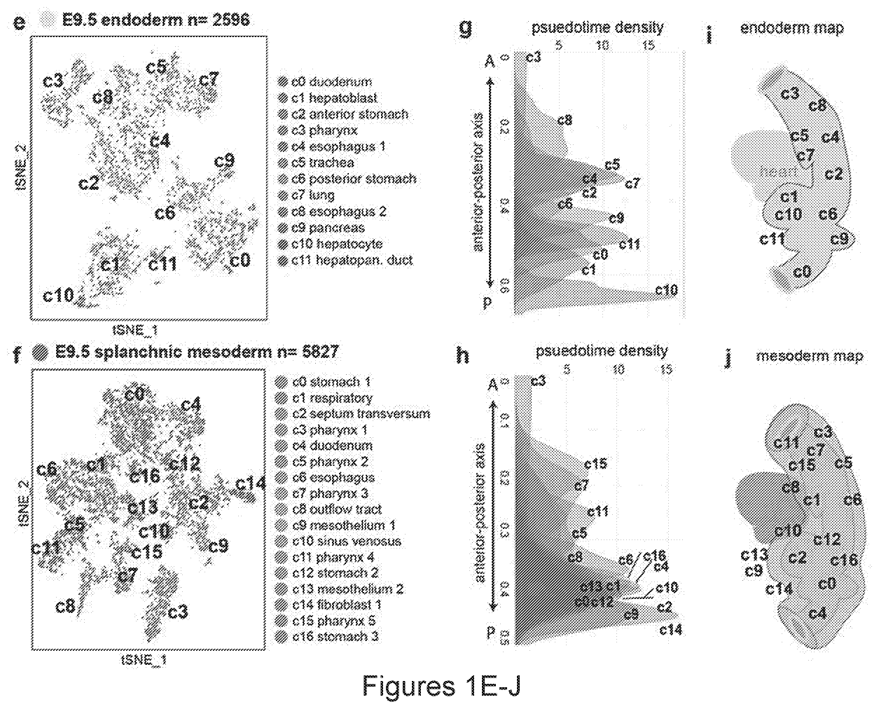

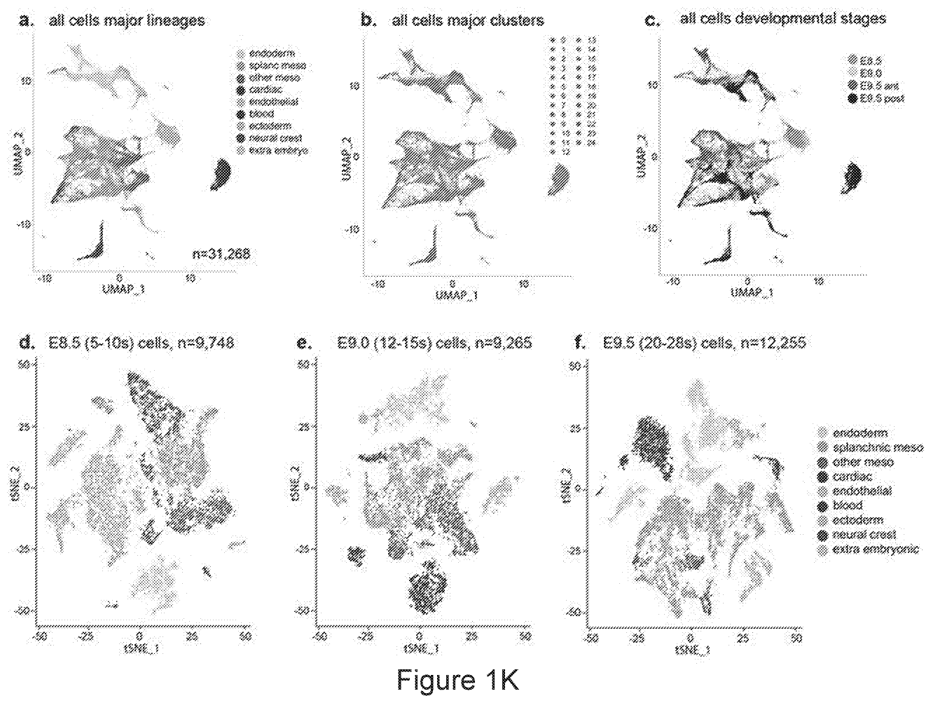

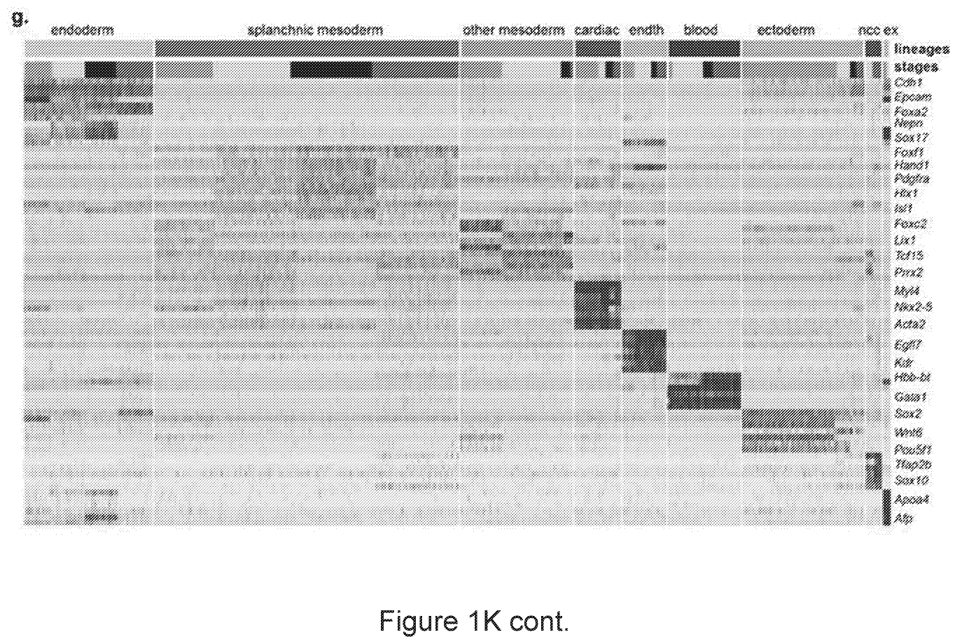

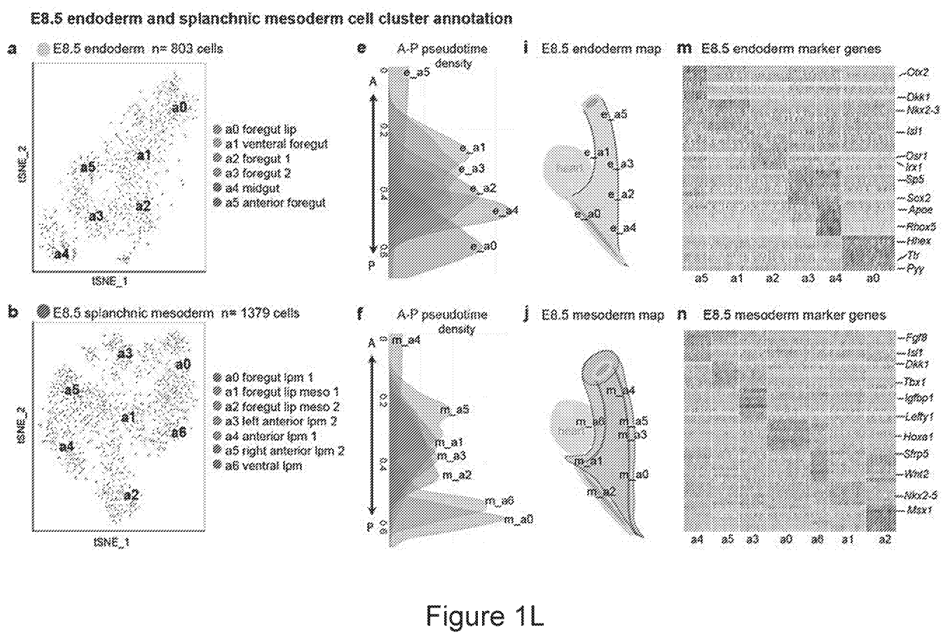

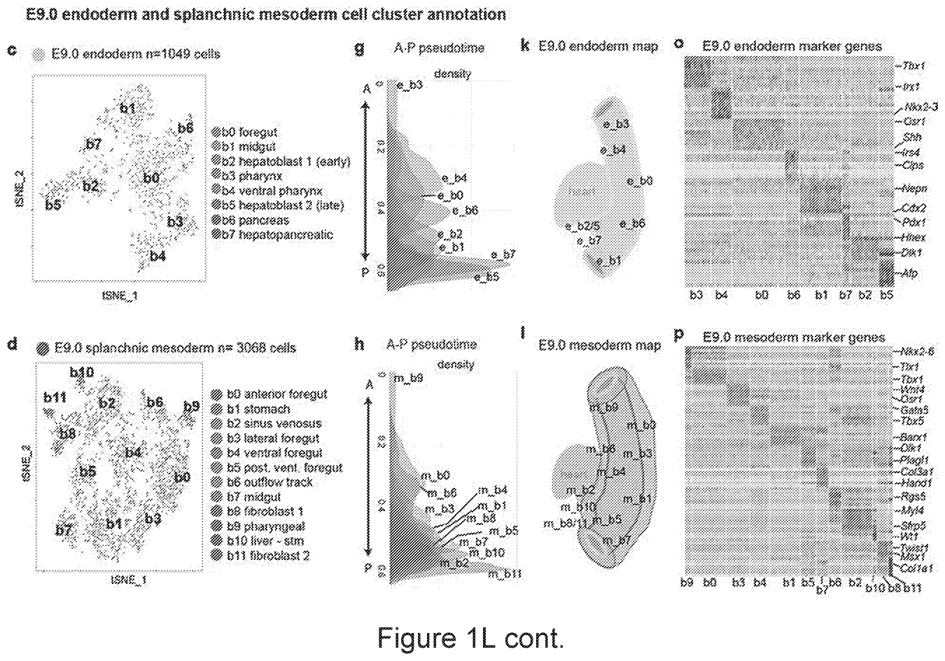

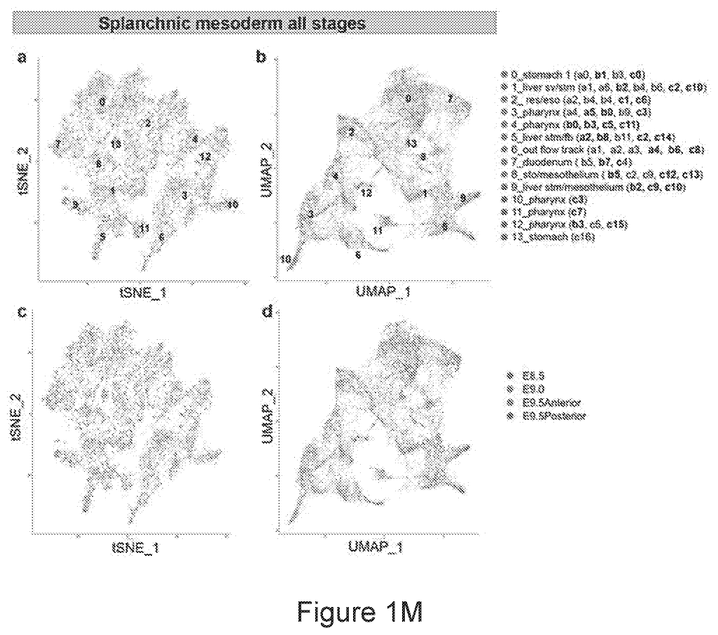

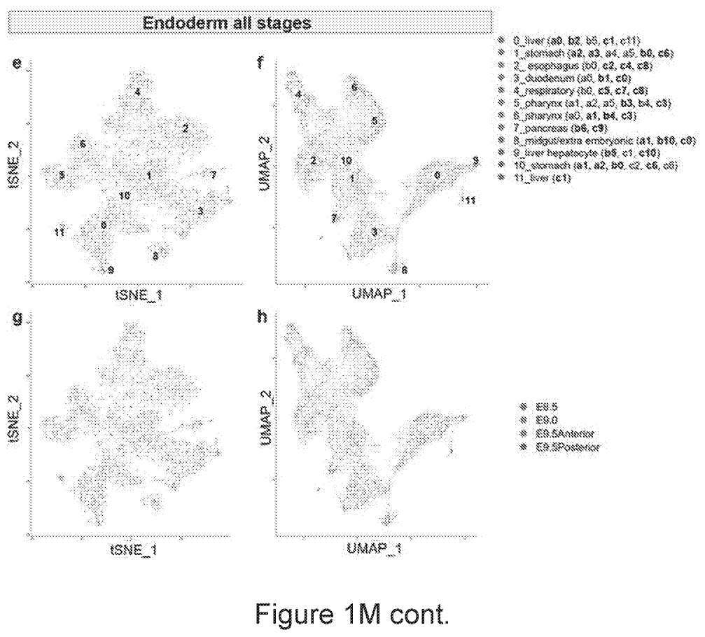

To comprehensively define lineage diversification during foregut organogenesis, single cell RNA sequence (scRNA-seq) of the mouse embryonic foregut was performed at three time points that span the period of early patterning and lineage induction: E8.5 (5-10 somites [s]), E9.0 (12-15s) and E9.5 (25-30s) (FIGS. 1A-B). The foregut was micro-dissected between the posterior pharynx and the midgut, pooling tissue from 15-20 embryos for each time point. At E9.5, anterior and posterior regions, containing lung/esophagus and liver/pancreas primordia, respectively, was isolated. A total of 31,268 single-cell transcriptomes passed quality control measures with an average read depth of 3,178 transcripts/cell. Cells were clustered based on the expression of highly variable genes across the population and visualized using uniform manifold approximation projection (UMAP) and t-distributed stochastic neighbor embedding (t-SNE) (FIGS. 1C, 1K). This identified 24 cell clusters that could be grouped into 9 major cell lineages based on well-known marker genes: DE, SM, cardiac, other mesoderm (somatic and paraxial), endothelium, blood, ectoderm, neural crest and extraembryonic (FIG. 1K). DE clusters (4,448 cells) were characterized by co-expression of Foxa1/2, Cdh1 and/or Epcam, whereas SM (10.097 cells) was defined by co-expression of Foxf1 (FIG. 1D), Vim and/or Pdgfra as well as being negative for cardiac and other mesoderm specific transcripts.

To pinpoint lineage diversification in the DE and SM, these cells were selected in silico for further analysis. 11 major DE clusters consisting of 26 stage-specific sub-clusters (E9.5, 12 clusters; E9.0, 8 clusters; E8.5, 6 clusters) and 13 major SM groups comprised of 36 stage-specific sub-clusters (E9.5, 17 clusters; E9.0, 12 clusters; E8.5, 7 clusters) were identified (FIGS. 1E-F, 1L,-M). Clusters were annotated by comparing their distinguishing genes with published expression patterns of over 160 genes in the Mouse Genome Informatics (MGI) database. These data provide a comprehensive single cell resolution view of early foregut organogenesis and can be explored at research.cchmc.org/ZornLab-singlecell of the World Wide Web.

The annotations identified all the major DE organ lineages at E9.5 including: Tbx1+ pharynx, two Nkx2-1/Foxa2+ respiratory clusters, two Sox2+ esophagus clusters, two Sox2/Osr1+ stomach clusters, two Alb/Prox1/Afp+ hepatic clusters (c1_hepatoblasts and c10_early hepatocytes with higher Alb/HNF4a expression). Sox17/Pdx1+ hepatopancreatic duct, Pdx1/Mnx1+ pancreas and Cdx2+ duodenum (FIG. 1E). Consistent with the dissections, Nkx2-1+/Hhex+ thyroid progenitors were not detected. Similar to recent scRNA-seq analysis of the E8.75 gut epithelium, half a dozen distinct DE progenitor states between E8.5 and E9.0 were also annotated based on the restricted expression of lineage specifying transcription factors (TFs), including Otx2+ anterior foregut, Sox2/Sp5-enriched dorsal lateral foregut, Osr1/Irx1-enriched foregut, Hhex+ hepatic endoderm, Nkc2-3+ ventral DE adjacent to heart and a small population of Cdx2+ midgut cells (FIG. 1L).

### Example 2. Validation of Novel Mesenchymal Subtypes

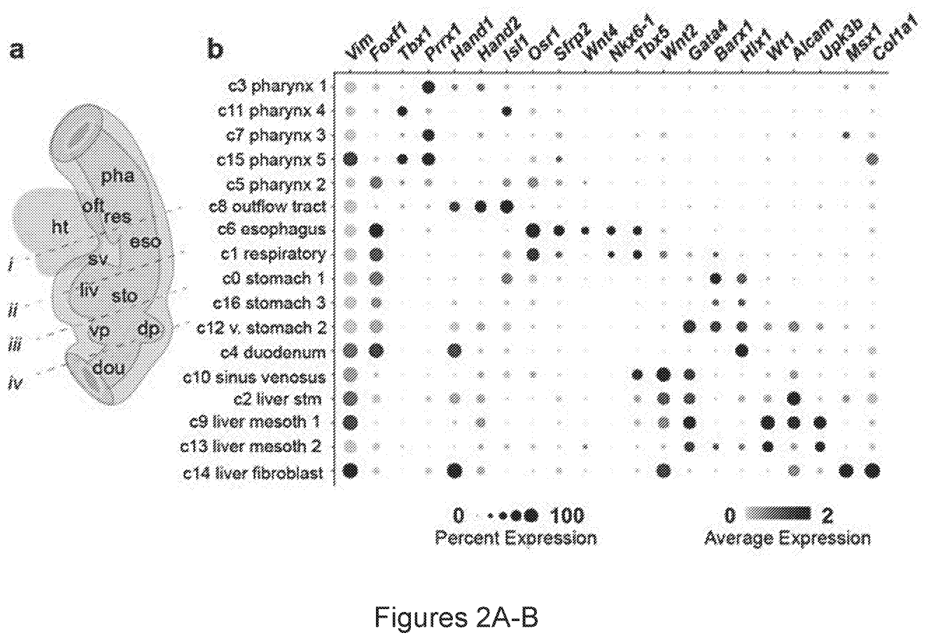

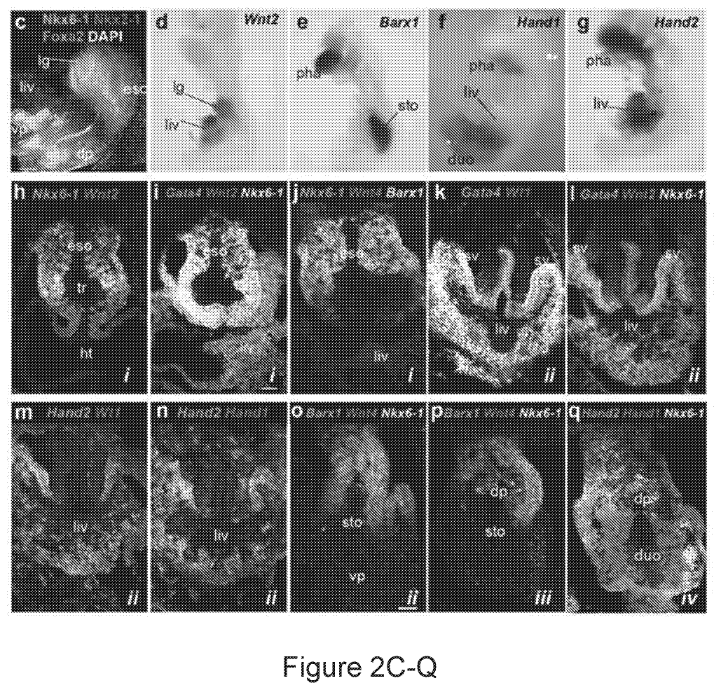

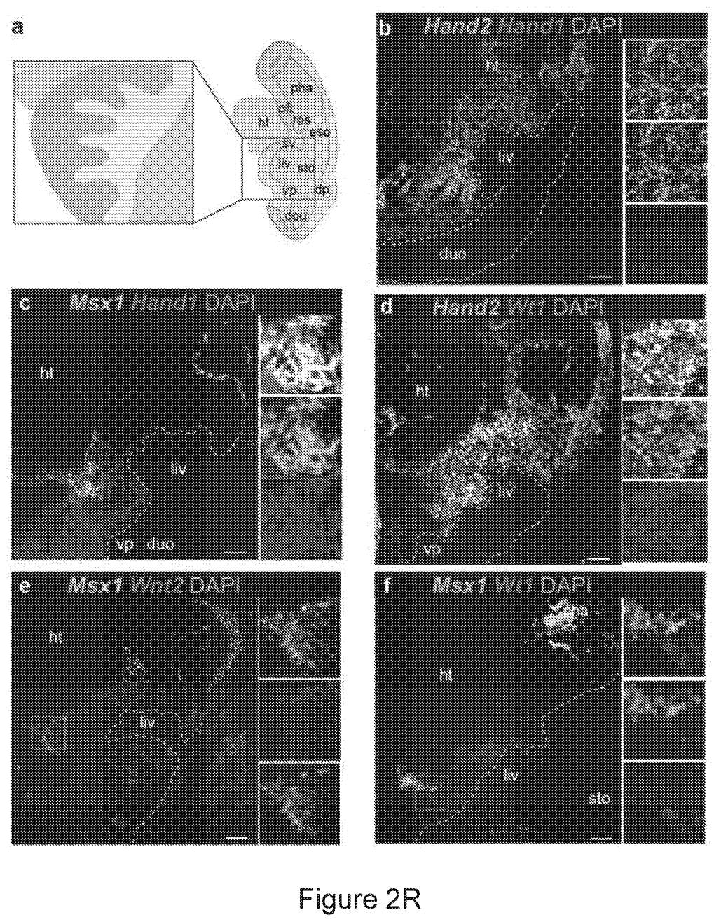

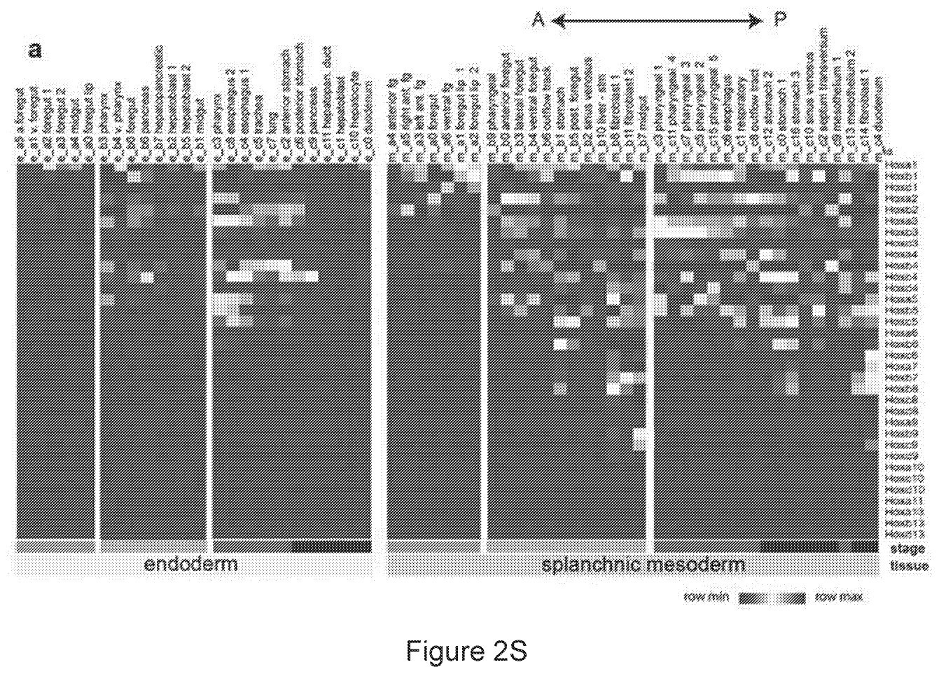

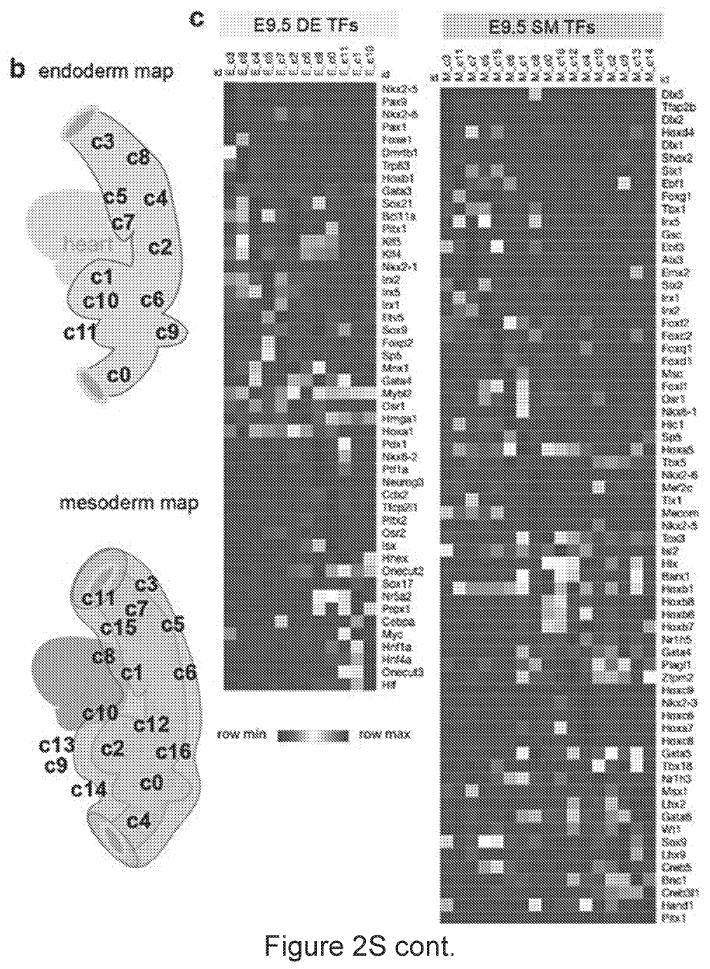

At all stages, the SM cell type diversity in the foregut was surprisingly complex, much more than previously appreciated (FIGS. 1F, 1L). However, unlike the DE, SM populations were typically defined not just by one or two markers, but rather by a combination of multiple transcripts (FIGS. 2A-B). In situ hybridization and immunostaining of E9.5 foreguts and embryo sections confirmed that combinations of co-expressed transcripts defined different organ-specific SM subtypes (FIGS. 2C-Q). The 17 SM cell populations at E9.5 included five Tbx1/Prrx1+ pharyngeal clusters, Isl1/Mtus2+ cardiac outflow tract cells, Nkx6-1/Gata4/Wnt2+ respiratory and Nkx6-1/Sfrp2/Wnt4+ esophageal mesenchyme (FIGS. 2B-J). Three Barx1/Hlx+ stomach mesenchyme populations were annotated (where one was likely ventral based on Gata4 expression) and one Hand1/Hoxc8+ duodenum mesenchyme. Pancreas-specific mesenchyme was not identified and was suspected to be in the stomach or duodenum clusters (FIGS. 2P-Q).

Unexpectedly, the liver bud had five distinct mesenchymal populations. Data mining of MGI and in situ validation allowed for annotation of an A;ca,/Wnt2/Gata4-enriched stm, a Tbx5/Wnt2/Gata4/Vsn11+ sinus venosus, a Msx1/Wnt2/Hand1/Colla1+ fibroblast population and two Wtl/Gata4/Uroplakin+ mesothelium populations (FIGS. 2K-N, 2R). Interestingly, the restricted expression of Hand1 and Hand2 in the posterior versus anterior liver bud (FIG. 2R, panel b) and the mutually exclusive expression of Msx1 from Wnt2 and Wt1 (FIG. 2R panel e-f) was observed.

### Example 3. Pseudotime Spatial Ordering of Foregut Cells

Different organs form at precise locations along the anterior-posterior (A-P) axis of the gut. To assess whether this was reflected in the single cell transcriptional profiles, a pseudotitne analysis, which have been used to examine positional information of cells in a continuous field of embryonic tissue, was employed. To this end, the DE and SM cells were analyzed at each stage using diffusion maps, a dimensional reduction method for reconstructing developmental trajectories. Anchoring the most anterior pharyngeal cluster as a root, the pseudotime density distribution for each cluster was plotted based on transition probabilities from root cells to all other cells in the graph. Remarkably, this ordered both the DE and SM cell populations according to their appropriate A-P position in the embryo, indicating that the analysis represents an unbiased proxy of pseudo-space (FIG. 1G-J, 1L). The data also indicated that at this time in development, cells in the embryonic gut tube exhibit a continuum of transcriptional signatures of which spatially adjacent cell types having more similar expression profiles than distant cell types. Indeed, the E9.5 clusters from the anterior dissections were located in the anterior half of the pseudo-space continuum, compared to posterior tissue, confirming the robustness of the computational ordering. Finally, Hox genes, which are known to be expressed in a co-linear fashion along the A-P axis, was examined and a progressive increase in posterior Hox paralog expression in more posterior clusters, particularly within the SM, was observed (FIG. 2S).

Combining the pseudo-space analysis, MGI curations and in sun validation, each DE and SM population was mapped to their approximate locations in the gut tube (FIGS. II-J, 1L). This revealed that the SM diversity mirrored DE lineages, indicating their closely coordinated development from the very beginning of organogenesis.

### Example 4. Transcription Factor Code of Foregut Endoderm and Mesenchyme

DE organ lineages have historically been defined by the overlapping expression domains of a few transcription factors (TFs). While some regionally expressed TFs have been reported in the SM, the single cell RNA-seq data allows for defining a comprehensive combinatorial code of differentially expressed TFs that distinguish different SM and DE subtypes (FIGS. 2S). This revealed new lineage-restricted markers such as homeodomain IF Nkx6-1. Well known for its expression in the pancreatic endoderm (FIG. 2P), Nkx6-1 was also specifically expressed in the respiratory and esophageal mesoderm at E9.5 (FIGS. 213-C, H-J). This IF code facilitates lineage tracing experiments and studies testing their role in mesenchymal differentiation.

### Example 5. Synchronized Endoderm and Mesenchyme Lineage Trajectories

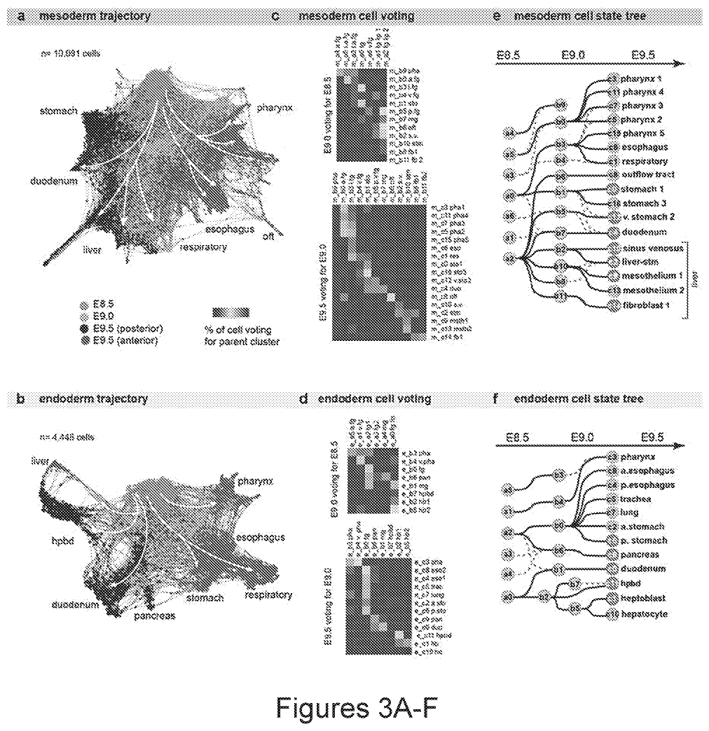

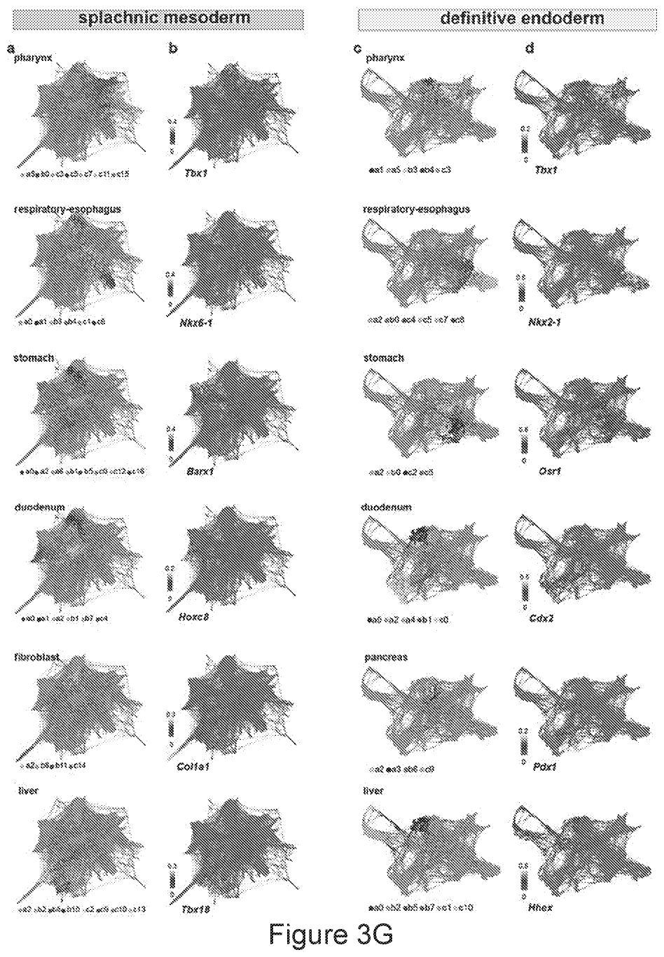

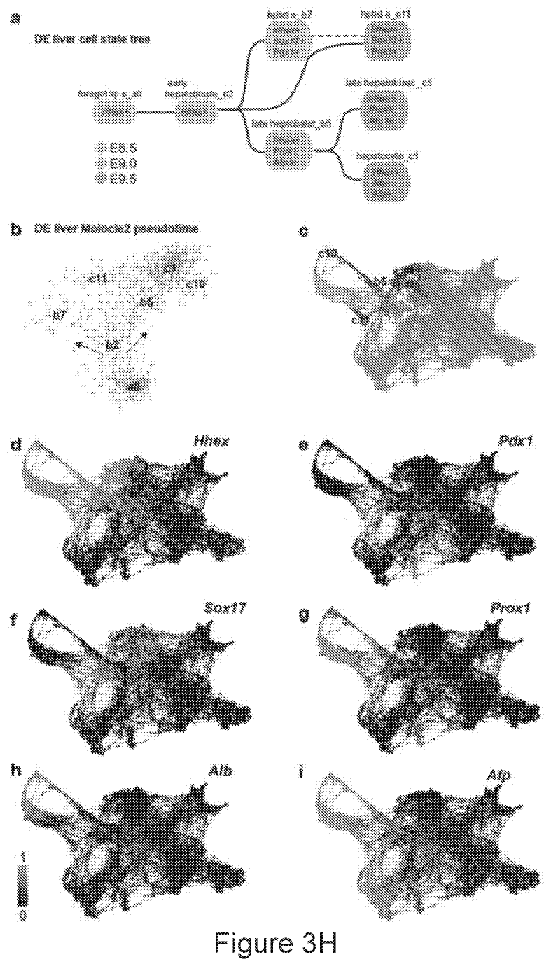

The transcriptional cell state complexity of the DE and SE doubled in just 24 hours between E8.5 and E9.5, reflecting progenitors forming more specialized cell types. To examine the temporal dynamics of lineage diversification, the single cell data was visualized using SPRING (FIGS. 3A-B), an algorithm that represents k-nearest neighbors in a force directed graph, facilitating analysis of developmental trajectories. Both the DE and SM trajectories progressed from a continuum of closely related cell states at E8.5 to transcriptionally distinct cell populations at E9.5 (FIGS. 3A-B, 3G), consistent with the transition from multipotent progenitors to organ specific lineages. Importantly, the cell clusters defined by tSNE were well-preserved in SPRING (FIG. 3G), supporting the robustness of the clustering. One striking observation evident in the structure of the SPRING plots was the apparent coordination of SM and DE lineage diversification over the 24 hours.

To more clearly visualize the developmental trajectories associated with lineage diversification, a consensus cell state tree using a single cell voting method was generated, where each cell of a later time point votes for its most likely parent of the previous time point based on gene expression similarity. All of the cell votes are then tabulated for each cluster (FIGS. 3C-D) and this is represented in a simple tree manifold (FIGS. 3E-F). While SM migration bringing distant cell types to a given organ cannot be ruled out, the data supported the notion of transcriptionally related cell states arising from the subdivision of common progenitor populations. Given that the time points were generated from pooled embryos of slightly different ages, it was possible that parent-child relationships could exist within a given time point. To address this and confirm the single cell voting results, each trajectory was assessed with a psuedotime analysis that computationally predicts progenitor states in a cell population (Monocle; Cao. J. et al. The single-cell transcriptional landscape of mammalian organogenesis. Nature 566, 496-502 (2019)). In general, the pseudotime analysis agreed with the single cell voting. But in the case of the liver endoderm, Monocle predicted a parent-child relationship within E9.0, where Hhex+ posterior foregut endoderm (cluster e_b2) gives rise to both Prox1/Afp+ hepatoblasts (e_b5) and Prox1/Sox17/Pdx1+ hepatopancreatic biliary progenitors (e_b7) (FIG. 3H), consistent with in vivo lineage tracing experiments.

Overall, the DE trajectories inferred by the single cell transcriptotnes are consistent with experimentally determined fate maps, demonstrating the robustness of the analysis herein and suggesting to us that the SM trajectories, which previously have not been well defined, may also represent lineage relationships. Having said that, it is cautioned that cells with this similar transcriptotnes may not necessarily be lineage-related. Indeed, there are cases where cells from different lineages such as ventral and dorsal pancreas can converge on similar transcriptional profiles. Thus, the results provided here establish a theoretical framework for future experimental analysis of foregut mesenchyme development.

### Example 6. Coordinated Development of Multi-Potent Progenitors

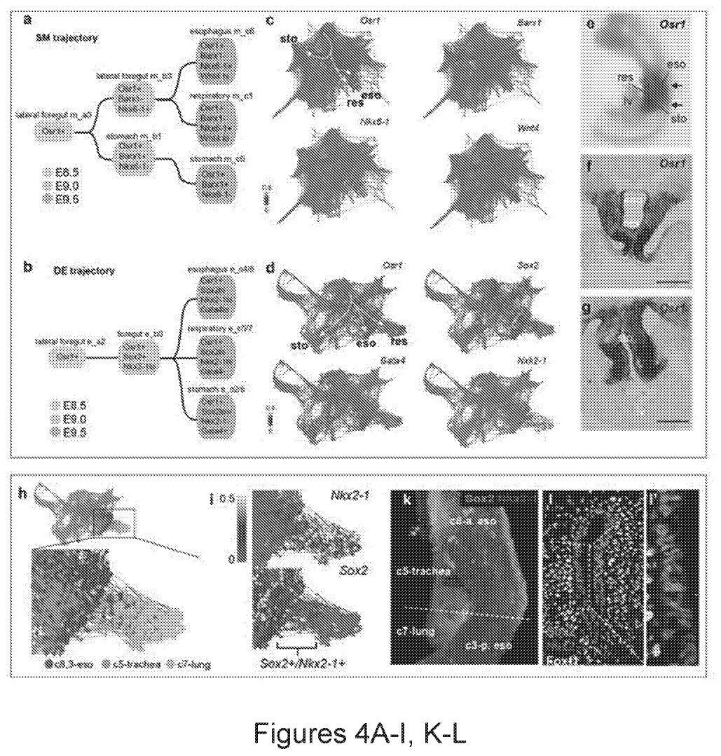

A close examination of the DE and SM trajectories suggests to us that the coordinated development from multipotent progenitors within adjacent endoderm and mesoderm tissue layers. For example, at E8.5, the DE lateral foregut cells (e_a2) and the spatially neighboring SM cells (m_a0) both express the TF Osr1, and the trajectories predict that these two cell populations are multipotent progenitors, giving rise to the respiratory, esophageal and gastric epithelium and mesenchyme, respectively (FIGS. 4A-B). As development proceeds, different cell populations appear to be segregated as they progressively express distinct lineage regulating TFs and growth factors (FIGS. 4A-D). In situ validation confirmed that Osr1 is expressed in both the epithelium and mesenchyme of the presumptive esophagus, lung and stomach at E9.5 (FIGS. 4E-G)

Furthermore, a close examination of the DE trachea cluster suggested to us a transitional cell population co-expressing the respiratory marker Nkx2-1 and the esophageal marker Sox2 at E9.5 when the foregut is being patterned along the dorsal-ventral axis (FIGS. 4H-I). Immunostaining confirmed that this was indeed a rare Nkx2-1/Sox2+ cell population at the prospective tracheal-esophageal boundary (FIGS. 4K-L), which recent studies have demonstrated to be critical in tracheoesophageal morphogenesis. In sum, the foregut lineage trajectories predicted from the single cell transcriptotnes represent a valuable resource for further studies.

### Example 7. Predicting a Signaling Road Map of Organ Induction

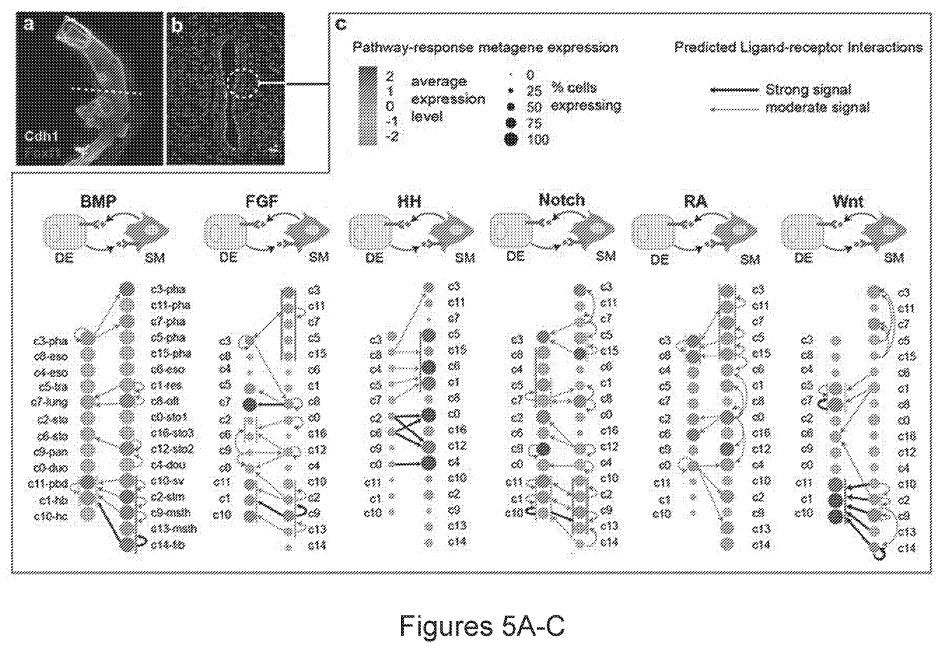

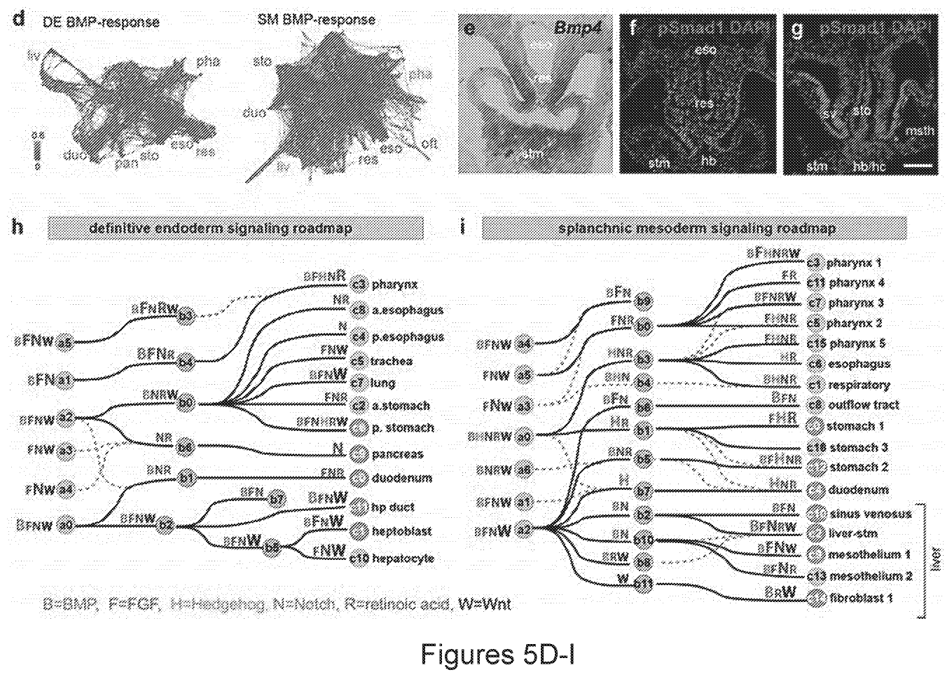

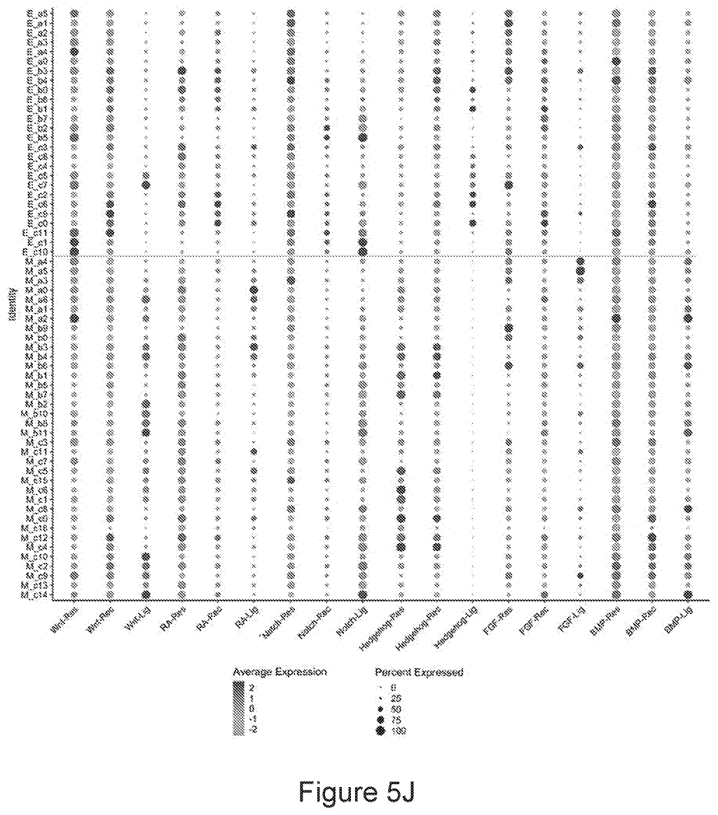

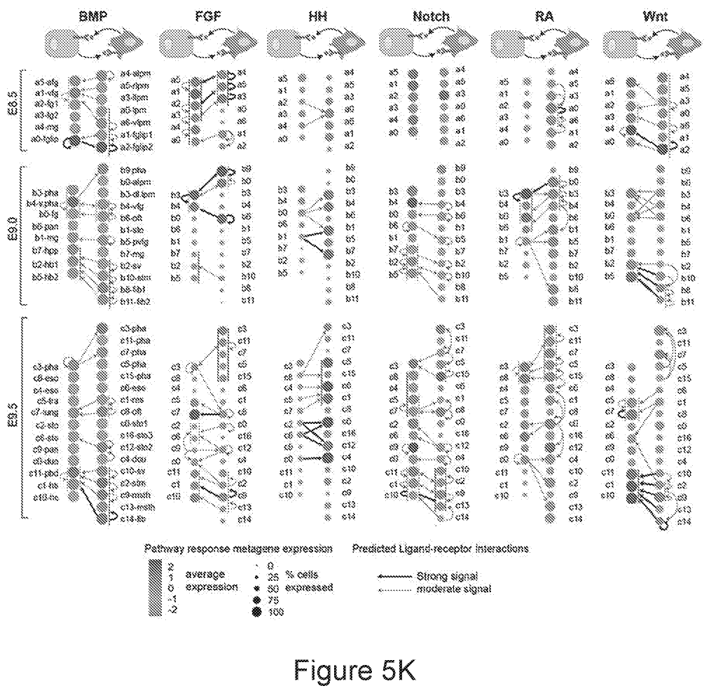

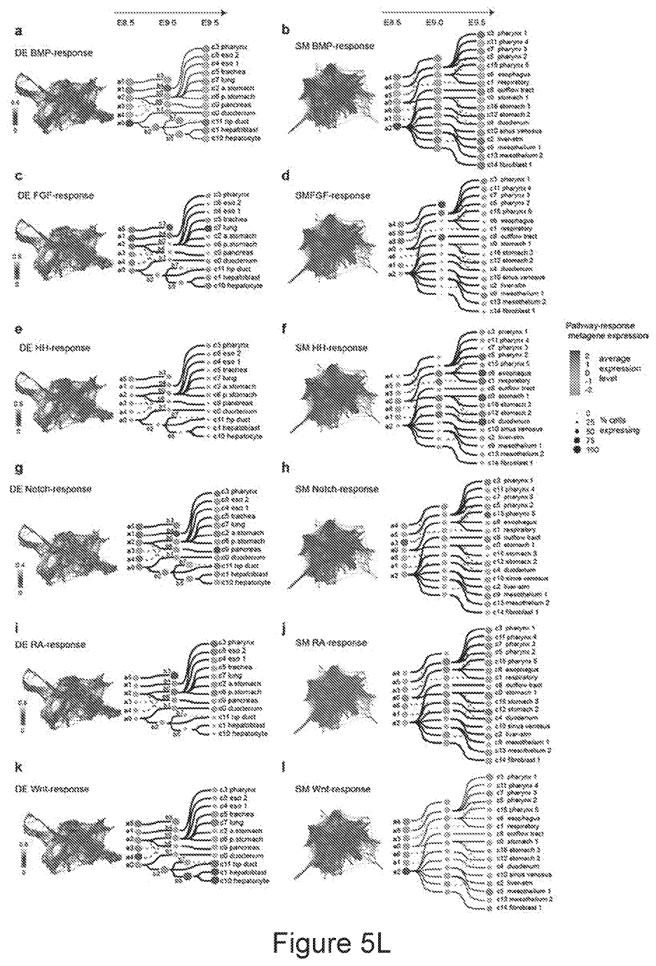

The paracrine signaling microenvironment in the foregut that controls cell fate decisions was computationally predicted (FIGS. 5A-B). Metagene expression profiles were calculated for all the ligands, receptors and context-independent response genes in each DE and SM cluster for six major signaling pathways implicated in organogenesis: BMP, FCE, Hedgehog (HH), Notch, retinoic acid (RA), and canonical Wnt (FIG. 5J). Leveraging the spatial map for each cell population in the foregut (FIG. 1I-J), cell populations along the A-P axis was ordered such that DE and SM cell types most likely to be in direct contact were opposite one another in the signaling diagram (FIG. 5C). The metagene expression levels were then used to predict potential ligand-receptor pairs and the likelihood that a given cell population was responding to local paracrine or autocrine signals (FIGS. 5A-C, 5K). The metagene expression thresholds were benchmarked on experimentally validated interactions in the literature. Also, potential ligand-receptor pairings were limited to nearby cell clusters, consistent with the generally accepted view that these pathways act over a relatively short range. Together, this analysis revealed a hypothetical combinatorial signaling network (FIGS. 5A-C, 5K).

Overall the computational predictions are consisted with known expression patterns of ligands and receptors, and identified most known signaling interactions controlling DE lineage specification. This includes mesoderm derived BMP, FGF and Wnt promoting DE liver and lung fate, and autocrine notch signaling in the DE endocrine pancreas. This suggested to us that previously undefined SM signaling predictions are also likely to be accurate. To test this, BMP signaling was examined as an example. Consistent with the scRNA-seq data, in situ hybridization confirmed high levels of Bmp4 ligand expression in the stm and the respiratory mesenchyme, while immunostaining for phospho-Smad1/5/8, the cellular effector of BMP signaling, confirmed autocrine and paracrine signaling in the developing liver and respiratory mesenchyme and epithelium, respectively, as predicted (FIGS. 5E-G).

The signaling response-metagene expression levels were projected onto the SPRING plots and cell state tree, which revealed spatiotemporally dynamic signaling domains that correlated with cell lineages (FIGS. 5D, 5L). In general, the transcriptome data predicts locally restricted interactions, with the SM being the primary source of BMP, FCE, RA and Wnt ligands, signaling to both the adjacent DE and within the SM itself (FIG. 5C). In contract, HH ligands are produced by the DE and signal to the gut tube SM, with no evidence of autocrine activity in the DE (FIG. 5C). Combining the data for all six signaling pathways onto the cell state trees, a comprehensive roadmap of the combinatorial signals predicted to coordinate the temporal and spatial development of each DE and SM lineage was generated (FIGS. 5H-I). This analysis predicted a number of previously unappreciated signaling interactions and represents a hypothesis generating resource for further experimental validation.

### Example 8. Testing the Role of Epithelial Hedgehog Signaling in Foregut Mesenchyme Patterning

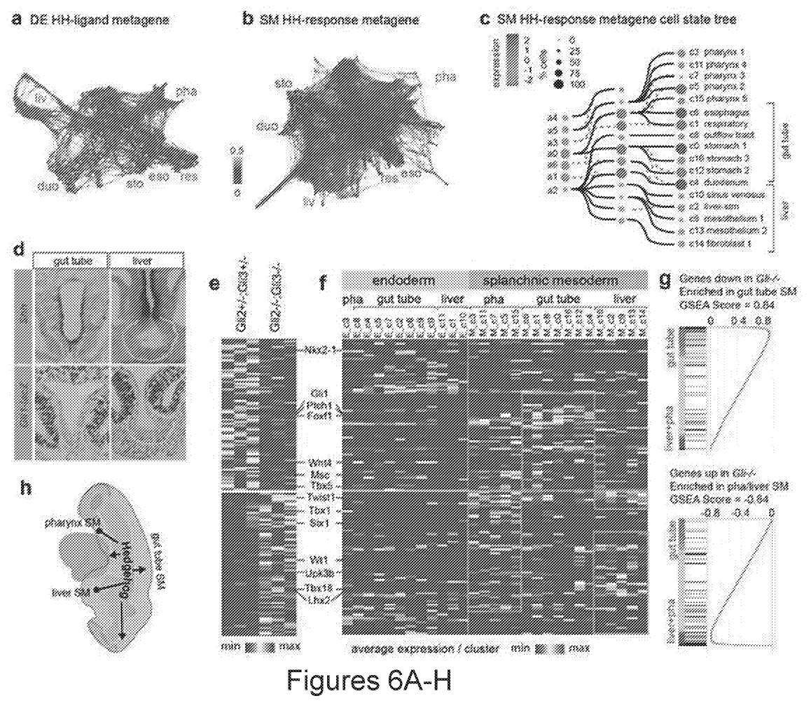

To genetically test the predictive value of the signaling roadmap, HH activity, which is suggested by the scRNA-seq to be high in gut tube SM (esophagus, respiratory, stomach and duodenum) but low in the pharyngeal and liver SM, was examined (FIGS. 6A-C). HH ligands stimulate the activation of Gli2 and Gli3 TFs, which in turn promote the transcription of HH-target genes (e.g. Gli1). Mouse embryo sections confirmed that Shh ligand was expressed in the gut tube DE with high levels of Gli1-LacZ expression in the adjacent SM. By contrast, the hepatic endoderm did not express Shh and the hepatic SM had very few if any Gli1-LacZ positive cells (FIG. 6D). To define the function of HH in SM patterning, bulk RNA-seq was performed on foreguts from Gli2−/−; Gli3−/− double mutant embryos, which lack all HH activity and fail to specify respiratory fate. Comparing homozygous mutants to heterozygous littermates, 156 HH/Gli-regulated transcripts were identified (FIG. 6E). Given the caveat that this bulk RNA sequencing is performed with both endoderm and mesoderm, the enrichment of these HH-regulated transcripts was examined in the transcriptome of DE and SM single cell clusters. This revealed that most transcripts were expressed in the SM compared to the DE. Importantly, transcripts downregulated in Gli2/3-mutants (n=80) were normally enriched in the gut tube SM, whereas upregulated transcripts (n=76) were normally enriched in the liver or pharyngeal SM (FIG. 6E-G). Interestingly, HH/Gli-regulated transcripts, including downregulated TFs (Osr1, Thx4/5, Foxf1/2) and upregulated TFs (Tbx18, Lhx2 and Wt1) have been implicated in respiratory and hepatic development respectively (FIG. 6E). This genetic analysis confirmed the predictive value of the signaling roadmap Where differential HH activity promotes gut tube versus liver and pharyngeal SM (FIG. 5I), in part by regulating other lineage specifying TFs and signaling proteins.

The data provided herein suggested to us a model where the reciprocal epithelial-mesenchymal signaling network coordinates DE and SM lineages during organogenesis. In this model, SM-derived RA induces a regionally restricted expression of Shh in the DE by E9.0, which then signals back to the SM, establishing broad pharynx, gut tube and liver domains. Other SM ligands (BMP, FGF, Notch, RA and Wnt), with distinct combinations of regional expressions in these three broad domains, then progressively subdivide DE and SM progenitors in a coordinated matter. This model can be tested by cell-specific genetic manipulations.

### Example 9. Differentiation of Splanchnic Mesenchyme-Like Lineages from Human PSCs

The new SM markers and signaling roadmap disclosed herein were then used to direct the differentiation of distinct SM subtypes from human pluripotent stem cells (hPSC), which to date have been elusive. Previous studies have established protocols to differentiate hPSC into lateral plate mesoderm (lpm) and cardiac tissue. Although both the SM and heart are derived from the lfpm, the single cell data suggested to us that in the mouse, the early SM experiences more RA signaling than the early cardiac mesoderm. This was confirmed by RA-responsive RARE:lacZ transgene expression in E8.5 embryos (FIG. 7E). Accordingly, addition of RA to the lpm differentiation media on days (d) 2-4 down-regulated the cardiac markers NKX2-5, ISL1 and TBX20 and promoted the SM markers FOXF1, HOXA1, HOXA5, and WNT2 (FIG. 7B, 7E). This is consistent with the mouse scRNA-seq data which shows that E8.5 SM expresses Nkx2-5, Isl1, and Tbx20, at lower levels than the cardiac mesoderm. Examination of PAX3, PRRX1, and CD31 confirmed that the d4 SM cultures did not express significant levels of endothelial, somatic or limb mesenchyme markers (FIG. 7E).

Next, the primitive SM was treated with different combinations of HH, RA, Wnt and BMP agonists or antagonists from d4-d7 (FIG. 7A) to drive organ-specific SM-like lineages based on the roadmap. As predicted, the HH-agonist promoted gut tube identity and efficiently blocked the hepatic fate. In the HH treated cultures, addition of RA and BMP4 (RA/BMP4) followed by WNT on d6-7 promoted gene expression consistent with respiratory mesenchyme (NKX6-1, TBX5, and WNT2) with low levels of esophageal, gastric or hepatic markers. In contrast, addition of RA and BMP4-antagonist on d6-7 promoted an esophageal/gastric-like identity (MSC, BARX1, WNT4 and NKX3-2) (FIGS. 7B-C, 7F). In the absence of HH agonist, cells treated with RA/BMP had a gene expression profile similar to liver stns and mesothelium (WT1, TBX18, LHX2 and UPK1B), whereas RA/BMP4/WNT treated cells expressed liver-fibroblast markers (MSX1/2 and HAND1). Immunostaining and RNA-scope confirmed the RT-PCR analysis (FIG. 7C-D, 7F) showing that ˜70-80% cells in the liver stm/mesothelium-like cultures were WT1+, MSX1−, NKX6-1−, whereas the other populations appear to be around 30-40%. The remainder of cells appeared to be undifferentiated rather than an alternative lineage. These data provides evidence that the signaling roadmap inferred from the mouse scRNA-seq data can be used to direct the differentiation of different organ specific SM subtypes from hPSCs.

### Example 10. Materials and Methods

Embryo Collection and Single Cell Dissociation

All mouse experiments were performed in accordance with protocols approved by the Cincinnati Children's Hospital Medical Center Institutional Animal Care and Use Committee (IACUC). No statistical sample size estimates were performed prior to the experiment, sufficient embryos to generate the material needed for the experiments were used. No randomization was utilized as no particular treatment was performed in different groups. Timed matings were set up between C57BL/6J mice and the day where a plug was detected was considered embryonic day 0.5. Staging was validated by counting somite numbers E8.5 (5-10 somites [s]), E9.0 (12-15s), and E9.5 (25-30s) (FIG. 1A-B). The foregut between the posterior pharynx and the midgut was micro-dissected, removing most of the heart and paraxial tissue and excluding the thyroid. At E9.5, anterior and posterior regions were isolated separately, containing lung/esophagus and liver/pancreas primordia, respectively. Dissected foregut tissue was pooled from 16, 20, 18 and 15 embryos from E8.5, E9.0, E9.5 anterior, and E9.5 posterior, respectively, isolated from 2-3 litters.

Single cell dissociation by cold active protease protocol was performed as known in the art. Rapidly dissected C57BL/6J mouse embryo tissues were transferred to ice-cold PBS with 5 mM CaCl₂, 10 mg/mL of Bacillus licheniformis protease (Sigma) and 125 U/mL DNAse (Qiagen) and incubated on ice with mixing by pipet. After 7 min, single cell dissociation was confirmed with microscope. Cells were then transferred to a 15 mL conical tube, and 3 mL ice cold PBS with 10% FBS (FBS/PBS) was added. Cells were pelleted (1200 G for 5 min), and resuspended in 2 mL PBS/FBS. Cells were washed three times in 5 mL PBS/0.01% BSA (PBS/BSA) and resuspended in a final cell concentration of 100,000 cells/mL for scRNA-seq. Single cell suspensions of each stage were loaded onto the Chromium Single Cell Controller instrument (10× Genomics) to generate single-cell gel beads in emulsion. Single cell RNA-sect libraries for high-throughput sequencing were prepared using the Chromium Single Cell 5′ Library and Gel Bead Kit (10× Genomics). All samples were multiplexed together and sequenced in an Illumina HiSeq 2500. The individual performing the RNA extraction, library preparation, and sequencing steps was blinded.

Immunofluorescence Staining. In Situ Hybridization and RNAscope

Mouse embryos were harvested at indicated stages and fixed in 4% paraformaldehyde (PFA) at 4° C. for overnight. The fixed samples were washed 3 times with PBS for 10 min and the foreguts were micro-dissected when indicated. Embryos or dissected foreguts were then processed as described previously by antibody staining or processed for in situ hybridization.

For RNAscope on mouse tissue, fixed embryos were immersed in 30% sucrose/PBS overnight, embedded in OCT, cryosectioned (12 μm) onto Superfrost Plus slides (Thermo Fisher) and stored at −80° C. overnight. For RNAscope of adherent hPSC culture, cells were differentiated on Geltrex-coated u-Slide 8 well (ibid) and fixed in 4% PFA at room temperature for 30 min, Cells were dehydrated with ethanol gradient and stored in 100% ethanol at −20° C. RNAscope fluorescent in situ hybridization was conducted with RNAscope Multiplex Fluorescent Detection Reagents V2 (Advanced Cell Diagnostics, Inc.) and Opal fluorophore (Akoya Biosciences) according to manufacturer's instructions.

Pre-Processing 10× Genomics Raw scRNA-seq Data

Raw scRNA-seq data was processed using CellRanger (v2.0.0, 10× Genomics, available on the World Wide Web at github.com/10× Genomics/cellranger). Reads were aligned to mouse genome [mm10] to produce counts of genes across barcodes. Barcodes with less than ˜5k UMI counters were not included in downstream analysis. Percentage of reads mapped to transcriptotne was ˜70% each sample. The resulting data comprised 9748 cells in E8.5, 9265 cells in E9.0, 7208 cells in E9.5 anterior samples, and 5085 cells in E9.5 posterior samples.

Quality Control, Dimensionality Reduction, Clustering and Marker Prediction

Subsequent QC and clustering was performed using Seurat [v2.3.4] package in R. Basic filtering was carried out where all genes expressed ≥3 cells and all cells with at least 100 detected genes were included. QC was based on nGene and percentmito parameters to remove the multiplets and cells with high mitochondrial gene expression. After filtering, 9748, 9265 and 12255 cells were retained in E8.5, E9.0 and E9.5 samples respectively. Global scaling was used to normalize counts across all cells in each sample [scale factor:10000] and cell cycle effect was removed by regressing out difference between S phase and G2M phase from normalized data using default parameters. First, each developmental stage was clustered separately to identify major cell lineages. Approximately 1500 highly variable genes (HVG) across each population were selected by marking outliers from dispersion vs. avgExp plot. PCA was performed using HVG, and the first 20 Principal Components were used for cells clustering, which then was visualized using t-distributed stochastic neighbor embedding (tSNE). Marker genes defining each cluster were identified using ‘FindAllMarkers’ function (Wilcoxon Rank Sum Test) in Seurat and these were used to annotate clusters based on well-known cell type specific genes.

Cells from all three time points were integrated with Seurat (v3.0) using a diagonalized canonical correlation analysis (CCA) to reduce the dimensionality of the datasets followed by L2-normalization of canonical correlation vectors (CCV). Finally, mutual nearest neighbors (MNN) were obtained, which also are referred as integration anchors (cell pairs) to integrate the cells. First, 30 CCs (canonical correlation components) were used for clustering and non-linear dimension reduction approaches (UMAP and tSNE) were used to reduce the dimensions and visualize cells in two dimensions.

In Silico Selection and Clustering for Definitive Endoderm and Splanchnic Mesenchyme

Definitive endoderm (DE) clusters (4,448 cells) were defined by the co-expression of Foxa1/2, Cdh1 and/or Epcam, whereas the splanchnic SM (10.097 cells) were defined by co-expression of Foxf1, Vim and/or Pdgfra as well as being negative for cardiac, somatic and paraxial mesoderm specific transcripts. Cells from DE and SM clusters were extracted from each time point and re-clustered using Seurat [v2.3.4] to define lineage subtypes. Prior to re-clustering blood, mitochondrial, ribosomal and strain-dependent noncoding RNA genes were regressed from the data. Dimensionality reduction, clustering and marker prediction steps were performed as described above for each stage. DE and SM cell subtypes were annotated by manual curation comparing the cluster marker genes with over 300 published expression profiles in the MGI database and our own gene expression validations. DE and SM clusters from all three time points analyzed together using Seurat (v3.0) integration approach explained above respectively.

Transcription Factor Code from DE and SM Lineages

To identify TFs with enriched expression specific to different DE and SM cell types, ‘FindAllMarkers’ function in Seurat [v3.0] was utilized on set of 1623 TFs expressed in the mouse genome [AnimalTEDB]. Raw counts of TFs were normalized and scaled in Seurat [v3.0]. Cells in clusters served as replicates in finding marker TFs for each lineage. Wilcoxon rank sum test was used in identifying marker TFs. Top 5 marker TFs were then visualized using DimHeatmap function in Seurat(V3.0).

Pseudo-Time Analysis of Spatial Organization of Cell Populations

To examine whether pseudo-time analysis could inform the spatial organization of cells in the continuous sheet of tissue DE or SM tissue, a pseudo-time analysis was performed using URD [v1.0]. Firstly, in order to calculate pseudotime, transition probabilities were calculated for DE and SM cells at each stage using diffusion maps. Then, the calcDM function was used to generate diffusion map components and the first 8 components were used to calculate transition probabilities among cells. Next, to calculate pseudotime, root cells were fixed to the most anterior clusters based on manual annotation. Starting from root cells, a probabilistic breadth-first graph search using transition probabilities was performed until all the cells in the graph have been visited. Multiple simulations were run and psuedotime equaled average iteration that visited each cell in the graph from the root cells. Following functions in URD were used to calculate pseudotime (“floodPseudotitne” and “floodPsuedotimeProcess”). Finally, density distribution of pseudotime was plotted for each cluster/cell-type using plotDists function. Density distribution of psuedotime, ordered clusters similarly to the manually curated order of cell types along the A-P axis.

SPRING Analysis of Cell Trajectories

To examine cell trajectories across the three time points. SPRING [v1.0], which uses a k-Nearest Neighbors (KNN) graph (5 nearest neighbors), was implemented to obtain force-directed layout of cells and their neighbors. To understand transcriptional change across cell states (lineages), first 40 principal components (PC) were learned from the latest time point E9.5, and this PC space was used to transform the entire data set (E8.5, E9.0, and E9.5). This transformed data was used to generate a distance matrix which then was used to obtain the KNN graph using the default parameters.

Inferring a Cell-State Tree by Parent-Child Single Cell Voting

To visualize the trajectories in a simple transcriptional cell state tree, a parent-child single cell voting approach based on the KNN classification algorithm was used. First, a normalized counts matrix was generated using the distinguishing marker genes from all DE or SM clusters as features at each stage. Marker genes were used as features to train KNN, during which the KNN learns the distance among cells in the training set based on the feature expression. Each cell was classified based on the Seurat cluster assignment. Cells of a later time point vote for their most likely parent cells in the earlier time point as follows: train KNN using E8.5 cells and test by E9.0 cells voting for E8.5 cells. KNN resulted in vote probability for each cell in E9.0 against each cluster in E8.5, which was subsequently averaged for each cluster in E9.0 against each cluster in E8.5. This approach was repeated with E9.5 cells voting for E9.0 parents. The average vote probability for a given cluster was tabulated, normalized for cluster size and represented as a % of total votes in a confusion matrix. The top winning votes linking later time points back to the preceding time point were displayed as a solid line on the tree, Prominent second choices with >60% of winning votes were reported on the tree as dashed lines. This vote probability was also compared with the confusion matrix resulting from the KNN to assess the transcriptional cell-state tree. In more than 99% cases, these two methods resulted in the same first and second choices, thereby validating deduced parent-child relationships.

To validate the cell state tree assertions using pseudotinie analysis, Monocle [v3.0.0] was deployed on individual lineages/cell states. tSNE was used for Dimensionality Reduction and principle graph was learned using SimplePPT. All other parameters were set to default.

Calculation of Metagene Profiles

For six of the major paracrine signaling pathways implicated in foregut organogenesis (BMP, FGF, HH, Notch, RA, and canonical Wnt), a list of all the well-established ligands, receptors, and context-independent pathway response genes that were encoded in the mouse genome were curated. Then, “ligand-metagene”, “receptor-metagene”, and “response-metagene” profiles were calculated by summing the normalized expression of each individual gene for each pathway (e.g.: Wnt-ligand metagene=Σ(Wnt1+Wnt2+Wnt2b+Wnt3. . . Wnt10b expression)) in each cell and cluster as follows:

Assuming that there are x genes in the gene set and n cells. Gene has (a1, a2. . . an) counts, Gene2 has (b1, b2. . . bn) counts and so on.

Step 1: Each gene's counts were normalized using the gene's max count across all DE and SM cells (n=14,545 cells): Gene1_norm=(a1, a2. . . an)/max(a1, a2. . . an).

Step2: Normalized counts of genes were summed up, for each cell, to generate a metagene_v1 with counts across cells: metagene_v1=Gene1_norm+Gene2_norm+. . . +Genex_norm. Presuming summed up counts are: m1, m2. . . mn.

Step 3: Summed counts of metagene_v1 were normalized by max counts of the metagene_v1, to create a metagene profile for each cell: MetaGene=(m1, m2. . . mn)/max(m1, m2. . . mn). The average Metagene expression profiles for ligands, receptors and response genes in each DE and SM cluster were then calculated in Seurat [v3.0] using ‘AverageExpression’ function. The average expression profiles of metagene across all DE and SM clusters were visualized as a Dotplot using Seurat. Average expression of metagene expression profiles were scaled from −2 to 2 for Dotplot visualization.

Prediction of Receptor-Ligand Interactions

A given cell type was scored to be expressing enough ligand to send a signal or enough receptor to respond to ligand if the average ligand-metagene or receptor-tnetagene expression level was ≥−1 and expressed in ≥25% of cells. (Except for the Notch ligand-metagene where expression threshold of ≥−1.5 was used due to low overall expression in all cell). These thresholds empirically set to be conservative and benchmarked against experimentally validated signaling interactions in DE liver, lung and pancreas. Furthermore, the likelihood that a given cell population was responding based on the context-independent pathway response-metagene expression level being ≥−1 and expressed in ≥25% of cells was determined. Context-independent response genes are those genes that are known in the art to be directly transcribed in most cell types that are responding to a ligand-receptor activation.

DE and SM clusters of each stage are ordered along the A-P axis consistent with the location of organ primordia in vivo with spatially adjacent DE and SM cell types across from one another in the diagram. To assign receptor-ligand interactions for each cell cluster, it was determined if a given cluster was responding based on having response-metagene and receptor-metagene levels ≥−1 threshold. If the responding cluster also expressed the ligand-metagene level ≥−1, an autocrine signaling was established. For paracrine signaling, adjacent cell populations within the same tissue layer and from the adjacent layer that expressed the ligand-metagene above the threshold was identified and a receptor-ligand interaction was established. The signal strength was calculated as the sum of the ligand-metagene and the response-metagene values. If this value was ≥1, the signal was considered “strong”.

Comparison of Bulk RNA-Seq vs. scRNA-Seq

Foregut tissue was dissected from E9.5 double mutant Gli2−/−;Gli3−/− (n=3) and Gli2+/−;Gli3+/− heterozygous litter mate controls (n=3). Each dissected foregut was separately used for RNA extraction, library preparation and bulk RNA-seq. These mice were of mixed strains, and the sex of the embryos were unknown. The CSBB [V3.0] (available on the World Wide Web on github.com/csbbcompbio/CSBB-v3.0) pipeline was used to align to the mouse genome [mm110] and differentially expressed transcripts between the two gene types were obtained using RUNISeq (LogFC ≥|1| and FDR ≤0.1), Differentially expressed genes were clustered using hierarchical clustering and visualized in Morpheus (available on the World Wide Web at software.broadinstitute.org/morpheus) across samples.

To compare the bulk analysis to scRNA-seq, the expression of differentially expressed genes across cells was visualized in scClusters. The ‘DoHeatmap’ function in Seurat was used. Cells were arranged according to Anterior/Posterior axis position of their respective clusters and genes were ordered as returned from the clustering order obtained above. Gene Set Enrichment Analysis (GSEA) [v3.0] was also performed to examine statistical enrichment of the differentially expressed genes in the gut tube SM (respiratory, esophagus, gastric, duodenum), pharynx, and liver SM clusters. Normalized counts of genes across cells and up/down-regulated genes from bulk RNA sequencing were used as custom gene sets to perform the GSEA analysis.

Maintenance of PSCs

Two hPSC lines were used in this study; 1) WA01-H1 human embryonic stem cells purchased from WiCell (NIH approval number NIFIFIESC-10-0043 and NTHhESC-10-0062) and 2) human iPSC72_3 generated by the CCHMC Pluripotent Stem Cell facility. Both cell lines have been authenticated as follows: i) cell identity; by STR profiling by Genetica DNA Laboratory, ii) genetic stability; by standard metaphase spread and G-banded karyotype analysis in CCHMC Cytogenetics Laboratory, and iii) functional pluripotency; cells were subjected to analysis of functional pluripotency by teratoma assay demonstrating ability to differentiate into each of the three germ layers. Both cell lines routinely tested negative for mycoplasma contamination. hPSC lines were maintained on feeder-free conditions in mTeSR1 medium (StemCell Technologies) on six-well Nunclon surface plates (Nunc) coated with Geltrex (Thermo Fisher) and maintained in mTeSR1 media (StemCell Technologies) at 37° C. with 5% CO₂. Cells were checked daily and differentiated cells were manually removed. Cells were passaged every 4 days using Dispase solution (Thermo Fisher).

Differentiation of PSCs into Mesenchyme

Differentiation of hPSCs into lateral plate mesoderm was induced using previously described methods with modifications. In brief, partially confluent hPSCs were dissociated into very fine clumps in Accutase (Invitrogen) and passaged 1:18 onto new Geltrex-coated 24-well plates for immunocytochemistry and 12-well plates for RNA preparation in mTeSR1 with 1 μM thiazovivin (Tocris) (Day 1). Next day, a brief wash with DMEM/F12 was followed with Day 0 medium (30 ng/mL Activin A (Cell Guidance Systems), 40 ng/mL BMP4 (R&D Systems), 6 CHIR99021 (Tocris), 20 ng/mL FGF2. (Thermo Fisher), 100 nM PIK90 (EMD Millipore)) for 24 hours. A basal media composed of Advanced DMEM/F12, N2, B27, 15 mM HEPES, 2 mM L-glutathione, penicillin-streptomycin was used for this Day 0 medium and all subsequent differentiations, On Day 1, a brief wash with DMEM/F12 was followed with Day 1 medium (1 μM A8301 (Tocris), 30 ng/mL BMP4, 1 μM C59 (Cellagen Technology)) for 24 hours. For cardiac mesoderm generation, cells were cultured in 1 μM A8301, 30 ng/mL BMP4, 1 μM C59, 20 ng/mL FGF2 from Day 2 to Day 4 (medium changed every day). From Day 4, cells were cultured in 200 μg/mL 2-phospho-ascorbic acid (Sigma), 1 μM XAV939 (Sigma), 30 ng/mL. BMP4 for 3 days. For splanchnic mesoderm generation, cells were cultured in 1 μM A8301, 30 ng/mL BMP4, 1 μM C59, 20 ng/mL FGF2, 2 μM RA (Sigma) from Day 2 to Day 4 (medium changed every day). To further direct regional splanChnic mesoderm, either:(1) 2 μM RA, 40 ng/mL BMP4 is used to promote STM fate for3days; (2) 2 μM RA, 2 μM purmorphamine (PMA) (Tocris) is used for 2 days, and then 2 μM RA, 2 μM PMA, 100 ng/mL Noggin (R&D Systems) is used at the last 1 day to promote esophageal/gastric mesenchyme fate; (3) 2 μM RA, 40 ng/mL BMP4, 2 μM PMA is used for 2 days, and then 2 μM RA, 40 ng/mL BMP4, 2 μM PMA, 1 μM CHIR99021 is used at the last 1 day to promote respiratory mesenchyme fate. Medium was changed every day. Similar results were obtained with WA-01 hES cells and human iPSC 72_3.

Quantitative RT-PCR

Total RNA was prepared from differentiating human ES cells by using Nucleospin kit according to manufacturer's protocol. Reverse transcription PCR was performed by Superscript VILO cDNA synthesis kit. QuantStudio 5 and 6 were used for qPCR analyses. Statistics were performed with PRISM8 (GraphPad Software). Significance was determined by one-way ANOVA, followed by Tukey's test.

Immunocytochemistry

Cells were fixed with 4% PFAJPBS for 30 min at room temperature. After perforation with 0.5% Triton X-100/PBS for 10 min, cells were incubated with 5% normal donkey serum for 2 hours. Cells were incubated with primary antibodies overnight at 4° C. Next day, cells were washed with PBS, and then incubated with secondary antibodies for 1 hour at room temperature.

Data and Code Availability

The scRNA-seg and bulk RNA-seg data (including ham, raw counts and cell annotations) are available at Gene Expression Omnibus (GEO): GSE136689 and GSE136687. All the code (scripts, R-packages, and software) and their documentation has been uploaded on the World Wide Web at github.com/ZornLab/Single-cell-transcriptomics-reveals-a-signaling-roadmap-coordinating-endoderm-and-mesoderm-lineage. All the deposited code is available to use with GPLv3.0. The scRNA-seq data can be explored on the World Wide Web at research.cchnic.org/ZornLab-singlecell.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

With respect to the use of “e.g.”, it is understood to mean “for example” and is therefore a non-limiting example.

It will be understood by those within the art that, in general, terms used herein, and especially in the appended claims (e.g., bodies of the appended claims) are generally intended as “open” terms (e.g., the term “including” is typically interpreted as “including but not limited to,” the term “having” is typically interpreted as “having at least,” the term “includes” is typically interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases is typically construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” is typically interpreted to mean “at least one” or “one or more”); the same holds true for the use of definite articles used to introduce claim recitations. In addition, even if a specific number of an introduced claim recitation is explicitly recited, those skilled in the art will recognize that such recitation is typically interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, is typically understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc, As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference for any particular disclosure referenced herein and in their entirety, and are hereby made a part of this specification. To the extent publications and patents or patent applications incorporated by reference contradict the disclosure contained in the specification, the specification is intended to supersede and/or take precedence over any such contradictory material.

### REFERENCES

Kraus, M. R. & Grapin-Botton, A. Patterning and shaping the endoderm in vivo and in culture. Curr Opin Genet Dev 22, 347-353 (2012).
Zorn, A. M. & Wells, J. M. Vertebrate endoderm development and organ formation. Annu Rev Cell Dev Biol 25, 221-251 (2009).
Le Douarin, N. & Houssaint. E. [Role of the mesoderm in the induction of the synthesis of glycogen during differentiation of the hepatic endoderm]. C R Acad Hebd Seances Acad Sci D 264, 1872-1874 (1967).
Ferretti, E. & Hadjantonakis, A. K. Mesoderm specification and diversification: from single cells to emergent tissues. Curt Opin Cell Biol 61, 110-116 (2019).
Sweetman, D., Wagstaff, L., Cooper, O., Weijer, C. & Munsterberg, A. The migration of paraxial and lateral plate mesoderm cells emerging from the late primitive streak is controlled by different Wnt signals. BMC Dev Biol 8, 63 (2008).
Barnes, R. M., Firulli, B. A., Conway, S. J., Vincentz, J. W. & Firulli, A. B. Analysis of the Hand1 cell lineage reveals novel contributions to cardiovascular, neural crest, extra-embryonic, and lateral mesoderm derivatives, Dev Dyn 239, 3086-3097 (2010).
Tang, X. et al. Transcriptorne regulation and chromatin occupancy by E2F3 and MYC in mice. Sci Data 3, 160008 (2016).
Tanaka, M. Molecular and evolutionary basis of limb field specification and limb initiation. Dev Growth Differ 55, 149-163 (2013).
Grapin-Botton, A. Antero-posterior patterning of the vertebrate digestive tract: 40 years after Nicole Le Douarin's PhD thesis. Int J Dev Biol 49, 335-347 (2005).
Francou, A. et al. Second heart field cardiac progenitor cells in the early mouse embryo. Biochim Biophys Acta 1833, 795-798 (2013).
Rana, M. S., Christoffels, V. M. & Moorman, A. F. A molecular and genetic outline of cardiac morphogenesis. Acta Physiol (Oxf) 207, 588-615 (2013).
de Soysa, T. Y. et al. Single-cell analysis of cardiogenesis reveals basis for organ-level developmental defects. Nature 572, 120-124 (2019).
Briggs, J. A. et al. The dynamics of gene expression in vertebrate embryogenesis at single-cell resolution. Science 360 (2018).
Cao, J. et al. The single-cell transcriptional landscape of mammalian organogenesis. Nature 566, 496-502 (2019).
Ibarra-Soria, X. et al. Defining murine organogenesis at single-cell resolution reveals a role for the leukotriene pathway in regulating blood progenitor formation. Nat Cell Biol 20, 127-134 (2018).
Pijuan-Sala, B. et al. A single-cell molecular map of mouse gastrulation and early organogenesis. Nature 566, 490-495 (2019),
Baron. M. et al. A Single-Cell Transcriptomic Map of the Human and Mouse Pancreas Reveals Inter- and Intra-cell Population Structure. Cell Syst 3, 346-360 e344 (2016).
Li, L. C. et al. Single-cell transcriptomic analyses reveal distinct dorsal/ventral pancreatic programs, EMBO Rep 19 (2018).
Nowotschin, S. et al. The emergent landscape of the mouse gut endoderm at single-cell resolution. Nature 569, 361-367 (2019).
Bult, C. J. et al. Mouse Genome Database (MGD) 2019. Nucleic Acids Res 47, D801-D806 (2019).
Scialdone, A. et al. Resolving early mesoderm diversification through single-cell expression profiling. Nature 535, 289-293 (2016).
Farrell, J. A. et al. Single-cell reconstruction of developmental trajectories during zebrafish embryogenesis. Science 360 (2018).
Moignard, V. et al. Decoding the regulatory network of early blood development from single-cell gene expression measurements. Nat Biotechnol 33, 269-276 (2015).
Sherwood, R. I., Chen, T. Y. & Melton, D. A. Transcriptional dynamics of endodermal organ formation. Dev Dyn 238, 29-42 (2009).
Sander, M. et al. Homeobox gene Nkx6.1 lies downstream of Nkx2.2 in the major pathway of beta-cell formation in the pancreas. Development 127, 5533-5540 (2000).
Pedersen, J. K. et al. Endodermal expression of Nkx6 genes depends differentially on Pdx1. Dev Biol 288, 487-501 (2005).
Weinreb, C., Wolock, S. & Klein, A.M. SPRING: a kinetic interface for visualizing high dimensional single-cell expression data. Bioinformatics 34, 1246-1248 (2018).
El Sebae, G. K. et al. Single-cell murine genetic fate mapping reveals bipotential hepatoblasts and novel multi-organ endoderm progenitors. Development 145 (2018).
Spence, J. R. et al. Sox17 regulates organ lineage segregation of ventral foregut progenitor cells. Dev Cell 17, 62-74 (2009).
Franklin, V. et al. Regionalisation of the endoderm progenitors and morphogenesis of the gut portals of the mouse embryo. Mech Dev 125, 587-600 (2008).
Kim, E. et al. Isl1 Regulation of Nkx2.1 in the Early Foregut Epithelium Is Required for Trachea-Esophageal Separation and Lung Lobation. Dev Cell 51, 675-683 e674 (2019).
Nasr, T. et al. Endosome-Mediated Epithelial Remodeling Downstream of Hedgehog-Gli Is Required for Tracheoesophageal Separation. Dev Cell 51, 665-674 e666 (2019).
Zaret, K. S. From Endoderm to Liver Bud: Paradigms of Cell Type Specification and Tissue Morphogenesis. Curr Top Dev Biol 117, 647-669 (2016).
Rankin, S. A. et al. A Retinoic Acid-Hedgehog Cascade Coordinates Mesoderm-Inducing Signals and Endoderm Competence during Lung Specification. Cell Reports 16, 66-78 (2016).
Rubin, L. L. & de Sauvage, F. J. Targeting the Hedgehog pathway in cancer. Nat Rev Drug Discov 5, 1026-1033 (2006).
Bort, R., Martinez-Barbera, J. P., Beddington, R. S. & Zaret, K. S. Hex homeobox gene-dependent tissue positioning is required for organogenesis of the ventral pancreas. Development 131, 797-806 (2004).
Abe, T. & Fujimori, T. Reporter mouse lines for fluorescence imaging. Dev Growth Differ 55, 390-405 (2013).
Loh. K. M. et al. Mapping the Pairwise Choices Leading from Pluripotency to Human Bone, Heart, and Other Mesoderm Cell Types. Cell 166, 451-467 (2016).
Kim, B. M., Buchner, G., Miletich, I., Sharpe, P. T. & Shivdasani, R. A. The stomach mesenchymal transcription factor Barx1 specifies gastric epithelial identity through inhibition of transient Wnt signaling. Dev Cell 8, 611-622 (2005).
Arora, R., Metzger, R. J. & Papaioannou, V. E. Multiple roles and interactions of Tbx4 and Tbx5 in development of the respiratory system. PLoS Genet 8, e1002866 (2012).
Erkan, M. et al. Organ-, inflammation- and cancer specific transcriptional fingerprints of pancreatic and hepatic stellate cells. Mol Cancer 9, 88 (2010).
Horie, M. et al. TBX4 is involved in the super-enhancer-driven transcriptional programs underlying features specific to lung fibroblasts. Am J Physiol Lung Cell Mol Physiol 314, L177-L191 (2018).
Cohen, M. et al. Lung Single-Cell Signaling Interaction Map Reveals Basophil Role in Macrophage Imprinting. Cell 175, 1031-1044 e1018 (2018).
Xie, T. et al. Single-Cell Deconvolution of Fibroblast Heterogeneity in Mouse Pulmonary Fibrosis. Cell Rep 22, 3625-3640 (2018).
Asahina, K., Zhou, B., Pu. W. T. & Tsukamoto, H. Septum transversum-derived mesothelium gives rise to hepatic stellate cells and perivascular mesenchymal cells in developing mouse liver. Hepatology 53, 983-995 (2011).
Que, J. et al. Mesothelium contributes to vascular smooth muscle and mesenchyme during lung development. Proc Natl Acad Sci USA 105, 16626-16630 (2008).
Simoes, F. C. & Riley, P. R. The ontogeny, activation and function of the epicardium during heart development and regeneration. Development 145 (2018).
Wagner, D. E. & Klein, A. M. Lineage tracing meets single-cell omics: opportunities and challenges. Nat Rev Genet (2020).
Peng, T. et al. Coordination of heart and lung co-development by a multipotent cardiopulmonary progenitor. Nature 500, 589-592 (2013).
Hoffinann, A. D., Peterson, M. A., Friedland-Little, J. M., Anderson, S. A. & Moskowitz, I. P. sonic hedgehog is required in pulmonary endoderm for atrial septation. Development 136, 1761 1770 (2009).
Adam, M., Potter. A. S. & Potter. S. S. Psychrophilic proteases dramatically reduce single-cell RNA-seq artifacts: a molecular atlas of kidney development. Development 144, 3625-3632 (2017).
Han, L. et al. Osr1 functions downstream of Hedgehog pathway to regulate foregut development. Developmental biology (2017).
Butler, A., Hoffman, P., Smibert, P., Papalexi, E. & Satija, R. Integrating single-cell transcriptomic data across different conditions, technologies, and species. Nat Biotechnol 36, 411-420 (2018).
Stuart, T. et al. Comprehensive Integration of Single-Cell Data. Cell 177, 1888-1902 e1821 (2019).
Hu, H. et al. AnimalTFDB 3.0: a comprehensive resource for annotation and prediction of animal transcription factors. Nucleic Acids Res 47, D33-D38 (2019).
Mootha, V. K. et al. PGC-lalpha-responsive genes involved in oxidative phosphorylation are coordinately downregulated in human diabetes. Nat Genet 34, 267-273 (2003).

What is claimed is:

1. A method of producing splanchnic mesoderm cells, comprising:
contacting lateral plate mesoderm cells with a TGF-beta signaling pathway inhibitor, a Win signaling pathway inhibitor, a BMP signaling pathway activator, an FGF signaling pathway activator, and a retinoic acid (RA) signaling pathway activator.

2. The method of claim 1, wherein the splanchnic mesoderm cells are human splanchnic mesoderm cells.

3. The method of claim 1-2, wherein the lateral plate mesoderm cells have been differentiated from middle primitive stream cells.

4. The method of claim 3, wherein the lateral plate mesoderm cells have been differentiated from middle primitive streak cells by contacting the middle primitive streak cells with a TGF-beta signaling pathway inhibitor, a Wnt signaling pathway inhibitor, and a BMP signaling pathway activator.

5. The method of claim 3 or 4, wherein the middle primitive streak cells have been differentiated from pluripotent stem cells.

6. The method of claim 5, wherein the middle primitive streak cells have been differentiated from pluripotent stem cells by contacting the pluripotent stem cells with a TGF-beta signaling pathway activator, a Wnt signaling pathway activator, an FGF signaling pathway activator, a BMP signaling pathway activator, and a PI3K signaling pathway inhibitor.

7. The method of any one of claims 1-6, wherein the lateral plate tnesoderm cells are contacted with A8301, BMP4, C59, FGF2, RA, or any combination thereof.

8. The method of any one of claims 1-7, wherein the lateral plate mesoderm cells are contacted for a time that is sufficient to differentiate lateral plate mesoderm cells to splanchnic mesoderm cells, and/or for a time that is or is about 36, 37, 38, 39, 40, 41, 42, 43, 44, 45. 46, 47, 48, 49, 50, 51, 52, 53, 54. 55, 56, 57, 58, 59, or 60 hours, or any time within a range defined by any two of the aforementioned times.

9. The method of any one of claims 1-8, wherein the lateral plate mesoderm cells are contacted for a time that is or is about 48 hours.

10. The method of any one of claims 1-9, wherein the splanchnic tnesoderm cells exhibit increased expression of FOXF1, HOXA1, HOXA5, or WNT2, or any combination thereof, and decreased expression of NKX2-5, ISL1, or TBX2, or any combination thereof, relative to cardiac mesoderm cells.

11. The method of any one of claims 1-10, wherein the splandmic mesoderm cells exhibit decreased expression of PAX3 or PRRX1, or both, relative to middle primitive streak cells, and/or decreased expression of CD31 relative to cardiac mesoderm cells.

12. A method of producing septum transversum cells, comprising contacting splanchnic mesoderm cells with a retinoic acid signaling pathway activator and a BMP signaling pathway activator.

13. The method of claim 12, wherein the splanchnic mesoderm cells are the splanchnic mesoderm cells of any one of claims 1-11.

14. The method of claim 12 or 13, wherein the splanchnic mesoderm cells are contacted with RA, BMP4, or both.

15. The method of any one of claims 12-1 4, wherein the splanchnic mesoderm cells are contacted for a time that is sufficient to differentiate splanchnic mesoderm cells to septum transversum cells, and/or for a time that is or is about 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times.

16. The method of any one of claims 12-1 5, wherein the splanchnic mesoderm cells are contacted for a period of time that is or is about 72 hours.

17. The method of any one of claims 12-1 6, wherein the septum transversum cells exhibit increased expression of WT1, TBX18, LHX2, UPK3B, or UPK1B, or any combination thereof, relative to cardiac mesoderm cells, splanchnic mesoderm cells, or fibroblasts, or any combination thereof.

18. The method of any one of claims 12-1 7, wherein the septum transversum cells exhibit decreased expression of MSX1, MSX2, or HAND1 or any combination thereof, relative to cardiac mesoderm cells or fibroblasts, or both.

19. The method of any one of claims 12-1 8, wherein the septum transversum cells exhibit decreased expression of HOXA1 or TBX5, or both, relative to splanchnic mesoderm cells.

20. The method of any one of claims 12-1 9, wherein the septum transversum cells exhibit decreased expression of NKX6.1 or HOXA5, or both, relative to respiratory mesenchyme cells.

21. The method of any one of claims 12-20, Wherein the septum transversum cells exhibit decreased expression of NKX3.2, MSC, BARX1, WNT4, or HOXA5, or any combination thereof, relative to esophageal/gastric mesenchyme cells.

22. The method of any one of claims 12-21, wherein the septum transversum cells account for about 60%, 65%, 70%, 75%, 80%, 85%, or 90% of the total cells differentiated from the splanchnic mesoderm cells.

23. A method of producing fibroblasts, comprising contacting splanchnic mesoderm cells with a retinoic acid signaling pathway activator, a BMP signaling pathway activator, and a Wnt signaling pathway activator.

24. The method of claim 23, wherein the splanchnic mesoderm cells are the splanchnic mesoderm cells of any one of claims 1-11.

25. The method of claim 23 or 24, wherein the splanchnic mesoderm cells are contacted with RA, BMP4, CHIR99021, or any combination thereof.

26. The method of any one of claims 23-25, wherein the fibroblasts are liver fibroblasts.

27. The method of any one of claims 23-26, wherein the splanchnic mesoderm cells are contacted for a time that is sufficient to differentiate splanchnic mesoderm cells to fibroblasts, and/or for a time that is or is about 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times.

28. The method of any one of claims 23-27, wherein the splanchnic mesoderm cells are contacted for a period of time that is or is about 72 hours.

29. The method of any one of claims 23-28, wherein the fibroblasts exhibit increased expression of MSX1, MSX2, or HAND1 or any combination thereof, relative to splanchnic mesoderm cells or septum transversum cells, or both.

30. The method of any one of claims 23-29, wherein the fibroblasts exhibit decreased expression of WT1, TBX18, LHX2, or UPK1B, or any combination thereof, relative to septum transversum cells.

31. The method of any one of claims 23-30, wherein the fibroblasts exhibit decreased expression of NKX6.1, HOXA5, or LHX2, or any combination thereof, relative to respiratory mesenchyme cells.

32. The method of any one of claims 23-31, wherein the fibroblasts exhibit decreased expression of NKX3.2, MSC, BARX1, WNT4, or HOXA5, or any combination thereof, relative to esophageal/gastric mesenchyme cells.

33. A method of producing respiratory mesenchyme cells, comprising a) contacting splanchnic mesoderm cells with a retinoic acid signaling pathway activator, a BMP signaling pathway activator, a hedgehog (HH) signaling pathway activator, and a Wnt signaling pathway activator.

34. The method of claim 33, wherein the splanchnic mesoderm cells are contacted for a time that is sufficient to differentiate splanchnic mesoderm cells to respiratory mesenchyme cells, and/or for a time that is or is about 60, 61, 62, 63, 64, 65, 66, 67, 68, 69, 70, 71, 72, 73, 74, 75, 76, 77, 78, 79, 80, 81, 82, 83, or 84 hours, or any period of time within a range defined by any two of the aforementioned times.

35. The method of claim 33 or 34, wherein the splanchnic mesoderm cells are contacted for a period of time that is or is about 72 hours.

36. The method of claim 33, wherein step a) is a second step, and further comprising a first step of contacting the splanchnic mesoderm cells with a retinoic acid signaling pathway activator, a BMP signaling pathway activator, and a HH signaling pathway activator prior to the second step.

37. The method of claim 36, wherein the splanchnic mesoderm cells are contacted for a time that is sufficient to differentiate splanchnic mesoderm cells to respiratory mesenchyme cells, and/or for a time or is about 36, 37, 38, 39, 40, 41, 42, 43, 44, 45. 46, 47, 48, 49, 50, 51, 52, 53, 54, 55, 56, 57, 58, 59, or 60 hours, or any period of time within a range defined by any two of the aforementioned times for the first step.

38. The method of claim 36 or 37, wherein the splanchnic mesoderm cells are contacted for a period of time that is or is about 48 hours for the first step.

39. The method of any one of claims 36-38, wherein the splanchnic mesoderm cells are contacted for a time that is sufficient to differentiate splanchnic mesoderm cells to respiratory mesenchyme cells, and/or for a time or is about 12, 13, 14, 15, 16, 17, 18, 19, 20, 21, 22, 23, 24, 25, 26, 27, 28, 29, 30, 31, 32, 33, 34, 35, or 36 hours, or any period of time within a range defined by any two of the aforementioned times for the second step.

40. The method of any one of claims 36-39, wherein the splanchnic mesoderm cells are contacted for a period of time that is or is about 24 hours for the second step.

41. The method of any one of claims 33-40, wherein the splanchnic mesoderm cells are the splanchnic mesoderm cells of any one of claims 1-11.

42. The method of any one of claims 33-41, wherein the splanchnic mesoderm cells are contacted with RA, BMP4, PMA, CHIR99021, or any combination thereof.

43. The method of any one of claims 33-42, wherein the respiratory mesenchyme cells exhibit increased expression of NKX6-1, TBX5, HOXA1 HOXA5, FOXF1, LHX2, or WNT2, or any combination thereof, relative to cardiac endoderm cells, splanchnic mesoderm cells, or esophageal/gastric mesenchyme cells, or any combination thereof.

44. The method of any one of claims 33-43, wherein the respiratory mesenchyme cells exhibit decreased expression of WNT2, WT1, TBX18, LHX2, or UPK1B, or any combination thereof, relative to septum transversum cells.

45. The method of any one of claims 33-44, wherein the respiratory mesenchyme cells exhibit decreased expression of WNT2, MSX1, or MSX2, or any combination thereof, relative to fibroblast cells.

46. A method of producing esophageal/gastric mesenchyme cells, comprising a) contacting splanchnic mesoderm cells with a retinoic acid signaling pathway activator, a BMP signaling pathway inhibitor, and a HH signaling pathway activator.

47. The method of claim 46, wherein the splanchnic mesoderm cells are contacted for a time that is sufficient to differentiate splanchnic mesoderm cells to esophageal/gastric mesenchyme cells, and/or for a time or is about 60, 61, 62, 63, 64, 65, 66, 67, 68, 69, 70, 71, 72, 73, 74. 75, 76, 77, 78, 79, 80, 81, 82, 83, or 84 hours, or any period of time within a range defined by any two of the aforementioned times.

48. The method of claim 46 or 47, wherein the splanchnic mesoderm cells are contacted for a period of time that is or is about 72 hours.

49. The method of claim 46, wherein step a) is a second step, and further comprising a first step of contacting the splanchnic mesoderm cells with a retinoic acid signaling pathway activator and a HH signaling pathway activator prior to the second step.

50. The method of claim 49, wherein the splanchnic mesoderm cells are contacted for a time that is sufficient to differentiate splanchnic mesoderm cells to esophageal/gastric mesenchyme cells, and/or for a time or is about 36, 37, 38. 39, 40, 41, 42, 43, 44, 45. 46, 47, 48, 49, 50, 51, 52, 53, 54, 55, 56, 57, 58, 59, or 60 hours, or any period of time within a range defined by any two of the aforementioned times for the first step.

51. The method of claim 49 or 50, wherein the splanchnic mesoderm cells are contacted for a period of time that is or is about 48 hours for the first step.

52. The method of any one of claims 49-51, wherein the splanchnic mesoderm cells are contacted for a time that is sufficient to differentiate splanchnic mesoderm cells to esophageal/gastric mesenchyme cells, and/or for a time or is about 12, 13, 14, 15, 16, 17, 18, 19, 20, 21, 22, 23, 24, 25, 26, 27, 28, 29, 30, 31, 32, 33, 34, 35, or 36 hours, or any period of time within a range defined by any two of the aforementioned times for the second step.

53. The method of any one of claims 49-52, wherein the splanchnic mesoderm cells are contacted for a period of time that is or is about 24 hours for the second step.

54. The method of claim 46-53, wherein the splanchnic mesoderm cells are the splanchnic mesoderm cells of any one of claims 1-11.

55. The method of any one of claims 46-54, wherein the splanchnic mesoderm cells are contacted with RA, Noggin, PMA, or any combination thereof.

56. The method of any one of claims 46-55, wherein the esophageal/gastric mesenchyme cells exhibit increased expression of MSC, BARX1, WNT4, HOXA1, FOXF1, or NKX3-2, or any combination thereof, relative to cardiac endoderm cells, splanchnic mesoderm cells, or respiratory mesenchyme cells, or any combination thereof.

57. The method of any one of claims 46-56, wherein the esophageal/gastric mesenchyme cells exhibit decreased expression of WNT2, TBX5, MSX1, MSX2, or LHX2, or any combination thereof, relative to septum transversum cells, fibroblasts, or respiratory mesenchyme cells, or any combination thereof.

58. The method of any one of claims 1-57, wherein the TGE-beta signaling pathway inhibitor is selected from the group consisting of A8301, RepSox, LY365947, and SB431542.

59. The method of any one of claims 1-58, wherein the TGE-beta signaling pathway inhibitor is A8301.

60. The method of any one of claims 1-59, wherein the TGF-beta signaling pathway inhibitor is contacted at a concentration of 0.1, 0.2. 0.3, 0.4, 0.5, 0.6, 0.7, 0.8, 0.9, 1, 1.1, 1.2, 1.3, 1.4, 1.5, 1.6, 1.7. 1.8, 1.9, or 2 μM, or about 0.1, 0.2, 0.3, 0.4, 0.5, 0.6, 0.7. 0.8, 0.9, 1, 1.1, 1.2, 1.3, 1.4, 1.5, 1.6, 1.7, 1.8, 1.9, or 2 μM, or any concentration within a range defined by any two of the aforementioned concentrations.

61. The method of any one of claims 1-60, wherein the TGF-beta signaling pathway inhibitor is contacted at concentration of 1 μM or about 1 μM.

62. The method of any one of claims 1-61, wherein the Wnt signaling pathway inhibitor is selected from the group consisting of C59, PNU 74654, KY-02111, PRI-724, FH-535, DIF-1, and XAV939.

63. The method of any one of claims 1-62, wherein the Wnt signaling pathway inhibitor is C59.

64. The method of any one of claims 1-63, wherein the Wnt signaling pathway inhibitor is contacted at a concentration of 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations.

65. The method of any one of claims 1-64, wherein the Wnt signaling pathway inhibitor is contacted at a concentration of 1 μM or about 1 μM.

66. The method of any one of claims 1-65, wherein the BMP signaling pathway activator is selected from the group consisting of BMP1, BMP2, BMP3, BMP4, BMP5, BMP6, BMP7, BMP8a, BMP8b, BMP10, BMP11, BMP15, IDE1 and IDE2.

67. The method of any one of claims 1-66, wherein the BMP signaling pathway activator is BMP4.

68. The method of any one of claims 1-67, wherein the BMP signaling pathway activator is contacted at a concentration of 15, 16, 17, 18, 19, 20, 21, 22, 23, 24, 25, 26, 27, 28, 29, 30, 31, 33, 34, 35, 36, 37, 38, 39, 40, 41, 42, 43, 44, or 45 ng/mL or about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 ng/mL, or any concentration within a range defined by any two of the aforementioned concentrations.

69. The method of any one of claims 1-68, wherein the BMP signaling pathway activator is contacted at a concentration of 30 ng/mL or about 30 ng/mL.

70. The method of any one of claims 1-69, wherein the FGF signaling pathway activator is selected from the group consisting of FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, and FGF23.

71. The method of any one of claims 1-70, wherein the FGF signaling pathway activator is FGF2.

72. The method of any one of claims 1-71, wherein the FGF signaling pathway activator is contacted at a concentration of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 ng/mL, or about 5, 6, 7, 8, 9, 10, 11, 12, 13, 14, 15, 16, 17, 18, 19, 20, 21, 22, 23, 24, 25, 26, 27, 28, 29, 30, 31. 32, 33, 34, or 35 ng/mL, or any concentration within a range defined by any two of the aforementioned concentrations.

73. The method of any one of claims 1-72, wherein the FGF signaling pathway activator is contacted at a concentration of 20 ng/mL, or about 20 ng/mL.

74. The method of any one of claims 1-73, wherein the RA signaling pathway activator is selected from the group consisting of retinoic acid, all-trans retinoic acid, 9-cis retinoic acid, CD437, EC23, BS 493, TTNPB, and AM580.

75. The method of any one of claims 1-74, wherein the RA signaling pathway activator is RA.

76. The method of any one of claims 1-75, wherein the RA signaling pathway activator is contacted at a concentration of 1, 1.1, 1.2, 1.3, 1.4, 1.5, 1.6, 1.7, 1.8, 1.9, 2, 2.1, 2.2, 2.3, 2.4, 2.5, 2.6, 2.7, 2.9, or 3 μM or about 1, 1.1, 1.2, 1.3, 1.4, 1.5, 1.6, 1.7, 1.8, 1.9, 2.1, 2.2, 2.3, 2.4, 2.5, 2.6, 2.7, 2.9, or 3 μM, or any concentration within a range defined by any two of the aforementioned concentrations.

77. The method of any one of claims 1-76, wherein the RA signaling pathway activator is contacted at a concentration of 2 μM or about 2 μM.

78. The method of any one of claims 1-77, wherein the Wnt signaling pathway activator is selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, and TWS119.

79. The method of any one of claims 1-78, wherein the Wnt signaling pathway activator is CHIR99021.

80. The method of any one of claims 1-79, wherein the Wnt signaling pathway activator is contacted at a concentration of 0.01, 0.1, 1, 3, 4, 5, 6, 7, 8, 9, 10, 11, 12, 13, 14, 15, 16, 17, 18, 19, or 20 μM, or about 0.01, 0.1, 1 3, 4, 5, 6, 7, 8, 9, 10, 11, 12, 13, 14, 15, 16, 17, 18, 19, or 20 μM, or any concentration within a range defined by any two of the aforementioned concentrations.

81. The method of any one of claims 1-80, wherein the Wnt signaling pathway activator is contacted at a concentration of 1, 2, 3, 4, 5, 6, 7, 8, 9, or 10 μM, or about 1, 2, 3, 4, 5, 6, 7, 8, 9, or 10 μM, or any concentration within a range defined by any two of the aforementioned concentrations.

82. The method of any one of claims 1-81, wherein the HH signaling pathway activator is selected from the group consisting of SHH, IHH, DHH, PMA, GSA 10, and SAG.

83. The method of any one of claims 1-82, wherein the HH signaling pathway activator is PMA.

84. The method of any one of claims 1-83, wherein the HH signaling pathway activator is contacted at a concentration of 1, 1.1, 1.2, 1.3, 1.4, 1.5, 1.6, 1.7, 1.8, 1.9, 2, 2.1, 2,2, 2.3, 2.4, 2.5, 2.6, 2,7, 2.8, 2.9, or 3 μM or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM, or any concentration within a range defined by any two of the aforementioned concentrations.

85. The method of any one of claims 1-84, wherein the signaling pathway activator is contacted at a concentration of 2 μM or about 2 μM.

86. The method of any one of claims 1-85, wherein the BMP signaling pathway inhibitor is selected from the group consisting of Noggin, RepSox, LY364947, LDN193189, and SB431542.

87. The method of any one of claims 1-86, wherein the BMP signaling pathway inhibitor is Noggin.

88. The method of any one of claims 1-87, wherein the BMP signaling pathway inhibitor is contacted at a concentration of 50, 60, 70, 80, 90, 100, 110, 120, 130, 140, or 150 ng/mL or about 50, 60, 70, 80, 90, 100, 110, 120, 130, 140, or 150 ng/mL, or any concentration within a range defined by any two of the aforementioned concentrations.

89. The method of any one of claims 1-88, wherein the BMP signaling pathway inhibitor is contacted at a concentration of 100 ng/mL or about 100 ng/mL.

90. The splanchnic mesoderm cells produced by the method of any one of claims 1-11.

91. The septum transversum cells produced by the method of any one of claims 12-22.

92. The fibroblasts produced by the method of any one of claims 23-32.

93. The respiratory mesenchyme cells produced by the method of any one of claims 33-45.

94. The esophageal/gastric mesenchyme cells produced by the method of any one of claims 46-57.
